(19) United States
(12) Patent Application Publication
(10) Pub. No.: US 2023/0063705 A1
(43) Pub. Date: Mar. 2, 2023

(54) METHODS AND KITS FOR AMPLIFICATION AND DETECTION OF NUCLEIC ACIDS

(71) Applicant: Qingdao Navid Biotechnology Co., LTD., Qingdao (CN)

(72) Inventors: Chao SHI, Qingdao (CN); Cuiping MA, Qingdao (CN); Mengmeng LIU, Qingdao (CN); Yang LI, Qingdao (CN)

(21) Appl. No.: 17/794,467

(22) PCT Filed: Jan. 20, 2021
(86) PCT No.: PCT/CN2021/072900

§ 371 Date: Jul. 21, 2022

Related U.S. Application Data
(60) Provisional application No. 62/971,871, filed on Feb. 7, 2020.
(60) Provisional application No. 63/013,455, filed on Apr. 21, 2020.

(30) Foreign Application Priority Data
Jan. 21, 2020 (CN) 202010071234.0
Apr. 17, 2020 (CN) 202010307560.7

(51) Int. Cl. C12Q 1/686; C12Q 1/6806

(52) U.S. Cl. CPC C12Q 1/686; C12Q 1/6806; C12Q 2600/178

(57) ABSTRACT

Provided herein is a method for denaturation bubble-mediated target nucleic acid amplification and related kits and uses thereof. The method facilitates the generation of denaturation bubbles in a duplex target nucleic acid molecule through the application of swift temperature changes during a thermal cycle, thereby accelerating the strand exchange amplification (SEA) reaction. The kits comprise specially designed primers and polymerase configured for performing the method. The methods and kits disclosed herein can be used under various scenarios, such as diagnosis of infectious or genetic diseases, sample quality control, and single nucleotide polymorphism (SNP) profiling.

### CROSS-REFERENCE TO RELATED APPLICATION

This application claims the benefit of Chinese patent application No: 202010071234.0 filed Jan. 21, 2020, Chinese patent application No.: 202010307560.7 filed Apr. 17, 2020, U.S. provisional patent application No. 62/971,871 filed Feb. 7, 2020, and U.S. provisional patent application No. 63/013,455 filed Apr. 21, 2020, the contents of each of which are herein incorporated by reference in their entirety.

### FIELD

The field of invention relates to the field of biotechnology, in particular to a modified denaturation bubble-mediated target nucleic acid amplification method and related kits and uses.

### 1. BACKGROUND

Nucleic acids including Deoxyribonucleic acid (DNA) and ribonucleic acid (RNA) are the basic elements of all life forms. DNA carrying genetic information and is responsible for encoding proteins constitutes of amino acids as the basic units. RNA plays important roles in genetic coding, decoding, regulation and expression. Hence, nucleic acids are widely used as important biomarkers during biological research and medical diagnosis. In this context, nucleic acid amplification technology provides an important foundation for the detection of pathogenic microorganisms, determination of types and sources of biological materials (such as meat), and other genetic examinations. The establishment of a simple, easy-to-operate, sensitive and fast method of nucleic acid amplification and detection has been the main goal in the field of biological examination.

Since the discovery of the polymerase chain reaction (PCR), research effort has long been focused on modifying and improving the technology to improve the speed and sensitivity of the technology. However, due to limitations of polymerase kinetics and the heating and cooling rates of thermal cyclers, conventional PCR reactions typically take an hour or more to complete. Improving these two factors (enzyme system and instrument) has been the key for shortening amplification time. With the improvement of enzyme systems and commercialization of thermal cyclers capable of rapid temperature shifts, the PCR amplification time has been reduced from 4 hours in the early times to about 1 hour currently. There is no commercial instrument available to further reduce the reaction time.

Presently, commercially available thermal cyclers typically perform energy transfer through a typical 25-50 μL reaction system, which volume limits the energy transfer rate, making it difficult to further reduce the reaction time. This has led some researchers to use infrared lamps, infrared lasers in droplets, microwaves, microwave field in droplets, or other forms of energy that are significantly absorbed by liquid samples to achieve rapid heating. However, these non-contact heating methods lack sensitivity and accuracy, and are not suitable for use in research laboratories. On the other hand, researchers have used microfluidic technology to reduce the volume of the reaction chamber, thus increasing the reaction speed by reducing the time scale for transferring energy to and from the sample. For example, new platforms for small-volume PCR, such as droplet PCR, etc., can achieve rapid PCR amplification, but are not without difficulties such as small throughput and process limitations. Despite various attempts, methods that are based on rapid PCR amplification (such as Rapid-Cycle PCR and Integrated Extreme Real-Time PCR) are still limited by the development and commercialization of thermal cycling devices. There has been no report on whether the above problems can be solved by optimizing the current isothermal amplification technology.

In this context, isothermal amplification technology (such as Loop Mediated Isothermal Amplification (LAMP), Helicase-dependent Isothermal Deoxyribonucleic Acid (HDA), Strand Exchange Amplification (SEA), etc.) has been developed as an alternative to PCR. LAMP technology is well known for its high sensitivity and specificity, but the reaction is easily contaminated, and the design of primers is difficult to detect targets of high mutant rates. The HDA technology requires two enzymes in the reaction system. The dual enzyme system is prone to non-specific amplification that confounds interpretation of results. These shortcomings have limited the popularization of these technologies to some extent.

Denaturation bubble mediated strand exchange amplification (SEA) refers to the isothermal amplification mediated by denaturation bubbles that are formed spontaneously in duplex DNA (a phenomenon known as DNA respiration). Only a pair of upstream and downstream primers are needed for exponential amplification. The primers can invade the denatured portions (“bubbles”) of a partially unwound DNA molecule, extending and replacing the original complementary strand under the action of a polymerase to produce the amplicon. CN 109136337 A describes isothermal SEA capable of amplifying and detecting 1.0×10⁻¹⁴M nucleic acids in a sample.

Despite various prior efforts, there still exists a need for the establishment of a high-throughput and stable nucleic acid amplification technology that is suitable for traditional laboratory uses. The present disclosure meets this need.

### 2. SUMMARY

Denaturation bubble mediated strand exchange amplification (SEA) is an isothermal nucleic acid amplification method based on the spontaneous formation of denatured regions (“bubbles”) in double-stranded DNA (dsDNA) due to ambient thermal fluctuations. A pair of oligonucleotide primers then invade a denaturation bubble, binding to unwound single-stranded DNA in the bubble, extending and replacing the original complementary strand under the action of a polymerase to produce the amplicon. Hence, the method, which utilizes small denaturation bubbles spontaneously formed without heating up the sample, is thought to advantageously eliminate the need for a thermal cycler, and performs the PCR reaction under one temperature typically selected for optimal polymerase activity (Shi et al. “Triggered isothermal PCR by denaturation bubble-mediated strand exchange amplification” Chem Commun (Camb) (2016) 4; 52(77):11551-4).

The present disclosure is based, at least partially, on the surprising discovery that modifying the isothermal SEA method by swiftly changing the reaction temperature, even within a small range of a few degrees, significantly increases the efficiency and speed of amplification by thousands of folds. The present method is hence referred to as “accelerated SEA” in certain passages of this application. Accordingly, in one aspect of the present disclosure, provided herein are methods for the amplification and detection of a target nucleic acid in a sample. In some embodiments, the method comprises contacting a polymerase and a pair of oligonucleotide primers with the sample, thereby forming an amplification mixture; wherein the primers are configured to specifically hybridize to the target nucleic acid molecule; subjecting the amplification mixture to a number of thermal cycles between a first temperature and a second temperature, thereby amplifying a sequence of the target nucleic acid molecule through polymerase chain reaction (PCR); and wherein the difference between the first and second temperatures is less than about 30° C. In some embodiments, the method comprises contacting a polymerase and a pair of oligonucleotide primers with the sample, thereby forming an amplification mixture; wherein the primers are configured to specifically hybridize to the target nucleic acid molecule; subjecting the amplification mixture to a number of thermal cycles between a first temperature and a second temperature, thereby amplifying a sequence of the target nucleic acid molecule through polymerase chain reaction (PCR); and wherein the difference between the first and second temperatures is less than about 25° C. In some embodiments, the method comprises contacting a polymerase and a pair of oligonucleotide primers with the sample, thereby forming an amplification mixture; wherein the primers are configured to specifically hybridize to the target nucleic acid molecule; subjecting the amplification mixture to a number of thermal cycles between a first temperature and a second temperature, thereby amplifying a sequence of the target nucleic acid molecule through polymerase chain reaction (PCR); and wherein the difference between the first and second temperatures is less than about 20° C. In some embodiments, the present method further comprises detecting the amplified sequence. In some embodiments, the present method further comprises making a diagnosis based on the detection.

In specific embodiments, the difference between the first and second temperature is about 10-15° C. In more specific embodiments, the difference between the first and second temperatures is about 10° C., about 11° C., about 12° C., about 13° C., about 14° C., or about 15° C.

In some embodiments, the polymerase has an optimal temperature for catalyzing primer extension during the PCR. In specific embodiments, the optimal temperature is in the range of ±6° C. of the first temperature. In specific embodiments, the optimal temperature is in the range of ±6° C. of the second temperature. In specific embodiments, the optimal temperature is between the first and second temperatures.

In some embodiments, the sequence of the target nucleic acid molecule to be amplified by the present method has a first melting temperature, and wherein the first temperature is in the range of ±5° C. of the first melting temperature. In some embodiments, the pair of oligonucleotide primers have an average melting temperature, and wherein the second temperature is in the range of ±5° C. of the average melting temperature. In some embodiments, the average melting temperature is within ±5° C. of the optimal temperature of the polymerase. In some embodiments, one of the pair of oligonucleotide primers has a second melting temperature and the other one of the pair of oligonucleotide primers has a third melting temperature, and wherein difference between the second and third melting temperatures is less than about 3° C.

In some embodiments, the first melting temperature is determined using a computer algorithm based on the sequence of the target nucleic acid molecule. Additionally or alternatively, in some embodiments, the second melting temperature is determined using a computer algorithm based on the sequence of the oligonucleotide primer. Additionally or alternatively, in some embodiments, the third melting temperature is determined using a computer algorithm based on the sequence of the oligonucleotide primer. In some embodiments, the computer algorithm is selected from NUPACK, DNAMelt, NOVOPRO, BLAST, Primer Premier, AlignMiner, Oligo, PerlPrimer, Primer3Web and DNAstar. In some embodiments, the present method further comprises determining the first, second, and/or third melting temperature.

In some embodiments, the polymerase is a thermostable polymerase. In some embodiments, the polymerase has strand displacement activity. In some embodiments, the polymerase has reverse transcriptase activity.

In some embodiments, the polymerase is Bst DNA polymerase, or an isomerase thereof, or a functional derivative having at least 80% sequence identity thereof. In specific embodiments, the polymerase is Bst DNA polymerase Large Fragment, or isomerase thereof, or a functional mutant having at least 80% sequence identity thereof. In specific embodiments, the polymerase is full length Bst DNA Polymerase, Bst DNA Polymerase Large Fragment, Bst 2.0 DNA polymerase, Bst 2.0 WarmStart DNA Polymerase, or Bst 3.0 DNA polymerase. In specific embodiments, where the polymerase is any of the polymerases described in this paragraph, the first temperature is in the range of about 68-78° C., and the second temperature is in the range of about 55-69° C.

In some embodiments, the polymerase is DNA polymerase I, or an isomerase thereof, or a functional mutant having at least 80% sequence identity thereof. In some embodiments, the polymerase is DNA Polymerase I Large (Klenow) Fragment, or an isomerase thereof, or a functional mutant having at least 80% sequence identity thereof. In specific embodiments, the polymerase is wild-type DNA Polymerase I, DNA Polymerase I Large (Klenow) Fragment, or Klenow exo⁻. In specific embodiments, where the polymerase is any of the polymerases described in this paragraph, the first temperature is in the range of about 50-60° C., and the second temperature is in the range of about 30-40° C.

In some embodiments, the polymerase is a Vent DNA polymerase, or an isomerase thereof, or a functional mutant having at least 80% sequence identity thereof. In specific embodiments, the polymerase is Vent DNA polymerase, Vent (exo⁻) DNA polymerase, Deep Vent DNA polymerase, or Deep Vent (exo⁻) DNA polymerase. In specific embodiments, where the polymerase is any of the polymerases described in this paragraph, the first temperature is in the range of about 70-80° C., and the second temperature is in the range of about 55-70° C.

In some embodiments, the polymerase is a phi29 DNA polymerase, or an isomerase thereof, or a functional mutant having at least 80% sequence identity thereof. In specific embodiments, where the polymerase is any of the polymerases described in this paragraph, the first temperature is selected from the range of about 40-55° C., and the second temperature is selected from the range of about 20-37° C.

In some embodiments, the polymerase is a Taq DNA polymerase, or an isomerase thereof, or a functional mutant having at least 80% sequence identity thereof. In specific embodiments, the polymerase is Taq DNA polymerase, Hot Start Taq DNA Polymerase, EpiMark Hot Start Taq DNA Polymerase, OneTaq DNA Polymerase, OneTaq Hot Start DNA Polymerase, LongAmp Taq DNA Polymerase, or LongTaq DNA Polymerase. In specific embodiments, where the polymerase is any of the polymerases described in this paragraph, the first temperature is in the range of about 70-88° C., and the second temperature is in the range of about 58-70° C.

In some embodiments, the ratio of the length of the amplified sequence and the length of at least one of the primers is in the range of about 30-60%. In specific embodiments, the amplified sequence is about 20-50 base pair (bp) long. In specific embodiments, the primer is about 10 to about 25 nucleotides (nt) long.

In some embodiments, at least one of the primers has a melting temperature within ±5° C. of the optimal temperature of the polymerase. In some embodiments, the difference between the melting temperatures of the primers are less than 1° C. In some embodiments, at least one of the primers has a G/C content in the range of about 40% to about 60%. In some embodiments, the difference between the G/C content of the primers are less than 20%. In some embodiments, at least one of the primers has an elongation terminus where the polymerase adds nucleotides during the PCR, and wherein the primer has G or C at the elongation terminus. In some embodiments, at least one of the primers has an elongation terminus where the polymerase adds nucleotides during the PCR, and wherein the primer has a G/C content of at least 40% in a continuous 5-nucletoide region including the elongation terminus.

In some embodiments, each thermal cycle comprises incubating the amplification mixture at the first temperature for less than 2 s and incubating the amplification mixture at the second temperature for less than 2 s. In some embodiments, each thermal cycle further comprises a total ramp time of less than 10 s. In some embodiments, each thermal cycle comprises incubating the amplification mixture at the first temperature for about 1 s and incubating the amplification mixture at the second temperature for about 1 s, and wherein the ramp time is less than 2 s. In some embodiments, the method completes at least 35 thermal cycles in less than 10 minutes, or completes at least 40 thermal cycles in less than 8 minutes.

In some embodiments, the amplification mixture further comprises dUTPs. In some embodiments, the amplification mixture does not contain dTTPs. In some embodiments, the amplification mixture further comprises uracil-DNA glycosylase (UDG). In some embodiments, the amplification mixture further comprises a single strand binding protein (SSB). In some embodiments, the amplification mixture further comprises polyethylene glycol.

In some embodiments, the amplification mixture comprises the target nucleic acid of no more than 1.0×10⁻¹² M. In some embodiments, the amplification mixture comprises less than 10 copies of the target nucleic acid. In some embodiments, the amplification mixture comprises the polymerase at a concentration of no less than 0.1 U/μL. In some embodiments, the amplification mixture comprises at least one of the primers at a concentration of no less than 1.0×10⁻⁶ M. In some embodiments, the amplification mixture comprises polyethylene glycol of at least 0.5% by volume. In some embodiments, the amplification mixture comprises the SSB at a concentration of at least 1 μg/mL. In some embodiments, the amplification mixture has a volume of about 1-30 μL.

In some embodiments, the subjecting step is performed by loading the amplification mixture onto a microfluidic device capable of cooling and heating the amplification mixture at a speed of at least 10° C./s. In some embodiments, the target nucleic acid is a double-stranded nucleic acid molecule, or single-stranded nucleic acid molecule. In some embodiments, the target nucleic acid is DNA or RNA.

In another aspect of the present disclosure, provided herein are also related methods for detecting a target nucleic acid molecule in a sample. In some embodiments, the method comprises contacting a polymerase and a pair of oligonucleotide primers with the sample, thereby forming an amplification mixture; wherein the primers are configured to specifically hybridize to the target nucleic acid molecule; subjecting the amplification mixture to a number of thermal cycles between a first temperature and a second temperature, thereby amplifying a sequence of the target nucleic acid molecule through polymerase chain reaction (PCR); wherein the difference between the first and second temperatures is less than about 30° C.; and detecting the amplified sequence in the amplification mixture. In some embodiments, the method comprises contacting a polymerase and a pair of oligonucleotide primers with the sample, thereby forming an amplification mixture; wherein the primers are configured to specifically hybridize to the target nucleic acid molecule; subjecting the amplification mixture to a number of thermal cycles between a first temperature and a second temperature, thereby amplifying a sequence of the target nucleic acid molecule through polymerase chain reaction (PCR); wherein the difference between the first and second temperatures is less than about 25° C.; and detecting the amplified sequence in the amplification mixture. In some embodiments, the method comprises contacting a polymerase and a pair of oligonucleotide primers with the sample, thereby forming an amplification mixture; wherein the primers are configured to specifically hybridize to the target nucleic acid molecule; subjecting the amplification mixture to a number of thermal cycles between a first temperature and a second temperature, thereby amplifying a sequence of the target nucleic acid molecule through polymerase chain reaction (PCR); wherein the difference between the first and second temperatures is less than about 20° C.; and detecting the amplified sequence in the amplification mixture. In specific embodiments, the detecting is performed every 1, 2, 5 or 10 thermal cycles. In specific embodiments, the detecting is performed by detecting a fluorescent signal reflective of the amount of the amplified sequence in the amplification mixture.

In another aspect of the present disclosure, provided herein are also related methods for diagnosing an infection by a pathogen in a subject. In some embodiments, the method comprises providing a nucleic acid containing sample collected from the subject; contacting a polymerase and a pair of oligonucleotide primers with the sample, thereby forming an amplification mixture; wherein the primers are configured to amplify a pathogenic sequence indicative of the pathogen infection; subjecting the amplification mixture to a number of thermal cycles between a first temperature and a second temperature, thereby amplifying the pathogenic sequence through polymerase chain reaction (PCR); wherein the difference between the first and second temperatures is less than about 30° C.; and detecting the presence or absence of the amplified sequence in the amplification mixture. In some embodiments, the method comprises providing a nucleic acid containing sample collected from the subject; contacting a polymerase and a pair of oligonucleotide primers with the sample, thereby forming an amplification mixture; wherein the primers are configured to amplify a pathogenic sequence indicative of the pathogen infection; subjecting the amplification mixture to a number of thermal cycles between a first temperature and a second temperature, thereby amplifying the pathogenic sequence through polymerase chain reaction (PCR); wherein the difference between the first and second temperatures is less than about 25° C.; and detecting the presence or absence of the amplified sequence in the amplification mixture. In some embodiments, the method comprises providing a nucleic acid containing sample collected from the subject; contacting a polymerase and a pair of oligonucleotide primers with the sample, thereby forming an amplification mixture; wherein the primers are configured to amplify a pathogenic sequence indicative of the pathogen infection; subjecting the amplification mixture to a number of thermal cycles between a first temperature and a second temperature, thereby amplifying the pathogenic sequence through polymerase chain reaction (PCR); wherein the difference between the first and second temperatures is less than about 20° C.; and detecting the presence or absence of the amplified sequence in the amplification mixture. In specific embodiments, the sample contains extracted genomic nucleic acid of the subject, or cell-free nucleic acid from the subject. In specific embodiments, the sample is a bodily fluid sample. In specific embodiments, the pathogen is virus, bacteria, fungi or parasite.

In another aspect of the present disclosure, provided herein are also related methods for detecting a genetic alteration in a subject. In some embodiments, the method comprises providing a nucleic acid containing sample collected from the subject; contacting a polymerase and a pair of oligonucleotide primers with the sample, thereby forming an amplification mixture; wherein the primers are configured to amplify a target sequence from the subject's genome suspected of containing the genetic alteration; subjecting the amplification mixture to a number of thermal cycles between a first temperature and a second temperature, thereby amplifying the target sequence through polymerase chain reaction (PCR); wherein the difference between the first and second temperatures is less than about 30° C.; and sequencing the amplified sequence to determine the presence of absence of the genetic alteration. In specific embodiments, the genetic alteration is a gene mutation selected from nucleotide substitute, deletion, insertion or copy number variation. In some embodiments, the method comprises providing a nucleic acid containing sample collected from the subject; contacting a polymerase and a pair of oligonucleotide primers with the sample, thereby forming an amplification mixture; wherein the primers are configured to amplify a target sequence from the subject's genome suspected of containing the genetic alteration; subjecting the amplification mixture to a number of thermal cycles between a first temperature and a second temperature, thereby amplifying the target sequence through polymerase chain reaction (PCR); wherein the difference between the first and second temperatures is less than about 25° C.; and sequencing the amplified sequence to determine the presence of absence of the genetic alteration. In specific embodiments, the genetic alteration is a gene mutation selected from nucleotide substitute, deletion, insertion or copy number variation. In some embodiments, the method comprises providing a nucleic acid containing sample collected from the subject; contacting a polymerase and a pair of oligonucleotide primers with the sample, thereby forming an amplification mixture; wherein the primers are configured to amplify a target sequence from the subject's genome suspected of containing the genetic alteration; subjecting the amplification mixture to a number of thermal cycles between a first temperature and a second temperature, thereby amplifying the target sequence through polymerase chain reaction (PCR); wherein the difference between the first and second temperatures is less than about 20° C.; and sequencing the amplified sequence to determine the presence of absence of the genetic alteration. In specific embodiments, the genetic alteration is a gene mutation selected from nucleotide substitute, deletion, insertion or copy number variation. In specific embodiments, the genetic alteration is single nucleotide polymorphism. In some embodiments, the method further comprises diagnosing or prognosing a genetic condition associated with the genetic alteration.

In another aspect of the present disclosure, provided herein are also kits for performing the present methods. In some embodiments, a kit for amplifying a target nucleic acid molecule is provided. In some embodiments, the kit comprises a plurality of components comprising a thermostable polymerase and a pair or oligonucleotide primers, wherein the pair of primers are configured to amplify, through polymerase chain reaction (PCR), an amplification region of about 20-50 base pairs (bp) in the target nucleic acid; and wherein the thermostable polymerase comprises strand displacement activity.

In some embodiments of the kits, at least one of the primers have a melting temperature within ±5° C. of the optimal temperature of the thermostable polymerase. In some embodiments, at least one of the primers has a G/C content in the range of about 40%-60%. In some embodiments, the difference between the G/C content of the primers are less than 20%. In some embodiments, each primer comprises an elongation terminus where the polymerase adds nucleotides during the PCR, and wherein at least one of the primers has a G/C content of at least 40% in a continuous 5-nucleotide region including the elongation terminus. In some embodiments, each primer comprises an elongation terminus where the polymerase adds nucleotides during the PCR, and wherein at least one of the primers has G or C at the elongation terminus. In some embodiments, at least one of the primers is about 10-25 nucleotides long.

In some embodiments of the kits, the polymerase is Bst DNA polymerase, or an isomerase thereof, or a functional mutant having at least 80% sequence identity thereof. In some embodiments, the polymerase is Bst DNA polymerase Large Fragment, or an isomerase thereof, or a functional mutant having at least 80% sequence identity thereof. In some embodiments, the polymerase is full length Bst DNA Polymerase, Bst DNA Polymerase Large Fragment, Bst 2.0 DNA polymerase, Bst 2.0 WarmStart DNA Polymerase, or Bst 3.0 DNA polymerase.

In some embodiments, the polymerase is DNA Polymerase I, or an isomerase thereof, or a functional mutant having at least 80% sequence identity thereof. In some embodiments, the polymerase is DNA Polymerase I Large (Klenow) Fragment, or an isomerase thereof, or a functional mutant having at least 80% sequence identity thereof. In some embodiments, the polymerase is wild-type DNA Polymerase I, DNA Polymerase I Large (Klenow) Fragment, or Klenow exo⁻.

In some embodiments, the polymerase is a Vent DNA polymerase, or an isomerase thereof, or a functional mutant having at least 80% sequence identity thereof. In some embodiments, the polymerase is Vent DNA polymerase, Vent (exo⁻) DNA polymerase, Deep Vent DNA polymerase, or Deep Vent (exo⁻) DNA polymerase. In some embodiments, the polymerase is a phi29 DNA polymerase, or an isomerase thereof, or a functional mutant having at least 80% sequence identity thereof.

In some embodiments, the polymerase is a Taq DNA polymerase, or an isomerase thereof, or a functional mutant having at least 80% sequence identity thereof. In some embodiments, the polymerase is Taq DNA polymerase, Hot Start Taq DNA Polymerase, EpiMark Hot Start Taq DNA Polymerase, OneTaq DNA Polymerase, OneTaq Hot Start DNA Polymerase, LongAmp Taq DNA Polymerase, or LongTaq DNA Polymerase.

In some embodiments, the kit further comprises dUTPs. In some embodiments, the kit does not contain dTTPs. In some embodiments, the kit further comprises uracil-DNA glycosylase (UDG). In some embodiments, the kit further comprises a buffer solution suitable for the polymerase. In some embodiments, the further comprises polyethylene glycol. In some embodiments, the kit further comprises a single strand binding protein (SSB), preferably a thermal stable SSB. In some embodiments, the SSB protein is originated from bacteria or phage. In some embodiments, the SSB protein is selected from T4 phage 32 SSB, T7 phage 2.5 SSB, phi phage 29 SSB, E. coli SSB, or functional derivative thereof.

In some embodiments, the plurality of components of the kit are (a) contained in one container, and the kit further comprises an instruction of adding a suitable amount of sample to form an amplification mixture; or (b) contained in at least two separate containers, and wherein the kit further comprises an instruction of mixing the components in the separate containers and a suitable amount of sample to form an amplification mixture. In some embodiments, the amplification mixture comprises the polymerase at a concentration of no less than 0.1 U/μL. In some embodiments, the amplification mixture comprises at least one of the primers at a concentration of no less than 1.0×10⁻⁶M. In some embodiments, the amplification mixture comprises polyethylene glycol of about 0.5%-10% by volume. In some embodiments, the amplification mixture comprises the SSB at a concentration of about 1-50 μg/mL. In some embodiments, the amplification mixture has a volume of about 1-30 μL.

In some embodiments, the kit further comprises an instruction for performing the PCR using a thermal cycling protocol comprising a number of thermal cycles, wherein each thermal cycle comprises incubation at a first temperature for no more than 2 s, and incubation at a second temperature for no more than 2 s, and wherein the difference between the first and second temperatures is less than 30° C. In some embodiments, the kit further comprises an instruction for performing the PCR using a thermal cycling protocol comprising a number of thermal cycles, wherein each thermal cycle comprises incubation at a first temperature for no more than 2 s, and incubation at a second temperature for no more than 2 s, and wherein the difference between the first and second temperatures is less than 25° C. In some embodiments, the kit further comprises an instruction for performing the PCR using a thermal cycling protocol comprising a number of thermal cycles, wherein each thermal cycle comprises incubation at a first temperature for no more than 2 s, and incubation at a second temperature for no more than 2 s, and wherein the difference between the first and second temperatures is less than 20° C. In specific embodiments, the polymerase is full length Bst DNA Polymerase, Bst DNA Polymerase Large Fragment, Bst 2.0 DNA polymerase, Bst 2.0 WarmStart DNA Polymerase, or Bst 3.0 DNA polymerase, and wherein the first temperature is in the range of about 68-78° C., and the second temperature is in the range of about 55-69° C. In specific embodiments, the polymerase is full length Bst DNA Polymerase, Bst DNA Polymerase Large Fragment, Bst 2.0 DNA polymerase, Bst 2.0 WarmStart DNA Polymerase, or Bst 3.0 DNA polymerase, and where each thermal cycle comprises incubation at the first temperature selected from the range of about 72-76° C. for about 1 s, and incubation at the second temperature selected from the range of about 61-65° C. for about 1 s, and the total ramp time of less than 2 s, and wherein the total reaction time is less than 8 minutes.

In specific embodiments, the polymerase is wild-type DNA Polymerase I, DNA Polymerase I Large (Klenow) Fragment, or Klenow exo⁻, and wherein the first temperature is in the range of about 50-60° C., and the second temperature is in the range of about 30-40° C. In specific embodiments, the polymerase is Vent DNA polymerase, Vent (exo) DNA polymerase, Deep Vent DNA polymerase, or Deep Vent (exo⁻) DNA polymerase, and wherein the first temperature is in the range of about 70-80° C., and the second temperature is in the range of about 55-70° C. In specific embodiments, the polymerase is phi29 DNA polymerase, and wherein the first temperature is selected from the range of about 40-55° C., and the second temperature is selected from the range of about 20-37° C.

In specific embodiments, the polymerase is Taq DNA polymerase, Hot Start Taq DNA Polymerase, EpiMark Hot Start Taq DNA Polymerase, OneTaq DNA Polymerase, OneTaq Hot Start DNA Polymerase, LongAmp Taq DNA Polymerase or LongTaq DNA Polymerase, and wherein the first temperature is selected from the range of about 70-88° C. and the second temperature is selected from the range of about 58-70° C.

In some embodiments, each thermal cycle further comprises a total ramp time of less than 10 s. In some embodiments, the number of thermal cycles is less than 40 cycles and the thermal cycling protocol further comprises a total reaction time of less than 10 minutes.

In some embodiments, the amplification region has a first melting temperature, and wherein the first temperature is in the range of ±5° C. of the first melting temperature. In some embodiments, the pair of primers in the kit have an average melting temperature, and wherein the second temperature is in the range of ±5° C. of the average melting temperature. In some embodiments, one of the pair of oligonucleotide primers has a second melting temperature and the other one of the pair of oligonucleotide primers has a third melting temperature, and wherein difference between the second and third melting temperatures is less than about 3° C.

### 3. BRIEF DESCRIPTION OF THE FIGURES

FIG. 1 is a schematic illustration of the mechanism of denaturation bubble mediated strand exchange amplification of duplex nucleic acid molecules, such as DNA.

FIG. 2 shows real-time amplification curves of a target sequence in the hypervariable region of Listeria monocytogenes 16s rRNA encoding gene under swift thermal cycles between 76° C. and 62° C. The X-axis shows the amplification time in minutes (min), indicative of the number of thermal cycles that the amplification reaction has gone through, and Y-axis shows the fluorescent signal intensity in relative fluorescence units (RFU), indicative of the amount of amplicons produced by the reaction. Different symbols represent different primer concentrations as indicated.

FIG. 3 shows real-time amplification curves of a target sequence in the hypervariable region of Listeria monocytogenes 16s rRNA encoding gene under swift thermal cycles between 76° C. and 62° C. The X-axis shows the amplification time in minutes (min), indicative of the number of thermal cycles that the amplification reaction has gone through, and Y-axis shows the fluorescent signal intensity in relative fluorescence units (RFU), indicative of the amount of amplicons produced by the reaction. Different symbols represent different polymerase concentrations as indicated.

FIG. 4 shows real-time amplification curves of a target sequence in the hypervariable region of Listeria monocytogenes 16s rRNA encoding gene under the different thermal cycles between a high temperature in the range of 74 to 78° C. and a low temperature of 62° C. The X-axis shows the number of thermal cycles that the amplification reaction has gone through, and Y-axis shows the fluorescent signal intensity in relative fluorescence units (RFU), indicative of the amount of amplicons produced by the reaction. Different symbols represent different temperatures as indicated.

FIG. 5 shows real-time amplification curves of a synthetic DNA fragment under swift thermal cycles between 76° C. and 62° C. The X-axis shows the amplification time in minutes (min), indicative of the number of thermal cycles that the amplification reaction has gone through, and Y-axis shows the fluorescent signal intensity in relative fluorescence units (RFU), indicative of the amount of amplicons produced by the reaction. Different symbols represent the different target concentrations as indicated.

FIG. 6A shows real-time amplification curves of a synthetic RNA fragment under swift thermal cycles between 76° C. and 62° C. The X-axis shows the amplification time in minutes (min), indicative of the number of thermal cycles that the amplification reaction has gone through, and Y-axis shows the fluorescent signal intensity in relative fluorescence units (RFU), indicative of the amount of amplicons produced by the reaction. Different symbols represent the target concentrations as indicated.

FIG. 6B is a polyacrylamide gel electrophoresis (PAGE) image showing an amplicon produced by the accelerated SEA reactions described in Example 2. Lane M shows a series of DNA molecular-weight size markers (DNA ladder), and the bands corresponding to 20 bp and 40 bp DNA fragments are indicated on the figure. The remaining lanes show presence of a specific 43 bp amplicon produced by three repeated reactions having the initial target concentration of 1.0×10⁻¹² M, and the lack of the specific amplicon in the negative control, as indicated. The bands of less than 20 bp reflects remaining primer molecules.

FIG. 7A shows real-time amplification curves of a target sequence in the hypervariable region of Listeria monocytogenes 16s rRNA encoding gene under swift thermal cycles between 76° C. and 62° C. The X-axis shows the amplification time in minutes (min), indicative of the number of thermal cycles that the amplification reaction has gone through, and Y-axis shows the fluorescent signal intensity in relative fluorescence units (RFU), indicative of the amount of amplicons produced by the reaction. Different symbols represent different initial target concentrations as indicated.

FIG. 7B is a polyacrylamide gel electrophoresis (PAGE) image showing a 43-bp amplicon produced by the accelerated SEA reactions described in Example 3. Lane M shows a series of DNA molecular-weight size markers (DNA ladder), and the bands corresponding to 20 bp and 40 bp DNA fragments are indicated on the figure. The remaining lanes show presence of a 43 bp specific amplicon produced by reactions having different initial target concentrations, and the lack of the specific amplicon in the negative control, as indicated.

FIG. 7C real-time amplification curves of a target sequence in the hypervariable region of Listeria monocytogenes 16s rRNA encoding gene under the constant reaction temperature of 62° C. The X-axis shows the amplification time in minutes (min), indicative of the number of thermal cycles that the amplification reaction has gone through, and Y-axis shows the fluorescent signal intensity in relative fluorescence units (RFU), indicative of the amount of amplicons produced by the reaction. Different symbols represent different initial target concentrations as indicated.

FIG. 8 shows real-time amplification curves of a 50 bp fragment in the Staphylococcus aureus 16s RNA encoding gene under swift thermal cycles between 76° C. and 61° C. The X-axis shows the amplification time in minutes (min), indicative of the number of thermal cycles that the amplification reaction has gone through, and Y-axis shows the fluorescent signal intensity in relative fluorescence units (RFU), indicative of the amount of amplicons produced by the reaction. Different symbols represent the target concentrations as indicated.

FIG. 9 illustrates a commercial manufacture's description and recommendation of several Bst DNA polymerases, which may be used in connection with the present methods and kits in certain embodiments.

FIG. 10A-E show real-time amplification curves of SEA reactions using a purified M. pneumonia 16S rRNA fragment as template and five different primer pairs (Mp1-Mp5) at a series of constant reaction temperatures (57° C., 59° C., 61° C., 63° C., and 65° C.). Particularly, FIG. 10A shows amplification curves using primer pair Mp1 (Tₘ of about 65° C.) at the five different reaction temperatures as indicated. Particularly, FIG. 10B shows amplification curves using primer pair Mpg (Tₘ of about 63° C.) at the five different reaction temperatures as indicated. Particularly, FIG. 10C shows amplification curves using primer pair Mp3 (Tₘ of about 61° C.) at the five different reaction temperatures as indicated. Particularly, FIG. 10D shows amplification curves using primer pair Mp4 (Tₘ of about 59° C.) at the five different reaction temperatures as indicated. Particularly, FIG. 10E shows amplification curves using primer pair Mp1 (Tₘ of about 57° C.) at the five different reaction temperatures as indicated. The X-axis shows the amplification time in minutes (min), and Y-axis shows the fluorescent signal intensity, indicative of the amount of amplicons produced by the reaction. Negative control (NTC) are also shown.

FIG. 10F shows real-time amplification curves of SEA reactions using extracted M. pneumonia genomic materials as template and primer pair Mp3 at the five different reaction temperatures as indicated. The X-axis shows the amplification time in minutes (min), and Y-axis shows the fluorescent signal intensity, indicative of the amount of amplicons produced by the reaction. Negative control (NTC) are also shown.

FIG. 11A shows real-time amplification curves of SEA reactions using different pair of primers (Ct1-Ct3) that are specific to a target sequence in the C. trachoma 16S rRNA. The X-axis shows the amplification time in minutes (min), and Y-axis shows the fluorescent signal intensity, indicative of the amount of amplicons produced by the reaction. Negative control (NTC) are also shown.

FIG. 11B shows real-time amplification curves of SEA reactions using different primer pairs (Sd1-Sd3) that are specific to a target sequence in the S. domestica 18S rRNA. The X-axis shows the amplification time in minutes (min), and Y-axis shows the fluorescent signal intensity, indicative of the amount of amplicons produced by the reaction. Negative control (NTC) are also shown.

FIG. 12A shows real-time amplification curves of SEA reactions using different primer pairs (Mp3, Mp6, Mp7) that are specific to a target sequence in the M. pneumonia's 16S rRNA. The X-axis shows the amplification time in minutes (min), and Y-axis shows the fluorescent signal intensity, indicative of the amount of amplicons produced by the reaction. Negative control (NTC) are also shown.

FIG. 12B shows real-time amplification curves of SEA reactions using different primer pairs (Ct1, Ct4 and Ct5) that are specific to a target sequence in the C. trachoma's 16S rRNA. The X-axis shows the amplification time in minutes (min), and Y-axis shows the fluorescent signal intensity, indicative of the amount of amplicons produced by the reaction. Negative control (NTC) are also shown.

FIG. 13A shows real-time amplification curves of SEA reactions using different primer pairs (Ct1, Ct6 and Ct2) that are specific to a target sequence in the C. trachoma 16S rRNA. The X-axis shows the amplification time in minutes (min), and Y-axis shows the fluorescent signal intensity, indicative of the amount of amplicons produced by the reaction. Negative control (NTC) are also shown.

FIG. 13B shows real-time amplification curves of SEA reactions using different primer pairs (Bc1-Bc3) that are specific to a target sequence in the B. cereus 16S rRNA. The X-axis shows the amplification time in minutes (min), and Y-axis shows the fluorescent signal intensity, indicative of the amount of amplicons produced by the reaction. Negative control (NTC) are also shown.

FIG. 14 shows real time amplification curves of SEA reactions using different primer pairs (Sal and Sat) that are specific to a target sequence is the S. aureus 16S rRNA. The X-axis shows the amplification time in minutes (min), and Y-axis shows the fluorescent signal intensity, indicative of the amount of amplicons produced by the reaction. Negative control (NTC) are also shown.

FIG. 15 shows real time amplification curves of accelerated SEA reactions using a microfluidic device. The X-axis shows the number of thermal cycles that the amplification reaction has gone through, and Y-axis shows the fluorescent signal intensity in relative fluorescence units (RFU), indicative of the amount of amplicons produced by the reaction. Different symbols represent different target molecule concentrations as indicated.

FIG. 16 shows real time amplification curves of accelerated SEA reactions using dUTPs or dTTPs. The X-axis shows the amplification time in minutes (min), and Y-axis shows the fluorescent signal intensity in relative fluorescence units (RFU), indicative of the amount of amplicons produced by the reaction. Negative control group (NTC) is also shown.

FIG. 17 is a gel image showing UDG enzyme digesting of uracil-containing amplification product.

FIG. 18 shows real time amplification curves of accelerated SEA reactions using dUTPs with or without the UDG enzyme. The X-axis shows the amplification time in minutes (min), and Y-axis shows the fluorescent signal intensity in relative fluorescence units (RFU), indicative of the amount of amplicons produced by the reaction.

FIG. 19 shows real time amplification curves of a synthesized target DNA fragment under swift thermal cycles between 76° C. and 61° C. The X-axis shows the amplification time in minutes (min), and Y-axis shows the fluorescent signal intensity in relative fluorescence units (RFU), indicative of the amount of amplicons produced by the reaction.

FIG. 20 shows real time amplification curves of a target sequence in the human β-actin gene under swift thermal cycles between 76° C. and 61° C. The X-axis shows the amplification time in minutes (min), and Y-axis shows the fluorescent signal intensity in relative fluorescence units (RFU), indicative of the amount of amplicons produced by the reaction.

FIG. 21 shows real time amplification curves of a synthesized target DNA fragment under swift thermal cycles between 76° C. and 55° C. The X-axis shows the amplification time in minutes (min), and Y-axis shows the fluorescent signal intensity in relative fluorescence units (RFU), indicative of the amount of amplicons produced by the reaction. Different symbols represent the target concentrations as indicated.

FIG. 22 shows real time amplification curves of a synthesized target microRNA fragment under swift thermal cycles between 60° C. and 34° C. The X-axis shows the amplification time in minutes (min), and Y-axis shows the fluorescent signal intensity in relative fluorescence units (RFU), indicative of the amount of amplicons produced by the reaction. Different symbols represent the target concentrations as indicated.

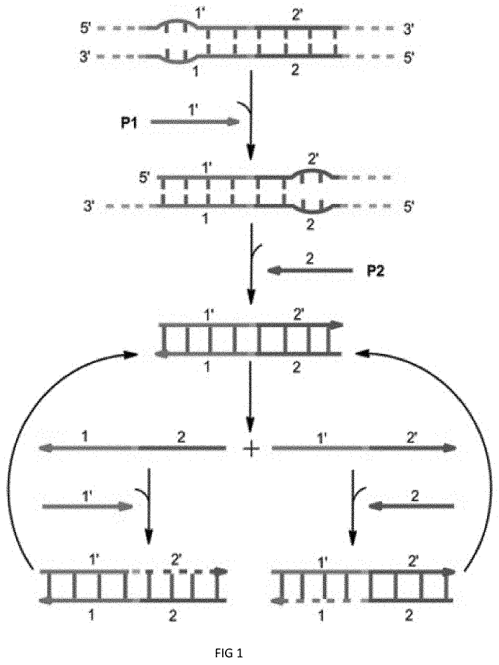

### 4. DETAILED DESCRIPTION

Provided herein are methods for amplifying and detecting a target nucleic acid in an sample.

In one aspect of the present disclosure, provided herein are methods for amplifying a target nucleic acid. The method comprises contacting a thermostable polymerase and a pair of oligonucleotide primers with the sample, thereby forming an amplification mixture; subjecting the amplification mixture to a number of thermal cycles between a first temperature in the range of about 68-78° C. and a second temperature in the range of about 55-69° C., thereby amplifying a sequence of the target nucleic acid through polymerase chain reaction (PCR).

In another aspect of the present disclosure, provided herein are kits for performing the present method. The kit comprises at least a thermostable polymerase and a pair of oligonucleotide primers with the sample, and optionally instruction for using the kit to perform the present methods. Additional features of the present disclosure will become apparent to those skilled in the art upon consideration of the following detailed description of particular embodiments.

### 4.1 General Techniques

Techniques and procedures described or referenced herein include those that are generally well understood and/or commonly employed using conventional methodology by those skilled in the art, such as, for example, the widely utilized methodologies described in Sambrook et al., Molecular Cloning: A Laboratory Manual (3d ed. 2001); Current Protocols in Molecular Biology (Ausubel et al. eds., 2003).

### 4.2 Terminology

Unless described otherwise, all technical and scientific terms used herein have the same meaning as is commonly understood by one of ordinary skill in the art. For purposes of interpreting this specification, the following description of terms will apply and whenever appropriate, terms used in the singular will also include the plural and vice versa. All patents, applications, published applications, and other publications are incorporated by reference in their entirety. In the event that any description of terms set forth conflicts with any document incorporated herein by reference, the description of term set forth below shall control.

The singular terms “a,” “an,” and “the” as used herein include the plural reference unless the context clearly indicates otherwise.

The term “about” as used herein means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 5%. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

The term “amino acid” refers to naturally occurring and non-naturally occurring alpha-amino acids, as well as alpha-amino acid analogs and amino acid mimetics that function in a manner similar to the naturally occurring alpha-amino acids. Naturally encoded amino acids are the 22 common amino acids (alanine, arginine, asparagine, aspartic acid, cysteine, glutamine, glutamic acid. glycine, histidine, isoleucine, leucine, lysine, methionine, phenylalanine, proline, serine, threonine, tryptophan, tyrosine, valine, pyrrolysine and selenocysteine). Amino acid analogs or derivatives refers to compounds that have the same basic chemical structure as a naturally occurring amino acid, i.e., a carbon that is bound to a hydrogen, a carboxyl group, an amino group, and a side chain R group, such as, homoserine, norleucine, methionine sulfoxide, methionine methyl sulfonium. Such analogs have modified R groups (such as, norleucine) or modified peptide backbones, but retain the same basic chemical structure as a naturally occurring amino acid. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The term “conservative substitution” as used herein refers to replacement of one amino acid residue by another, biologically similar residue. Examples of conservative substitutions include the substitution of one hydrophobic residue such as lie, Val, Leu or Met for another, or the substitution of one polar residue for another such as between Arg and Lys, between Glu and Asp or between Gin and Asn, and the like. In some instances, the replacement of an ionic residue by an similarly or oppositely charged ionic residue such as Asp by Lys has been termed conservative in the art in that those ionic groups are thought to merely provide solubility assistance. The terms “nonionic” and “ionic” residues are used herein in their usual sense to mean those amino acid residues that normally either bear no charge or normally bear a charge, respectively, at physiological pH values. Exemplary nonionic residues include Thr and Gin, while exemplary ionic residues include Arg and Asp.

The terms “non-natural amino acid” or “non-proteinogenic amino acid” or “unnatural amino acid” refer to alpha-amino acids that contain different side chains (different R groups) relative to those that appear in the twenty-two common or naturally occurring amino acids listed above. In addition, these terms also can refer to amino acids that are described as having D-stereochemistry, rather than L-stereochemistry of natural amino acids, despite the fact that some amino acids do occur in the D-stereochemical form in Nature (e.g., D-alanine and D-serine).

The term “Bst DNA polymerase” as used herein refers to the wild-type DNA polymerase originated from Bacillus stearothermophilus, or a mutated or truncated version thereof that retains at least the polymerase and strand displacement activities. The enzyme can be isolated from B. stearothermophilus or synthetically produced. One exemplary embodiment of Bst DNA polymerase that is particularly useful for the present disclosure is Bst DNA Polymerase, Large Fragment, which has been reported to have good strand displacement activity at around 65° C., and have an intrinsic reverse transcriptase activity. (Shi et al. “Innate reverse transcriptase activity of DNA polymerase for isothermal RNA direct detection.” J. Am. Chem. Soc. (2015) 137, 13804-13806), and devoid of 5′→3′ exonuclease activity. Other useful embodiments of Bst DNA polymerase according to the present disclosure include but are not limited to Full Length Bst DNA Polymerase, Large Fragment Bst DNA Polymerase and mutated Bst DNA polymerases such as Bst 2.0 DNA polymerase, Bst 2.0 WarmStart DNA Polymerase, and Bst 3.0 DNA polymerase commercialized by New England BioLabs®.

The term “functional derivative” of a reference enzyme or protein used herein refers to an enzyme or protein that has a different amino acid sequence as compared to the reference enzyme or protein, but retain the same functionality of the reference enzyme or protein. In some instance, the term is used with respect to one or more activities of interest, and as long as a variant retains the activities of interest of the reference, the variant can be considered a functional derivative, even though the variant may be devoid of other function or activity of the reference. In some instance, a functional derivative can retain the same activity of a reference, even though the extent of level of activity of the derivative is enhanced or reduced, and such a derivative can still be considered as a functional derivative of the reference.

The term “GIC contents” in a term of art in molecular biology and genetics, and refers to the percentage of nitrogenous bases in a DNA or RNA molecule that are either guanine (G) or cytosine (C).

The term “genetic polymorphism” refers to the phenomenon where two or more DNA sequences coexist in the same interbreeding population.

The term “identity” refers to a relationship between the sequences of two or more polypeptide molecules or two or more nucleic acid molecules, as determined by aligning and comparing the sequences. Percent (%) “sequence identity” with respect to a reference polynucleotide sequence is defined as the percentage of nucleotides in a candidate sequence that are identical with the nucleotides in the reference polynucleotide sequence, after aligning the sequences and introducing gaps, if necessary, to achieve the maximum percent sequence identity, and not considering any substitutions as part of the sequence identity. Alignment for purposes of determining percent nucleic acid sequence identity can be achieved in various ways that are within the skill in the art, for instance, using publicly available computer software such as BLAST, BLAST-2, ALIGN, or MEGALIGN (DNAStar, Inc.) software. Those skilled in the art can determine appropriate parameters for aligning sequences, including any algorithms needed to achieve maximal alignment over the full length of the sequences being compared. Exemplary parameters for determining relatedness of two or more sequences using the BLAST algorithm, for example, can be as set forth below. Briefly, sequence alignments can be performed using BLASTP version 2.0.8 (Jan. 5, 1999) and the following parameters: Matrix: 0 BLOSUM62; gap open: 11; gap extension: 1; x_dropoff: 50; expect: 10.0; wordsize: 3; filter: on. Nucleic acid sequence alignments can be performed using BLASTN version 2.0.6 (Sep. 16, 1998) and the following parameters: Match: 1; mismatch: −2; gap open: 5; gap extension: 2; x_dropoff: 50; expect: 10.0; wordsize: 11; filter: off. Those skilled in the art will know what modifications can be made to the above parameters to either increase or decrease the stringency of the comparison, for example, and determine the relatedness of two or more sequences.

The terms “oligonucleotide” and “nucleic acid” refer to oligomers of deoxyribonucleotides (e.g., DNA) or ribonucleotides (e.g., RNA) and polymers thereof in either single- or double-stranded form. Unless specifically limited, the term encompasses nucleic acids containing known analogues of natural nucleotides which have similar binding properties as the reference nucleic acid and are metabolized in a manner similar to naturally occurring nucleotides. Unless specifically limited otherwise, the term also refers to oligonucleotide analogs including PNA (peptidonucleic acid), analogs of DNA used in antisense technology (phosphorothioates, phosphoroamidates, and the like). Unless otherwise indicated, a particular nucleic acid sequence also implicitly encompasses conservatively modified variants thereof (including but not limited to, degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer, M. A., et al., Nucleic Acid Res., 1991, 19, 5081-1585; Ohtsuka, E. et al., J. Biol. Chem., 1985, 260, 2605-2608; and Rossolini, G. M., et al., Mol. Cell. Probes, 1994, 8, 91-98). As used herein, nucleic acid as used herein can refer to, without limitation, DNA, RNA, cDNA, gDNA, rRNA, ssDNA, dsDNA, DNA-RNA hybrid, etc.

The term “or” as used herein means any one member of a particular list and also includes any combination of members of that list, unless specified otherwise or the context where the term appears indicates or suggests otherwise.

The term “phi29 DNA polymerase” as used herein refers to the wild-type replicative polymerase originated from Bacillus subtilis phage phi29 (D29), or a mutated or truncated version thereof that retains at least the polymerase and strand displacement activities. The enzyme can be isolated from phage phi29 or synthetically produced.

The term “polymerase chain reaction” or PCR as used herein refers to a chain reaction catalyzed by a nucleic acid polymerase, where the nucleic acid strands produced in earlier rounds of the reaction is used as templates for subsequent rounds of the reaction.

The term “probe,” “primer,” or “oligonucleotide” as used herein refers to a single-stranded DNA or RNA molecule of defined sequence that can base-pair to a second DNA or RNA molecule that contains a complementary sequence (the “target”). Hybridization is the association of two single strands of complementary nucleic acid to form a hydrogen bonded double strand. The stability of the resulting hybrid depends upon the length, G/C content, nearest neighbor stacking energy, and the extent of the base-pairing that occurs. The extent of base-pairing is affected by parameters such as the degree of complementarity between the probe and target molecules and the degree of stringency of the hybridization conditions. The degree of hybridization stringency is affected by parameters such as temperature, salt concentration, and the concentration of organic molecules such as formamide, and is determined by methods known to one skilled in the art. Probes, primers, and oligonucleotides may be detectably labeled, either radioactively, fluorescently, or non-radioactively, by methods well-known to those skilled in the art. dsDNA binding dyes (dyes that fluoresce more strongly when bound to double-stranded DNA than when bound to single-stranded DNA or free in solution) may be used to detect dsDNA. It is understood that a “primer” is specifically configured to be extended by a polymerase, whereas a “probe” or “oligonucleotide” may or may not be so configured.

The terms “polypeptide” and “protein” are used interchangeably herein to refer to a polymer of greater than about fifty (50) amino acid residues. That is, a description directed to a polypeptide applies equally to a description of a protein, and vice versa. The terms apply to naturally occurring amino acid polymers as well as amino acid polymers in which one or more amino acid residues is a non-naturally occurring amino acid, e.g., an amino acid analog. As used herein, the terms encompass amino acid chains of any length greater than 50 amino acid residues, including full length proteins (e.g., full length polymerase), wherein the amino acid residues are linked by covalent peptide bonds.

The term “peptide” as used herein refers to a polymer chain containing between two and fifty (2-50) amino acid residues. The terms apply to naturally occurring amino acid polymers as well as amino acid polymers in which one or more amino acid residues is a non-naturally occurring amino acid, e.g., an amino acid analog or non-natural amino acid.

The term “sample” as used herein, refers to an animal; a tissue or organ from an animal; a cell (either within a subject, taken directly from a subject, or a cell maintained in culture or from a cultured cell line); a cell lysate (or lysate fraction) or cell extract; a solution containing one or more molecules derived from a cell, cellular material, or viral material (e.g. a polypeptide or nucleic acid); or a solution containing a naturally or non-naturally occurring nucleic acid, which is assayed as described herein. A sample may also be any body fluid or excretion (for example, but not limited to, blood, urine, stool, saliva, tears, bile) that contains cells, cell components, or nucleic acids.

The term “specifically hybridizes” or its grammatical variant as used herein means that a primer recognizes and physically interacts (that is, base-pairs) with a substantially complementary nucleic acid (for example, a sample nucleic acid) under high stringency conditions, and does not substantially base pair with other nucleic acids. The phrase “high stringency conditions” refers to conditions that allow hybridization comparable with that resulting from the use of a DNA probe of at least 40 nucleotides in length, in a buffer containing 0.5 M sodium phosphate, pH 7.2, 7% SDS, 1 mM EDTA, and 1% BSA (Fraction V), at a temperature of 65° C., or a buffer containing 48% formamide, 4.8×SSC, 0.2 M Tris-Cl, pH 7.6, IX Denhardt's solution, 10% dextran sulfate, and 0.1% SDS, at a temperature of 42° C. Other conditions for high stringency hybridization, such as for PCR, northern, Southern, or in situ hybridization, DNA sequencing, etc., are well known by those skilled in the art of molecular biology. (Ausubel et al., “Current Protocols in Molecular Biology,” John Wiley & Sons, New York, N.Y., 1998).

The term “strand displacement” or its grammatical variant as used herein is a term of art, and refers to the ability of a polymerase to displace a downstream complementary nucleic acid strand encountered during its synthesis of a new complementary strand. The outcome is the production of a duplex nucleic acid molecule containing the original template strand and the newly synthesized complementary strand, while the original complementary strand is removed. Several DNA polymerases have been reported to have varying degrees of strand displacement activity. For example, phi29 DNA polymerase has a very strong ability to for strand displace. Other examples of strand displacing polymerase include DNA Polymerase I, Large (Klenow) Fragment, Vent® DNA Polymerase, and Bacillus stearothermophilus (Bst) DNA Polymerase, Large Fragment.

Some strand displacement polymerases are also known to be thermally stable. For example, Bst DNA Polymerase, Large Fragment exhibits good strand displacement activity at elevated temperatures, such as around 65° C. Additional such examples include but are not limited to DNA Polymerase I, Large (Klenow) Fragment exhibiting good strand displacement activity at elevated temperatures, such as around 37° C., Vent® DNA Polymerase exhibiting good strand displacement activity at elevated temperatures, such as around 75° C.

Several strand displacement polymerases are commercially available. For example, New England BioLabs® has commercialized several engineered versions of Bst DNA Polymerases. The manufacture's description and recommendation for these products (obtained from www.neb.com/faqs/0001/01/01/when-should-bst-dna-polymerase-be-the-enzyme-of-choice) is reproduced in FIG. 9 solely for the illustration purpose. Zeng et al. describes a mutant version of DNA Polymerase I, Large (Klenow) Fragment (Klenow exo⁻) that lacks the 5′→3′ exonuclease activity but retains the strand displacement activity (Zeng et al. “Strand Displacement Amplification for Multiplex Detection of Nucleic Acids” (2018); DOI: 10.5772/intechopen.80687). While these enzymes can be used in connection with the present methods and kits, the present disclosure is by no means limited to these exemplary enzymes of commercial products. As would be understood by those skilled in the art, other enzymes currently known in the art or to be discovered in the future that satisfy the description of desirable activities as disclosed herein are also contemplated by and included in the present disclosure.

The terms “subject” and “patient” may be used interchangeably. As used herein, in certain embodiments, a subject is a mammal, such as a non-primate (e.g., cow, pig, horse, cat, dog, rat, etc.) or a primate (e.g., monkey and human). In specific embodiments, the subject is a human.

The term “thermostable polymerase” as used herein refers to a polymerase that is stable and active in the temperature range of about 50-80° C., and is capable of catalyzing elongation of a primer annealed to a template strand by adding nucleotides complementary to the template sequence to produce a new strand. The synthesis can be initiated at the 3′ end of a primer and proceed towards the 5′ end of the template strand (5′→3′ polymerase activity), until synthesis terminates, producing nucleic acid molecules of different lengths. Alternatively, the synthesis can be initiated at the 5′ end of a primer and proceed towards the 3′ end of the template strand (3′→5′ polymerase activity). A thermostable polymerase that is inactive at a lower temperature outside the above temperature range, but can be activated or re-activated upon exposure to a temperature within the above temperature range is referred to as a heat activation enzyme in the present disclosure. A thermostable polymerase that is inactive at a higher temperature outside the above temperature range, but can be activated or re-activated upon exposure to a temperature within the above temperature range is referred to as a heat inactivation enzyme in the present disclosure.

### 4.3 Primers

According to the present disclosure, a primer is designed to be capable of acting as a point of initiation of synthesis of a primer extension product (i.e., an amplicon) when placed under a suitable condition (e.g., in the presence of nucleotides and an inducing agent such as a DNA polymerase, and at a suitable temperature and pH). In some embodiments, primers are preferably single-stranded for maximum efficiency in amplification, but may alternatively be provided in the form of a double-stranded duplex. In those embodiments where primers are provided as double stranded, the primers can be first treated to separate its strands before being used to produce primer extension products. In some embodiments, a primer is an oligodeoxyribonucleotides. In other embodiments, a primer is oligoribonucleotides.

In some embodiments, a pair of upstream and downstream primers are designed such that they operably define an amplification region or sequence in a target nucleic acid molecule, which means that the primers have sequences configured for specifically hybridizing to the two ends of a region in the target nucleic acid molecule to be amplified. According to the present disclosure, in some embodiments, primers are configured to be substantially complementary to the template strand in a target nucleic acid, which means that the base pairing between the primer and the target is sufficient such that the hybridization is specific and stable for the primer extension reaction to initiate. The percentage of base pairing for two sequences to be considered “substantially complementary” also depends on stringency of the hybridization condition, and the selection of such percentage and condition would be apparent to those skilled in the art upon consideration of the present disclosure.

In some embodiments, before performing the present method, a target sequence is selected. Particularly, in some embodiments, selection of the target sequence is based on determining the genus and species of the organism of interest. In some embodiments, a genomic sequence present in relatively greater abundance in an organism is selected as the target. In some embodiments, a target sequence is selected from a ribosomal RNA (rRNA) encoding gene or a mitochondria gene. In some embodiments, a genomic sequence that is unique to the organism of relevance is selected. For example, to identify a unique sequence of an organism, in some embodiments, a candidate sequence of the organism of interest is compared to sequences of other closely related species in the evolution, such as an ortholog gene in a different species. An ortholog is a gene or genes that are related by vertical descent and are responsible for substantially the same or identical functions in different organisms. For example, mouse epoxide hydrolase and human epoxide hydrolase can be considered orthologs for the biological function of hydrolysis of epoxides. Genes are related by vertical descent when, for example, they share sequence similarity of sufficient amount to indicate they are homologous, or related by evolution from a common ancestor. In some embodiments, a genomic sequence that is conserved across members of a species is selected as the target. In some embodiments, a genomic sequence that is prone to have a genetic mutation of interest is selected as the target.

Hence, in some embodiments, the primer sequence is not completely complementary to the template strand of a target nucleic acid molecule, and the sequence of a primer can be optimized even though the target sequence is determined. For example, in some embodiments, a non-complementary fragment may be attached to the 5′ end of a primer, with the remainder of the primer sequence being complementary to the strand. For example, in other embodiments, a primer contains non-complementary bases or fragments interspersed within regions that are complementary to the target. As would be appreciated by those skilled in the art, variations in the design of primers are possible, as long as the primer has sufficient base pairing with the template strand to be amplified to hybridize therewith, thereby forming a template for the next round of amplification.

Without being bound by the theory, it is contemplated that amplification rate of the accelerated SEA method is affected by at least the following three factors: (1) probability of formation of denaturation bubbles, (2) amplification efficiency of the polymerase, and (3) efficiency of specific binding primers to target sequences in the denaturation bubbles.

Particularly, formation of denaturation bubbles and the amplification efficiency of the polymerase are affected by the reaction temperature (Chander et al. “A novel thermostable polymerase for RNA and DNA loop-mediated isothermal amplification (LAMP),” Front. Microbiol., (2014) 5:395; Sanchez et al. “DNA kinks and bubbles: temperature dependence of the elastic energy of sharply bent 10-nm-size DNA molecules,” Physical Review E (2013) 87: 22710). The dynamic open and close of denaturation bubbles in a double-stranded DNA molecule would become more frequent with the increase of temperature (Adamcik et al., “Quantifying supercoiling-induced denaturation bubbles in DNA,” Soft Matter, (2012) 8: 8651-8658).

Further, the amplification efficiency of a polymerase is also affected by the reaction temperature. Particularly, the reaction temperature under which enzymatic activity reaches the maximum level is referred to as the optimal temperature for that particular enzyme. For example, optimal temperature for Bst DNA polymerase has been reported as 65° C. (Kucera et al. “DNA-dependent DNA polymerases,” Current protocols in molecular biology, (2008) 84: 3-5).

Finally, the efficiency of primer-target binding is affected by the relationship between the reaction temperature and the melting temperature (Tₘ or Tₘ value) of the primer, and the Tₘ in turn depends on the sequence (e.g., the G/C content) of the primer. Typically, when the reaction temperature is in the proximity of the primer's Tₘ, the primer would efficiently bind to its target. Yet, a reaction temperature that is much higher than primer's Tₘ would hinder primer-target binding, and a reaction temperature that is much lower than primer's Tₘ would result in excessive non-specific primer binding and amplification (Kwok et al., “Effects of primer-template mismatches on the polymerase chain reaction: human immunodeficiency virus type 1 model studies,” Nucleic Acids Res., (1990) 18: 999-1005; Alvarez-Fernandez in Methods in enzymology, Elsevier, Editon edn., (2013), vol. 529, pp. 1-21). Methods for designing a primer sequence having a particular Tₘ value and methods for determining the optimal temperature of a given enzyme are known in the art. Further, suitable reaction temperature and primer's Tₘ can be determined using methods known in the art, including but not limited to the exemplary procedure described in Example 6 of the present disclosure (Section 5.7.1).

In some embodiments, the Tₘ value of a primer is within ±5° C. of the optimal temperature of the polymerase. In some embodiments, the Tₘ value of a primer is within ±4° C. of the optimal temperature of the polymerase. In some embodiments, the Tₘ value of a primer is within ±3° C. of the optimal temperature of the polymerase. In some embodiments, the Tₘ value of a primer is within ±2° C. of the optimal temperature of the polymerase. In some embodiments, the Tₘ value of a primer is within ±1° C. of the optimal temperature of the polymerase. In some embodiments, the Tₘ value of a primer is within ±0.5° C. of the optimal temperature of the polymerase.

For example, in specific embodiments where the polymerase is Bst DNA polymerase, the Tₘ value of a primer used for the reaction is selected from the range of about 58° C.-68° C. In various embodiments where the polymerase is Bst DNA polymerase, the Tₘ value of a primer used for the reaction is about 58° C., about 58.5° C., about 59° C., about 59.5° C., about 60° C., about 60.5° C., about 61° C., about 61.5° C., about 62° C., about 62.5° C., about 63° C., about 63.5° C., about 64° C., about 64.5° C., about 65° C., about 65.5° C., about 66° C., about 66.5° C., about 67° C., about 67.5° C., or about 68° C.

In some embodiments, the Tₘ values of the two primers in a primer pair are about the same. In particular embodiments, the Tₘ values of a pair of primers differ from each other by less than about 5%. In particular embodiments, the Tₘ values of a pair of primers differ from each other by less than about 4%. In particular embodiments, the Tₘ values of a pair of primers differ from each other by less than about 3%. In particular embodiments, the Tₘ values of a pair of primers differ from each other by less than about 2%. In particular embodiments, the Tₘ values of a pair of primers differ from each other by less than about 1%. In particular embodiments, the Tₘ values of a pair of primers differ from each other by less than about 0.5%.

In some embodiments, the Tₘ values of the two primers in a primer pair are about the same. In particular embodiments, the Tₘ values of a pair of primers differ from each other by less than about 5° C. In particular embodiments, the Tₘ values of a pair of primers differ from each other by less than about 4° C. In particular embodiments, the Tₘ values of a pair of primers differ from each other by less than about 3° C. In particular embodiments, the Tₘ values of a pair of primers differ from each other by less than about 2° C. In particular embodiments, the Tₘ values of a pair of primers differ from each other by less than about 1° C. In particular embodiments, the Tₘ values of a pair of primers differ from each other by less than about 0.5° C.

Without being bound by theory, it is contemplated that the primers used in the present methods hybridize to target nucleic acid molecules when the target molecule is only partially denatured. Further, it is contemplated that denaturation bubbles dynamically open and close in a target nucleic acid molecule, leaving the time window for specific primer hybridization much shorter than conventional PCR where the target molecule is complete denatured before primer annealing. Furthermore, it is contemplated that a stable hybridization between the primer and the template strand in the target nucleic acid molecule promotes primer extension catalyzed by a polymerase. It is further contemplated that while G-C base pairing typically is more stable, A-T base pairing may occur at a faster rate (Raymaekers et al., “Checklist for optimization and validation of real-time PCR assays,” J. Clin. Lab. Anal., (2009) 23:145-151).

Accordingly, in some embodiments, the primers used in the present methods are designed to have a suitable G/C content. In specific embodiments, the primers have a suitable G/C contents at the end where the polymerase initiates primer extension. For example, in some embodiments, where the polymerase elongates a primer from the primer's 3′ end, the primer can be specifically designed to have a suitable G/C content in the region closer to its 3′ end such that the primer can rapidly form stable hybridization with the template strand. Alternatively, in those embodiments where the polymerase elongates a primer from the primer's 5′ end, the primer can be specifically designed to have a suitable G/C content in the region closer to its 5′ end such that the primer can rapidly form stable hybridization with the template strand. The suitable G/C contents of a primer can be determined using methods known in the art, including but not limited to the exemplary procedure described in Example 6 of the present disclosure (Section 5.7.2).

In specific embodiments where the polymerase initiates primer extension at the 3′ end of the primer, the primer has G or C as the 3′-terminal nucleotide. In some embodiments, the primer comprises a G/C content of about 40% to about 60%. In specific embodiments, the primer comprises a G/C content is about 40%. In specific embodiments, the primer comprises a G/C content is about 45%. In specific embodiments, the primer comprises a G/C content is about 50%. In specific embodiments, the primer comprises a G/C content is about 55%. In specific embodiments, the primer comprises a G/C content is about 60%.

In specific embodiments, the primer comprises a G/C content of at least 40% in a continuous 5-nt region including the 3′ terminal nucleotide. In specific embodiments, the primer comprises a G/C content of at least 40% in a continuous 5-nt region including the 3′ terminal nucleotide, where the 3′ terminal nucleotide is also G or C. In specific embodiments, the primer comprises a G/C content of at least 60% in a continuous 5-nt region including the 3′ terminal nucleotide. In specific embodiments, the primer comprises a G/C content of at least 60% in a continuous 5-nt region including the 3′ terminal nucleotide, where the 3′ terminal nucleotide is also G or C. In specific embodiments, the primer comprises a G/C content of at least 80% in a continuous 5-nt region including the 3′ terminal nucleotide. In specific embodiments, the primer comprises a G/C content of at least 80% in a continuous 5-nt region including the 3′ terminal nucleotide, where the 3′ terminal nucleotide is also G or C. In specific embodiments, the primer comprises a G/C content of 100% in a continuous 5-nt region including the 3′ terminal nucleotide. In specific embodiments, the primer comprises a G/C content of 100% in continuous 5-nt region including the 3′ terminal nucleotide, where the 3′ terminal nucleotide is also G or C.

In specific embodiments where the polymerase initiates primer extension at the 5′ end of the primer, the primer has G or C as the 5′-terminal nucleotide. In some embodiments, the primer comprises a G/C content of about 40% to about 60%. In specific embodiments, the primer comprises a G/C content is about 40%. In specific embodiments, the primer comprises a G/C content is about 45%. In specific embodiments, the primer comprises a G/C content is about 50%. In specific embodiments, the primer comprises a G/C content is about 55%. In specific embodiments, the primer comprises a G/C content is about 60%.

In specific embodiments, the primer comprises a G/C content of at least 40% in a continuous 5-nt region including the 5′ terminal nucleotide. In specific embodiments, the primer comprises a G/C content of at least 40% in a continuous 5-nt region including the 5′ terminal nucleotide, where the 5′ terminal nucleotide is also G or C. In specific embodiments, the primer comprises a G/C content of at least 60% in a continuous 5-nt region including the 5′ terminal nucleotide. In specific embodiments, the primer comprises a G/C content of at least 60% in a continuous 5-nt region including the 5′ terminal nucleotide, where the 5′ terminal nucleotide is also G or C. In specific embodiments, the primer comprises a G/C content of at least 80% in a continuous 5-nt region including the 5′ terminal nucleotide. In specific embodiments, the primer comprises a G/C content of at least 80% in a continuous 5-nt region including the 5′ terminal nucleotide, where the 5′ terminal nucleotide is also G or C. In specific embodiments, the primer comprises a G/C content of 100% in a continuous 5-nt region including the 5′ terminal nucleotide. In specific embodiments, the primer comprises a G/C content of 100% in continuous 5-nt region including the 5′ terminal nucleotide, where the 5′ terminal nucleotide is also G or C.

Without being bound by the theory, it is contemplated that a primer having a sequence capable of forming self-complementary secondary structures, or a pair of primers having complementary sequences with respect to each other, can hinder amplification reaction using such primers (Meagher et al., “Impact of primer dimers and self-amplifying hairpins on reverse transcription loop-mediated isothermal amplification detection of viral RNA,” Analyst, (2018) 143:1924-1933). Accordingly, in some embodiments, after the selection of the target sequence for primer hybridization, the primer sequence can be further optimized to avoid or reduce the possibility of forming these intra-primer or inter-primer complementary structures. Methods for primer sequence optimization are known in the art, including but not limited to the exemplary procedure described in Example 6 of the present disclosure (Section 5.7.3).

According to the present disclosure, selection of the length of a primer can depend on various factors, including but not limited to the amplification reaction temperature and time. Without being limited by the theory, it is contemplated that the higher reaction temperature is, the longer complementary region between the primer and the target would be used to avoid non-specific amplification.

Without being bound by the theory, it is further contemplated that reducing the primer extension time in each amplification cycle can significantly reduce the total time needed for producing the amplicon at a detectable amount, thereby reducing time needed for detecting the presence of a target and related diagnosis. Hence, in some embodiments, the primer is configure for specifically hybridizing to a substantial portion of the amplification region, such that the number of nucleotides to be elongated in each amplification cycle (e.g., the length difference between the amplicon and the primer) is relatively small.

For example, in specific embodiments, the ratio between the length of a primer and the total length of the amplicon is in the range of about 30% to about 80%. In specific embodiments, the ratio between the length of a primer and the total length of the amplicon is in the range of about 30%. In specific embodiments, the ratio between the length of a primer and the total length of the amplicon is in the range of about 35%. In specific embodiments, the ratio between the length of a primer and the total length of the amplicon is in the range of about 40%. In specific embodiments, the ratio between the length of a primer and the total length of the amplicon is in the range of about 45%. In specific embodiments, the ratio between the length of a primer and the total length of the amplicon is in the range of about 50%. In specific embodiments, the ratio between the length of a primer and the total length of the amplicon is in the range of about 55%. In specific embodiments, the ratio between the length of a primer and the total length of the amplicon is in the range of about 60%. In specific embodiments, the ratio between the length of a primer and the total length of the amplicon is in the range of about 65%. In specific embodiments, the ratio between the length of a primer and the total length of the amplicon is in the range of about 70%. In specific embodiments, the ratio between the length of a primer and the total length of the amplicon is in the range of about 75%. In specific embodiments, the ratio between the length of a primer and the total length of the amplicon is in the range of about 80%.

In some embodiments, a pair of primers are configured to define an amplification region that is relatively short, yet having a unique sequence indicative of the identity, status, origin or source of the target nucleic acid. In this respect, the selection of the amplification region in a target nucleic acid depends on the purpose of detection or the scenario of application, and would become apparent to those skilled in the art upon consideration of the present disclosure. For example, to detect the presence or absence of genetic mutation or polymorphism in a sample, the amplification region can be selected to include the expected site of the mutation or polymorphism. To detect the presence of a pathological microorganism in a sample, the amplification region can be selected to cover a known signature sequence in the genome of the microorganism.

In some embodiments, the pair of primers are configured to amplify a region in a target nucleic acid molecule that is less than 100 bp long. In some embodiments, the amplicon produced by the present method is less than 90 bp long. In some embodiments, the amplicon produced by the present method is less than 80 bp long. In some embodiments, the amplicon produced by the present method is less than 70 bp long. In some embodiments, the amplicon produced by the present method is less than 60 bp long. In some embodiments, the amplicon produced by the present method is less than 50 bp long. In some embodiments, the amplicon produced by the present method is about 20-50 bp long. In some embodiments, the amplicon produced by the present method is about 30-50 bp long. In some embodiments, the amplicon produced by the present method is about 35-50 bp long.

In some embodiments, to reduce time needed for primer extension, the pair of primers are configured to produce a short amplicon of about 20 base pair (bp) to about 50 bp in length. The amplicon comprises at least a central portion that corresponds to an unique sequence in the target nucleic acid molecule, which central portion may be flanked by primer sequences that are either the same as or different from sequences in the target molecule. For example, in specific embodiments, the amplicon is about 20 bp in length. In specific embodiments, the amplicon is about 21 bp in length. In specific embodiments, the amplicon is about 22 bp in length. In specific embodiments, the amplicon is about 23 bp in length. In specific embodiments, the amplicon is about 24 bp in length. In specific embodiments, the amplicon is about 25 bp in length. In specific embodiments, the amplicon is about 25 bp in length. In specific embodiments, the amplicon is about 26 bp in length. In specific embodiments, the amplicon is about 27 bp in length. In specific embodiments, the amplicon is about 28 bp in length. In specific embodiments, the amplicon is about 29 bp in length. In specific embodiments, the amplicon is about 30 bp in length. In specific embodiments, the amplicon is about 31 bp in length. In specific embodiments, the amplicon is about 32 bp in length. In specific embodiments, the amplicon is about 33 bp in length. In specific embodiments, the amplicon is about 34 bp in length. In specific embodiments, the amplicon is about 35 bp in length. In specific embodiments, the amplicon is about 36 bp in length. In specific embodiments, the amplicon is about 37 bp in length. In specific embodiments, the amplicon is about 38 bp in length. In specific embodiments, the amplicon is about 39 bp in length. In specific embodiments, the amplicon is about 40 bp in length. In specific embodiments, the amplicon is about 41 bp in length. In specific embodiments, the amplicon is about 42 bp in length. In specific embodiments, the amplicon is about 43 bp in length. In specific embodiments, the amplicon is about 44 bp in length. In specific embodiments, the amplicon is about 45 bp in length. In specific embodiments, the amplicon is about 46 bp in length. In specific embodiments, the amplicon is about 47 bp in length. In specific embodiments, the amplicon is about 48 bp in length. In specific embodiments, the amplicon is about 49 bp in length. In specific embodiments, the amplicon is about 50 bp in length.

In some embodiments, to reduce time needed for primer extension, the primer is configured to specifically hybridize to a substantial portion of the amplified region in the target molecule. Specifically, in some embodiments where the amplicon is about 20 to 50 bp long, at least one of the pair of primers is about 10 to 25 nucleotides (nt) in length. In some embodiments, where the amplicon is about 20 to 50 bp long, both primers in the primer pair are about 10 to 25 nt in length.

Specifically, in some embodiments where the amplicon is about 20 to 50 bp, at least one of the pair of primers is about 10 nt in length. In some embodiments where the amplicon is about 20 to 50 bp long, at least one of the pair of primers is about 11 nt in length. In some embodiments where the amplicon is about 20 to 50 bp long, at least one of the pair of primers is about 12 nt in length. In some embodiments where the amplicon is about 20 to 50 bp long, at least one of the pair of primers is about 13 nt in length. In some embodiments where the amplicon is about 20 to 50 bp long, at least one of the pair of primers is about 14 nt in length. In some embodiments where the amplicon is about 20 to 50 bp long, at least one of the pair of primers is about 15 nt in length. In some embodiments where the amplicon is about 20 to 50 bp long, at least one of the pair of primers is about 16 nt in length. In some embodiments where the amplicon is about 35 to 50 bp long, at least one of the pair of primers is about 17 nt in length. In some embodiments where the amplicon is about 20 to 50 bp long, at least one of the pair of primers is about 18 nt in length. In some embodiments where the amplicon is about 35 to 50 bp long, at least one of the pair of primers is about 19 nt in length. In some embodiments where the amplicon is about 20 to 50 bp long, at least one of the pair of primers is about 20 nt in length. In some embodiments where the amplicon is about 35 to 50 bp long, at least one of the pair of primers is about 21 nt in length. In some embodiments where the amplicon is about 20 to 50 bp long, at least one of the pair of primers is about 22 nt in length. In some embodiments where the amplicon is about 35 to 50 bp long, at least one of the pair of primers is about 23 nt in length. In some embodiments where the amplicon is about 20 to 50 bp long, at least one of the pair of primers is about 24 nt in length. In some embodiments where the amplicon is about 35 to 50 bp long, at least one of the pair of primers is about 25 nt in length. In some embodiments where the amplicon is about 20 to 50 bp long, at least one of the pair of primers is about 26 nt in length. In some embodiments where the amplicon is about 35 to 50 bp long, at least one of the pair of primers is about 27 nt in length. In some embodiments where the amplicon is about 20 to 50 bp long, at least one of the pair of primers is about 28 nt in length. In some embodiments where the amplicon is about 35 to 50 bp long, at least one of the pair of primers is about 29 nt in length. In some embodiments where the amplicon is about 20 to 50 bp long, at least one of the pair of primers is about 30 nt in length.

As would be appreciated by those of ordinary skill in the art, during actual primer design processes, selections of a primer sequence based on different considerations (e.g., Tm, G/C content, sequence complementarity) may contradict with one another. Accordingly, in some embodiments, the different considerations can be compared with one another to determine an order of priority among different consideration. Particularly, when a selection of primer sequence based on a lower-priority consideration contradicts with a selection of primer sequence based on a higher-priority consideration, the selection based on the higher-priority consideration can be beneficially adopted. Example 6 further provides exemplary process for determining such order of priority in Section 5.7.4.

### 4.4 Enzymes

According to the present disclosure, polymerases that can be used in connection with the present methods include thermostable polymerases having strand displacement activity in the temperature range of about 50-80° C. In some embodiments, the thermostable polymerase selected to be used in connection with the present method has the strand displacement activity in the temperature of about 70-80° C. In some embodiments, the thermostable polymerases has 5′→3′ polymerase activity and is capable of elongating a primer annealed to a template strand starting at the 3′ end of the primer towards the 5′ end of the template strand, thereby displacing the original complementary strand in the 5′→3′ direction with respect to the original complementary strand. In other embodiments, thermostable polymerases has 3′→5′ polymerase activity and is capable of elongating a primer annealed to a template strand starting at the 5′ end of the primer towards the 3′ end of the template strand, thereby displacing the original complementary strand in the 3′→5′ direction with respect to the original complementary strand.

In contrast to strand displacement, some polymerases, such as Taq DNA polymerase, degrade an encountered downstream complementary strand via an exonuclease activity. Although the outcome is also the formation of a duplex having the original template strand and the newly synthesized complementary strand (with the original complementary strand removed by degradation), the exonuclease activity can reduce the total amount of nucleic acid fragments that the reaction aims to amplify, and hence is less ideal in some (but not all) scenarios of applications. Hence, certain polymerases having been engineered to remove the 5′→3′ exonuclease activity of the wild-type enzyme, while the polymerase activity and strand displacement activity are retained. Accordingly, in some embodiments, the thermostable polymerases has 5′→3′ polymerase activity, and is devoid of 5′→3′ exonuclease activity. In some embodiments, the thermostable polymerases has 3′→5′ polymerase activity, and is devoid of 3′→5 exonuclease activity.

In some embodiments, the thermostable polymerase is a heat activation enzyme. In some embodiments, the thermostable polymerase is a heat inactivation enzyme. In some embodiments, the thermostable polymerase has reverse transcriptase activity. In some embodiments, the thermostable polymerase has an amplification speed of at least 10 nt/s at its optimal temperature.

Examples of thermostable polymerases that can be used in connection with the present disclosure include but are not limited to phi29 DNA polymerase or a truncated or mutated version thereof, DNA Polymerase I, or a truncated or mutated version thereof, Vent® DNA Polymerase or a truncated or mutated version thereof, and Bacillus stearothermophilus (Bst) DNA Polymerase or a truncated or mutated version thereof, and Thermus aquaticus (Taq) DNA polymerase or a truncated or mutated version thereof.

In some embodiments, the polymerase is Bst DNA polymerase. In some embodiments, the polymerase is full-length Bst DNA polymerase. In some embodiments, the polymerase is Bst DNA polymerase, Large Fragment. In some embodiments, the polymerase is a mutated version of Bst DNA polymerase. In particularly embodiments, the mutated Bst DNA polymerase is devoid of the 5′→3′ exonuclease activity. In some embodiments, the Bst DNA polymerase is commercially available. In specific embodiments, the Bst DNA polymerase is selected from Bst 2.0 DNA Polymerase, Bst 2.0 WarmStart DNA Polymerase and Bst 3.0 DNA Polymerase commercially available from New England BioLabs®.

In some embodiments, the polymerase is DNA polymerase I or a mutated or truncated version thereof. In some embodiments, the polymerase is wild-type DNA polymerase I, large (Klenow) fragment. In some embodiments, the polymerase is Klenow exo⁻. In some embodiments, the polymerase is phi29 DNA polymerase or a mutated or truncated version thereof. In some embodiments, the polymerase is Vent® DNA Polymerase, Deep Vent® (exo-) DNA Polymerase, Deep Vent® DNA Polymerase, or Vent® (exo-)DNA Polymerase.

In some embodiments, the polymerase is Taq DNA polymerase. In some embodiments, the polymerase is a mutated version of Taq DNA polymerase. In some embodiments, the Taq DNA polymerase is a heat activation enzyme. In some embodiments, the Taq DNA polymerase is commercially available. In specific embodiments, the Taq DNA polymerase is selected from Hot Start Taq DNA Polymerase, EpiMark® Hot Start Taq DNA Polymerase, OneTaq® DNA Polymerase, OneTaq® Hot Start DNA Polymerase and LongAmp® Taq DNA Polymerase commercially available from New England BioLabs®. In some embodiments, the Taq DNA polymerase is LongTaq DNA Polymerase.

While the above exemplary enzymes or commercial products can be used in connection with the present methods and kits, they by no means limit the present disclosure. As would be understood by those skilled in the art, other enzymes currently known or to be discovered in the future that satisfy the description of desirable polymerase activities as disclosed herein are also contemplated by and included in the present disclosure. Additional polymerases suitable for use in connection with the present disclosure can be generated and selected using methods known in the art. For example, a wild-type polymerase can be mutated via directed mutagenesis or random mutagenesis to produce peptide variants that can be subsequently screened (e.g., individually or via a high-throughput assay) to identify mutants having desirable polymerase activities. For illustration purposes, several exemplary methods useful for enzyme mutagenesis and evolution are provided below.

Directed evolution is a powerful approach that involves the introduction of mutations targeted to a specific gene or an oligonucleotide sequence containing a gene in order to improve and/or alter the properties or production of an enzyme, protein or peptide (e.g., a polymerase and specifically DNA polymerase). Improved and/or altered enzymes, proteins or peptides can be identified through the development and implementation of sensitive high-throughput assays that allow automated screening of many enzyme or peptide variants (for example, >1.0×10⁴). Iterative rounds of mutagenesis and screening typically are performed to afford an enzyme or peptide with optimized properties. Computational algorithms that can help to identify areas of the gene for mutagenesis also have been developed and can significantly reduce the number of enzyme or peptide variants that need to be generated and screened (See: Fox, R. J., et al., Trends Biotechnol., 2008, 26, 132-138; Fox, R. J., et al., Nature Biotechnol., 2007, 25, 338-344). Numerous directed evolution technologies have been developed and shown to be effective at creating diverse variant libraries, and these methods have been successfully applied to the improvement of a wide range of properties across many enzyme and protein classes (for reviews, see: Hibbert et al., Biomol. Eng., 2005, 22, 11-19; Huisman and Lalonde, In Biocatalysis in the pharmaceutical and biotechnology industries, pgs. 717-742 (2007), Patel (ed.), CRC Press; Often and Quax, Biomol. Eng., 2005, 22, 1-9; and Sen et al., Appl. Biochem. Biotechnol., 2007, 143, 212-223). Enzyme and protein characteristics that have been improved and/or altered by directed evolution technologies include, for example: temperature stability, for robust high temperature processing; pH stability, for bioprocessing under lower or higher pH conditions; substrate or product tolerance, so that high product titers can be achieved; binding (Kₘ), including broadening of ligand or substrate binding to include non-natural substrates; inhibition (IQ, to remove inhibition by products, substrates, or key intermediates; activity (keg), to increase enzymatic reaction rates to achieve desired flux; isoelectric point (pI) to improve protein or peptide solubility; acid dissociation (pKa) to vary the ionization state of the protein or peptide with respect to pH.

A number of exemplary methods have been developed for the mutagenesis and diversification of genes and oligonucleotides to introduce desired properties into specific enzymes, proteins and peptides. Such methods are well known to those skilled in the art. Any of these can be used to alter and/or optimize the activity of an enzyme, protein, or peptide, including a polymerase such as DNA polymerase. Such methods include, but are not limited to error-prone polymerase chain reaction (EpPCR), which introduces random point mutations by reducing the fidelity of DNA polymerase in PCR reactions (See: Pritchard et al., J. Theor. Biol., 2005, 234:497-509); Error-prone Rolling Circle Amplification (epRCA), which is similar to epPCR except a whole circular plasmid is used as the template and random 6-mers with exonuclease resistant thiophosphate linkages on the last 2 nucleotides are used to amplify the plasmid followed by transformation into cells in which the plasmid is re-circularized at tandem repeats (Fujii et al., Nucleic Acids Res., 2004, 32:e145; and Fujii et al., Nat. Protoc., 2006, 1, 2493-2497); DNA, Gene, or Family Shuffling, which typically involves digestion of two or more variant genes with nucleases such as Dnase I or EndoV to generate a pool of random fragments that are reassembled by cycles of annealing and extension in the presence of DNA polymerase to create a library of chimeric genes (Stemmer, Proc. Natl. Acad. Sci. U.S.A., 1994, 91, 10747-10751; and Stemmer, Nature, 1994, 370, 389-391); Staggered Extension (StEP), which entails template priming followed by repeated cycles of 2-step PCR with denaturation and very short duration of annealing/extension (as short as 5 sec) (Zhao et al., Nat. Biotechnol., 1998, 16, 258-261); Random Priming Recombination (RPR), in which random sequence primers are used to generate many short DNA fragments complementary to different segments of the template (Shao et al., Nucleic Acids Res., 1998, 26, 681-683).

Additional methods include Heteroduplex Recombination, in which linearized plasmid DNA is used to form heteroduplexes that are repaired by mismatch repair (See: Volkov et al, Nucleic Acids Res., 1999, 27:e18; Volkov et al., Methods Enzymol., 2000, 328, 456-463); Random Chimeragenesis on Transient Templates (RACHITT), which employs Dnase I fragmentation and size fractionation of single-stranded DNA (ssDNA) (See: Coco et al., Nat. Biotechnol., 2001, 19, 354-359); Recombined Extension on Truncated Templates (RETT), which entails template switching of unidirectionally growing strands from primers in the presence of unidirectional ssDNA fragments used as a pool of templates (See: Lee et al., J. Mol. Cat., 2003, 26, 119-129); Degenerate Oligonucleotide Gene Shuffling (DOGS), in which degenerate primers are used to control recombination between molecules; (Bergquist and Gibbs, Methods Mol. Biol., 2007, 352, 191-204; Bergquist et al., Biomol. Eng., 2005, 22, 63-72; Gibbs et al., Gene, 2001, 271, 13-20); Incremental Truncation for the Creation of Hybrid Enzymes (ITCHY), which creates a combinatorial library with 1 base pair deletions of a gene or gene fragment of interest (See: Ostermeier et al., Proc. Natl. Acad. Sci. U.S.A., 1999, 96, 3562-3567; and Ostermeier et al., Nat. Biotechnol., 1999, 17, 1205-1209); Thio-Incremental Truncation for the Creation of Hybrid Enzymes (THIO-ITCHY), which is similar to ITCHY except that phosphothioate dNTPs are used to generate truncations (See: Lutz et al., Nucleic Acids Res., 2001, 29, E16); SCRATCHY, which combines two methods for recombining genes, ITCHY and DNA Shuffling (See: Lutz et al., Proc. Natl. Acad. Sci. U.S.A., 2001, 98, 11248-11253); Random Drift Mutagenesis (RNDM), in which mutations made via epPCR are followed by screening/selection for those retaining usable activity (See: Bergquist et al., Biomol. Eng., 2005, 22, 63-72); Sequence Saturation Mutagenesis (SeSaM), a random mutagenesis method that generates a pool of random length fragments using random incorporation of a phosphothioate nucleotide and cleavage, which is used as a template to extend in the presence of “universal” bases such as inosine, and replication of an inosine-containing complement gives random base incorporation and, consequently, mutagenesis (See: Wong et al., Biotechnol. J., 2008, 3, 74-82; Wong et al., Nucleic Acids Res., 2004, 32, e26; Wong et al., Anal. Biochem., 2005, 341, 187-189); Synthetic Shuffling, which uses overlapping oligonucleotides designed to encode “all genetic diversity in targets” and allows a very high diversity for the shuffled progeny (See: Ness et al., Nat. Biotechnol., 2002, 20, 1251-1255); Nucleotide Exchange and Excision Technology NexT, which exploits a combination of dUTPs incorporation followed by treatment with uracil DNA-glycosylase and then piperidine to perform endpoint DNA fragmentation (See: Muller et al., Nucleic Acids Res., 33:e117).

Further mutagenesis methods include Sequence Homology-Independent Protein Recombination (SHIPREC), in which a linker is used to facilitate fusion between two distantly related or unrelated genes, and a range of chimeras is generated between the two genes, resulting in libraries of single-crossover hybrids (See: Sieber et al., Nat. Biotechnol., 2001, 19, 456-460); Gene Site Saturation Mutagenesis™ (GSSM™), in which the starting materials include a supercoiled double stranded DNA (dsDNA) plasmid containing an insert and two primers which are degenerate at the desired site of mutations, enabling all amino acid variations to be introduced individually at each position of a protein or peptide (See: Kretz et al., Methods Enzymol., 2004, 388, 3-11); Combinatorial Cassette Mutagenesis (CCM), which involves the use of short oligonucleotide cassettes to replace limited regions with a large number of possible amino acid sequence alterations (See: Reidhaar-Olson et al. Methods Enzymol., 1991, 208, 564-586; Reidhaar-Olson et al. Science, 1988, 241, 53-57); Combinatorial Multiple Cassette Mutagenesis (CMCM), which is essentially similar to CCM and uses epPCR at high mutation rate to identify hot spots and hot regions and then extension by CMCM to cover a defined region of protein sequence space (See: Reetz et al., Angew. Chem. Int. Ed Engl., 2001, 40, 3589-3591); the Mutator Strains technique, in which conditional is mutator plasmids, utilizing the mutD5 gene, which encodes a mutant subunit of DNA polymerase III, to allow increases of 20 to 4000× in random and natural mutation frequency during selection and block accumulation of deleterious mutations when selection is not required (See: Selifonova et al., Appl. Environ. Microbiol., 2001, 67, 3645-3649); Low et al., J. Mol. Biol., 1996, 260, 3659-3680).

Additional exemplary methods include Look-Through Mutagenesis (LTM), which is a multidimensional mutagenesis method that assesses and optimizes combinatorial mutations of a selected set of amino acids (See: Rajpal et al., Proc. Natl. Acad. Sci. U.S.A., 2005, 102, 8466-8471); Gene Reassembly, which is a homology-independent DNA shuffling method that can be applied to multiple genes at one time or to create a large library of chimeras (multiple mutations) of a single gene (See: Short, J. M., U.S. Pat. No. 5,965,408, Tunable GeneReassembly™); in Silico Protein Design Automation (PDA), which is an optimization algorithm that anchors the structurally defined protein backbone possessing a particular fold, and searches sequence space for amino acid substitutions that can stabilize the fold and overall protein energetics, and generally works most effectively on proteins with known three-dimensional structures (See: Hayes et al., Proc. Natl. Acad. Sci. U.S.A., 2002, 99, 15926-15931); and Iterative Saturation Mutagenesis (ISM), which involves using knowledge of structure/function to choose a likely site for enzyme improvement, performing saturation mutagenesis at chosen site using a mutagenesis method such as Stratagene QuikChange (Stratagene; San Diego Calif.), screening/selecting for desired properties, and, using improved clone(s), starting over at another site and continue repeating until a desired activity is achieved (See: Reetz et al., Nat. Protoc., 2007, 2, 891-903; Reetz et al., Angew. Chem. Int. Ed Engl., 2006, 45, 7745-7751).

In addition to biological methods described above, the evolution of enzymes (e.g., polymerases) also can be conducted using chemical synthesis methods. For example, large combinatorial peptide libraries (e.g., >1.0×10⁶ members) containing mutational variants can be synthesized by using known solution phase or solid phase peptide synthesis technologies (See review: Shin, D.-S., et al., J. Biochem. Mol. Bio., 2005, 38, 517-525). Chemical peptide synthesis methods can be used to produce polymerase variants containing a wide range of alpha-amino acids, including the natural proteinogenic amino acids, as well as non-natural and/or non-proteinogenic amino acids, such as amino acids with non-proteinogenic side chains, or alternatively D-amino acids, or alternatively beta-amino acids.

Any of the aforementioned methods for enzyme mutagenesis can be used alone or in any combination to improve the performance of the enzymes, proteins, and peptides. Similarly, any of the aforementioned methods for mutagenesis and/or display can be used alone or in any combination to enable the creation of polymerase variants which may be selected for improved properties.

In some embodiments, the mutated polymerase has a nucleic acid sequence that is at least 80% identical to the sequence of the wild-type counterpart. In some embodiments, the mutated polymerase has a nucleic acid sequence that is at least 85% identical to the sequence of the wild-type counterpart. In some embodiments, the mutated polymerase has a nucleic acid sequence that is at least 90% identical to the sequence of the wild-type counterpart. In some embodiments, the mutated polymerase has a nucleic acid sequence that is at least 95% identical to the sequence of the wild-type counterpart. In some embodiments, the mutated polymerase has a nucleic acid sequence that is at least 96% identical to the sequence of the wild-type counterpart. In some embodiments, the mutated polymerase has a nucleic acid sequence that is about 97% identical to the sequence of the wild-type counterpart. In some embodiments, the mutated polymerase has a nucleic acid sequence that is about 98% identical to the sequence of the wild-type counterpart. In some embodiments, the mutated polymerase has a nucleic acid sequence that is about 99% identical to the sequence of the wild-type counterpart.

Methods for determining sequence identity are known in the art. For example, inspection of nucleic acid or amino acid sequences for two polypeptides will reveal sequence identity and similarities between the compared sequences. Algorithms well known to those skilled in the art, such as Align, BLAST, Clustal W and others compare and determine a raw sequence similarity or identity, and also determine the presence or significance of gaps in the sequence which can be assigned a weight or score. Such algorithms also are known in the art and are similarly applicable for determining nucleotide sequence similarity or identity. Parameters for sufficient similarity to determine relatedness are computed based on well known methods for calculating statistical similarity, or the chance of finding a similar match in a random polypeptide, and the significance of the match determined. A computer comparison of two or more sequences can, if desired, also be optimized visually by those skilled in the art.

Exemplary parameters for determining relatedness of two or more sequences using the BLAST algorithm, for example, can be as set forth below. Briefly, amino acid sequence alignments can be performed using BLASTP version 2.0.8 (Jan. 5, 1999) and the following parameters: Matrix: 0 BLOSUM62; gap open: 11; gap extension: 1; x_dropoff: 50; expect: 10.0; wordsize: 3; filter: on. Nucleic acid sequence alignments can be performed using BLASTN version 2.0.6 (Sep. 16, 1998) and the following parameters: Match: 1; mismatch: −2; gap open: 5; gap extension: 2; x_dropoff: 50; expect: 10.0; wordsize: 11; filter: off. Those skilled in the art will know what modifications can be made to the above parameters to either increase or decrease the stringency of the comparison, for example, and determine the relatedness of two or more sequences.

In some embodiments, a functional variant of a protein comprises one or more conservative substitutions as compared to the wild-type counterpart. In some embodiments, a functional variant of a protein comprises one or more amino acid residues replaced by non-naturally occurring amino acid residues as compared to the wild-type counterpart.

Wild-type and mutated enzymes (e.g., polymerase) can be screened to select those having desirable properties to be used in connection with the present method. In some embodiments, the enzymes and/or mutated variants are screened for those that retain at least the DNA polymerase activity and strand displacement activity. In some embodiments, the enzymes and/or mutated variants are screened for those that are thermally stable and active in the temperature range of about 50-80° C. In some embodiments, the enzymes and/or mutated variants are screened for those that are thermally stable and active in the temperature range of about 70-80° C. In some embodiments, the enzymes and/or mutated variants are screened for those that have optimal temperature in the temperature range of 50-80° C. In some embodiments, the enzymes and/or mutated variants are screened for those that have optimal temperature in the temperature range of 70-80° C. In some embodiments, the enzymes and/or mutated variants are screened for those that have an elongate speed of at least 10 nt/s at an optimal temperature in the range of 50-80° C. In some embodiments, the enzymes and/or mutated variants are screened for those that have a reverse transcriptase activity. In some embodiments, the enzymes and/or mutated variants are screened for those that are devoid of an exonuclease activity. In some embodiments, the enzymes and/or mutated variants are screened for those that are heat activation and/or heat inactivation enzymes.

The screening can be performed using methods and assays known in the art. For example, whether a given polymerase has strand displacement activity can be determined using strand displacement amplification (SDA) assays such as those described or used in Walker et al. Nucleic Acids Res. 1992 Apr. 11; 20(7): 1691-1696; and Guo et al., Nucleic Acids Res., 2009 Feb. 1; 37, e20. A given enzyme's thermal dynamic properties, including its optimal temperature and elongation speed, can be determined using assays such as those described or used in Rychlik et al., Nucleic Acids Res., 1990 Nov. 21; 18(21), 6409-6412. Whether a polymerase has reverse transcriptase activity can be determined using assays such as those described or used in Shi et al., J. Am. Chem. Soc., 2015 Oct. 16; 137(43), 13804-13806; and Lanford et al., J. Virol., 1995 Apr. 21; 69(7), 4431-4439. Whether a polymerase has exonuclease activity can be determined using assays such as those described or used in Holland et al., P. Natl. Acad. Sci. USA, 1991 Aug. 15; 88(16), 7276-7280; and Beese et al., EMBO J., 1991 Jan. 1; 10(1), 25-33.

A rapid way to screen large libraries of diverse mutated enzyme, protein or peptide variants involves the use of display technologies (For a review, see: Ullman, C G, et al., Briefings Functional Genomics, 2011, 10, 125-134). Peptide display technologies offer the benefit that specific peptide encoding information (e.g., RNA or DNA sequence information) is linked to, or otherwise associated with, each corresponding peptide in a library, and this information is accessible and readable (e.g., by amplifying and sequencing the attached DNA oligonucleotide) after a screening event, thus enabling identification of the individual peptides within a large library that exhibit desirable properties (e.g., high binding affinity). Enzyme peptide mutants that exhibit the desired improved properties (hits) may be subjected to additional rounds of mutagenesis to allow creation of highly optimized enzyme variants.

### 4.5 Methods

In one aspect of the present disclosure, provided herein are methods for the amplification and detection of a target nucleic acid in a sample. The currently methods significantly improve the existing technology based on denaturation bubble-mediated strand exchange amplification (SEA), which was first reported in 2016 by Shi et al (Shi et al. “Triggered isothermal PCR by denaturation bubble-mediated strand exchange amplification” Chem Commun (Camb) (2016) 4; 52(77):11551-4).

Shi et al. reported an SEA assay that employs a Bst DNA polymerase and a pair of specific primers to carry out exponential DNA amplification under an isothermal condition. The isothermal SEA method is based on the spontaneous formation of denatured regions (“bubbles”) in double-stranded DNA (dsDNA) due to ambient thermal fluctuations. A pair of oligonucleotide primers then invade a denaturation bubble, binding to unwound single-stranded DNA in the bubble, extending and replacing the original complementary strand under the action of a polymerase to produce the amplicon. Hence, the method, which utilizes small denaturation bubbles spontaneously formed without heating up the sample, is thought to advantageously eliminate the need for a thermal cycler, and performs the PCR reaction under one temperature typically selected for optimal polymerase activity. Shi et al. 2016 (Supra).

Since its establishment, the isothermal SEA method has been demonstrated and used for rapid detection and diagnosis of various pathogens, such as L. monocytoenes (Zhang et al. “Rapid detection of foodborne pathogen Listeria monocytogenes by strand exchange amplification,” Analytical Biochemistry, (2018) 545:38-42); M. pneumonia (Shi et al. “Rapid diagnosis of Mycoplasma pneumonia infection by denaturation bubble-mediated strand exchange amplification: comparison with LAMP and real-time PCR,” Scientific Reports, vol. 9; article number: 896 (2019)); S. aureus (Liu et al., “Rapid and Simple Detection of Viable Foodborne Pathogen Staphylococcus aureus,” Front Chem. (2019) March 12; 7:124); E. coli (Chinese Patent Application Publication No.: CN 105176971A); B. xylophilus (Liu et al., “The Rapid detection of the Bursaphelenchus Xylophilus by Denaturation Bubble-mediated Strand Exchange Amplification,” Anal. Sci. 2019, 18P-461P.”); and meat source and adulteration (Liu et al., “A simple isothermal nucleic acid amplification method for the effective on-site identification for adulteration of pork source in mutton,” Food Control, 2019, 98 297-302). Capability of the isothermal SEA method in detecting a trace amount of target nucleic acid in a sample (at a concentration as low as 1.0×10⁻¹⁴ M) has also been demonstrated (Chinese Patent Application Publication No.: CN 109136337 A). Additionally, it was discovered that Bst DNA polymerase has an intrinsic reverse transcriptase activity (Shi et al. “Innate reverse transcriptase activity of DNA polymerase for isothermal RNA direct detection.” J. Am. Chem. Soc. (2015) 137, 13804-13806), and isothermal SEA assay employing the Bst DNA polymerase has been shown to efficiently amplify and detect target RNA molecules in a sample without the need for another reverse transcriptase (Chinese Patent Application Publication No.: CN 105176971A).

The present disclosure is based, at least partially, on the surprising discovery that modifying the isothermal SEA method by swiftly changing the reaction temperature, even within a small range of a few degrees, significantly increases the efficiency and speed of amplification by thousands of folds. The present method is hence referred to as “accelerated SEA” in certain passages of this application. Without being bound by the theory, the present disclosure contemplates that formation of denaturation bubbles can be promoted by inducing temperature fluctuations within a small temperature range, which makes it more efficient for primer invading and hybridization. Further, because the temperature fluctuation is within a small range around the optimal temperature the polymerase to catalyze primer elongation, the increased denaturation is not achieved at the expense of elongation speed. Accordingly, in various embodiments, the induced temperature fluctuation is within about 1° C. to about 15° C. of the optimal elongation temperature of a polymerase. In various embodiments, the range of temperature fluctuation used in the present method is less than about 30° C. In various embodiments, the range of temperature fluctuation used in the present method is less than about 25° C. In various embodiments, the range of temperature fluctuation used in the present method is less than about 20° C.

In some embodiments, the present method comprises contacting a polymerase and a pair of specific oligonucleotide primers with a sample containing or suspected of containing a target nucleic acid, thereby forming an amplification mixture. The method further comprises subjecting the amplification mixture to a number of thermal cycles between a first temperature and a second temperature, in order to amplify, via the polymerase chain reaction (PCR), a sequence of the target nucleic acid. The production of the amplicon can then be detected, which detection can serve as the basis for various analysis and diagnosis.

According to the present disclosure, at least one of the first and second temperature is suitable for (a) the formation of denaturation bubbles in a double-stranded target molecule; (b) the primers to specifically hybridize to the target nucleic acid; (c) the polymerase to catalyze primer extension in the amplification mixture; or any combination of (a) to (c). In some embodiments, the temperature range between the first and second temperatures is suitable for (a) the formation of denaturation bubbles in a double-stranded target molecule; (b) the primers to specifically hybridize to the target nucleic acid; (c) the polymerase to catalyze primer extension in the amplification mixture; or any combination of (a) to (c).

In some embodiments, the selection of the first or second temperature is based on the type of polymerase used for the amplification. In some embodiments, the second temperature is selected in the proximity of the optimal temperature of the polymerase used. Methods for determining an enzyme's optimal temperature are known in the art. For example, to determine the optimal temperature for a given polymerase to catalyze primer extension under a given condition, a plurality of aliquots of an amplification mixture can be made, each containing the polymerase of interest, same primers, targets, and other reactants at the same concentrations; then the aliquots can be subjected different temperature conditions to perform PCR, and the optimal temperature can be determined by comparing the speed of amplification, such as using real-time PCR monitoring. Further, the optimal elongation temperature of a polymerase can be determined based on reports in the field or suggestions by manufacturers of commercial polymerases.

In some embodiments, the second temperature is selected from the range of ±6° C. of the polymerase's optimal temperature. For illustration and example only, if the optimal elongation temperature of a polymerase is 65° C., then in some embodiments, the second temperature can be selected from the range of about 59-71° C. Specifically in this example, the second temperature can be about 59° C., about 59.5° C., about 60° C., about 60.5° C., about 61° C., about 61.5° C., about 62° C., about 62.5° C., about 63° C., about 63.5° C., about 64° C., about 64.5° C., about 65° C., about 65.5° C., about 66° C., about 66.5° C., about 67° C., about 67.5° C., about 68° C., about 68.5° C., about 69° C., about 69.5° C., about 70° C., about 70.5° C., or about 71° C. In other embodiments, the second temperature is selected from the range of ±5° C., ±4° C., ±3° C., ±2° C., or ±1° C. of the polymerase's optimal elongation temperature.

In various embodiments, the first temperature is about 1° C. to about 30° C. higher or lower than the second temperature. In various embodiments, the first temperature is about 1° C. to about 25° C. higher or lower than the second temperature. In various embodiments, the first temperature is about 1° C. to about 20° C. higher or lower than the second temperature. In some embodiments, the first temperature is about 1° C. higher or lower than the second temperature. In some embodiments, the first temperature is about 1.5° C. higher or lower than the second temperature. In some embodiments, the first temperature is about 2° C. higher or lower than the second temperature. In some embodiments, the first temperature is about 2.5° C. higher or lower than the second temperature. In some embodiments, the first temperature is about 3° C. higher or lower than the second temperature. In some embodiments, the first temperature is about 3.5° C. higher or lower than the second temperature. In some embodiments, the first temperature is about 4° C. higher or lower than the second temperature. In some embodiments, the first temperature is about 4.5° C. higher or lower than the second temperature. In some embodiments, the first temperature is about 5° C. higher or lower than the second temperature. In some embodiments, the first temperature is about 5.5° C. higher or lower than the second temperature. In some embodiments, the first temperature is about 6° C. higher or lower than the second temperature. In some embodiments, the first temperature is about 6.5° C. higher or lower than the second temperature. In some embodiments, the first temperature is about 7° C. higher or lower than the second temperature. In some embodiments, the first temperature is about 7.5° C. higher or lower than the second temperature. In some embodiments, the first temperature is about 8° C. higher or lower than the second temperature. In some embodiments, the first temperature is about 8.5° C. higher or lower than the second temperature. In some embodiments, the first temperature is about 9° C. higher or lower than the second temperature. In some embodiments, the first temperature is about 9.5° C. higher or lower than the second temperature. In some embodiments, the first temperature is about 10° C. higher or lower than the second temperature. In some embodiments, the first temperature is about 10.5° C. higher or lower than the second temperature. In some embodiments, the first temperature is about 11° C. higher or lower than the second temperature. In some embodiments, the first temperature is about 11.5° C. higher or lower than the second temperature. In some embodiments, the first temperature is about 12° C. higher or lower than the second temperature. In some embodiments, the first temperature is about 12.5° C. higher or lower than the second temperature. In some embodiments, the first temperature is about 13° C. higher or lower than the second temperature. In some embodiments, the first temperature is about 13.5° C. higher or lower than the second temperature. In some embodiments, the first temperature is about 14° C. higher or lower than the second temperature. In some embodiments, the first temperature is about 14.5° C. higher or lower than the second temperature. In some embodiments, the first temperature is about 15° C. higher or lower than the second temperature. In some embodiments, the first temperature is about 15.5° C. higher or lower than the second temperature. In some embodiments, the first temperature is about 16° C. higher or lower than the second temperature. In some embodiments, the first temperature is about 16.5° C. higher or lower than the second temperature. In some embodiments, the first temperature is about 17° C. higher or lower than the second temperature. In some embodiments, the first temperature is about 17.5° C. higher or lower than the second temperature. In some embodiments, the first temperature is about 18° C. higher or lower than the second temperature. In some embodiments, the first temperature is about 18.5° C. higher or lower than the second temperature. In some embodiments, the first temperature is about 19° C. higher or lower than the second temperature. In some embodiments, the first temperature is about 19.5° C. higher or lower than the second temperature. In some embodiments, the first temperature is about 20° C. higher or lower than the second temperature. In some embodiments, the first temperature is about 20.5° C. higher or lower than the second temperature. In some embodiments, the first temperature is about 21° C. higher or lower than the second temperature. In some embodiments, the first temperature is about 21.5° C. higher or lower than the second temperature. In some embodiments, the first temperature is about 22° C. higher or lower than the second temperature. In some embodiments, the first temperature is about 22.5° C. higher or lower than the second temperature. In some embodiments, the first temperature is about 23° C. higher or lower than the second temperature. In some embodiments, the first temperature is about 23.5° C. higher or lower than the second temperature. In some embodiments, the first temperature is about 24° C. higher or lower than the second temperature. In some embodiments, the first temperature is about 24.5° C. higher or lower than the second temperature. In some embodiments, the first temperature is about 25° C. higher or lower than the second temperature. In some embodiments, the first temperature is about 25.5° C. higher or lower than the second temperature. In some embodiments, the first temperature is about 26° C. higher or lower than the second temperature. In some embodiments, the first temperature is about 26.5° C. higher or lower than the second temperature. In some embodiments, the first temperature is about 27° C. higher or lower than the second temperature. In some embodiments, the first temperature is about 27.5° C. higher or lower than the second temperature. In some embodiments, the first temperature is about 28° C. higher or lower than the second temperature. In some embodiments, the first temperature is about 28.5° C. higher or lower than the second temperature. In some embodiments, the first temperature is about 29° C. higher or lower than the second temperature. In some embodiments, the first temperature is about 29.5° C. higher or lower than the second temperature. In some embodiments, the first temperature is about 30° C. higher or lower than the second temperature.

In some embodiments, the first temperature is not more than about 25° C. higher than the second temperature, and the first temperature is equal to or less than about 90° C. In some embodiments, the first temperature is not more than about 25° C. higher than the second temperature, and the first temperature is equal to or less than about 89° C. In some embodiments, the first temperature is not more than about 25° C. higher than the second temperature, and the first temperature is equal to or less than about 88° C. In some embodiments, the first temperature is not more than about 25° C. higher than the second temperature, and the first temperature is equal to or less than about 87° C. In some embodiments, the first temperature is not more than about 25° C. higher than the second temperature, and the first temperature is equal to or less than about 86° C. In some embodiments, the first temperature is not more than about 25° C. higher than the second temperature, and the first temperature is equal to or less than about 85° C. In any embodiments described in this paragraph, the polymerase can be a Bst DNA polymerase, a Taq DNA polymerase, a DNA polymerase I, a Vent® DNA polymerase, a phi29 DNA polymerase, or a truncated or mutated version of any of these polymerases.

In some embodiments, the polymerase is a Bst DNA polymerase, and the first temperature is selected from the range of about 68-78° C., and the second temperature is selected from the range of about 55-69° C. Specifically, in particular embodiments where the polymerase is a Bst DNA polymerase, the first temperature is about 68° C., and the second temperature is selected from the range of about 55-69° C. In particular embodiments where the polymerase is a Bst DNA polymerase, the first temperature is about 68.5° C., and the second temperature is selected from the range of about 55-69° C. In particular embodiments where the polymerase is a Bst DNA polymerase, the first temperature is about 69° C., and the second temperature is selected from the range of about 55-69° C. In particular embodiments where the polymerase is a Bst DNA polymerase, the first temperature is about 69.5° C., and the second temperature is selected from the range of about 55-69° C. In particular embodiments where the polymerase is a Bst DNA polymerase, the first temperature is about 69° C., and the second temperature is selected from the range of about 55-69° C. In particular embodiments where the polymerase is a Bst DNA polymerase, the first temperature is about 69.5° C., and the second temperature is selected from the range of about 55-69° C. In particular embodiments where the polymerase is a Bst DNA polymerase, the first temperature is about 70° C., and the second temperature is selected from the range of about 55-69° C. In particular embodiments where the polymerase is a Bst DNA polymerase, the first temperature is about 70.5° C., and the second temperature is selected from the range of about 55-69° C. In particular embodiments where the polymerase is a Bst DNA polymerase, the first temperature is about 71° C., and the second temperature is selected from the range of about 55-69° C. In particular embodiments where the polymerase is a Bst DNA polymerase, the first temperature is about 71.5° C., and the second temperature is selected from the range of about 55-69° C. In particular embodiments where the polymerase is a Bst DNA polymerase, the first temperature is about 72° C., and the second temperature is selected from the range of about 55-69° C. In particular embodiments where the polymerase is a Bst DNA polymerase, the first temperature is about 72.5° C., and the second temperature is selected from the range of about 55-69° C. In particular embodiments where the polymerase is a Bst DNA polymerase, the first temperature is about 73° C., and the second temperature is selected from the range of about 55-69° C. In particular embodiments where the polymerase is a Bst DNA polymerase, the first temperature is about 73.5° C., and the second temperature is selected from the range of about 55-69° C. In particular embodiments where the polymerase is a Bst DNA polymerase, the first temperature is about 74° C., and the second temperature is selected from the range of about 55-69° C. In particular embodiments where the polymerase is a Bst DNA polymerase, the first temperature is about 74.5° C., and the second temperature is selected from the range of about 55-69° C. In particular embodiments where the polymerase is a Bst DNA polymerase, the first temperature is about 75° C., and the second temperature is selected from the range of about 55-69° C. In particular embodiments where the polymerase is a Bst DNA polymerase, the first temperature is about 75.5° C., and the second temperature is selected from the range of about 55-69° C. In particular embodiments where the polymerase is a Bst DNA polymerase, the first temperature is about 76° C., and the second temperature is selected from the range of about 55-69° C. In particular embodiments where the polymerase is a Bst DNA polymerase, the first temperature is about 76.5° C., and the second temperature is selected from the range of about 55-69° C. In particular embodiments where the polymerase is a Bst DNA polymerase, the first temperature is about 77° C., and the second temperature is selected from the range of about 55-69° C. In particular embodiments where the polymerase is a Bst DNA polymerase, the first temperature is about 77.5° C., and the second temperature is selected from the range of about 55-69° C. In particular embodiments where the polymerase is a Bst DNA polymerase, the first temperature is about 78° C., and the second temperature is selected from the range of about 55-69° C. Particularly, in any of the embodiments described in this paragraph, the second temperature selected from the range of about 55-69° C. can be about 55° C., 55.5° C., 56° C., 56.5° C., 57° C., 57.5° C., 58° C., 58.5° C., 59° C., 59.5° C., 60° C., 60.5° C., 61° C., 61.5° C., 62° C., 62.5° C., 63° C., 63.5° C., 64° C., 64.5° C., 65° C., 65.5° C., 66° C., 66.5° C., 67° C., 67.5° C., 68° C., 68.5° C., or 69° C. Particularly, in any of the embodiments described in this paragraph, the Bst DNA polymerase can be either the wild-type Bst DNA polymerase, or a mutated or truncated bst DNA polymerase selected from Bst DNA Polymerase, Large Fragment, Bst 2.0 DNA Polymerase, Bst 2.0 WarmStart DNA Polymerase and Bst 3.0 DNA Polymerase.

In some embodiments, the polymerase is a Bst DNA polymerase, and the first temperature is selected from the range of about 68-78° C., and the second temperature is selected from the range of about 55-69° C. Specifically, in particular embodiments where the polymerase is a Bst DNA polymerase, the first temperature is selected from the range of about 68-78° C., and the second temperature is about 55° C. In particular embodiments where the polymerase is a Bst DNA polymerase, the first temperature is selected from the range of about 68-78° C., and the second temperature is about 55.5° C. In particular embodiments where the polymerase is a Bst DNA polymerase, the first temperature is selected from the range of about 68-78° C., and the second temperature is about 56° C. In particular embodiments where the polymerase is a Bst DNA polymerase, the first temperature is selected from the range of about 68-78° C., and the second temperature is about 56.5° C. In particular embodiments where the polymerase is a Bst DNA polymerase, the first temperature is selected from the range of about 68-78° C., and the second temperature is about 57° C. In particular embodiments where the polymerase is a Bst DNA polymerase, the first temperature is selected from the range of about 68-78° C., and the second temperature is about 57.5° C. In particular embodiments where the polymerase is a Bst DNA polymerase, the first temperature is selected from the range of about 68-78° C., and the second temperature is about 58° C. In particular embodiments where the polymerase is a Bst DNA polymerase, the first temperature is selected from the range of about 68-78° C., and the second temperature is about 58.5° C. In particular embodiments where the polymerase is a Bst DNA polymerase, the first temperature is selected from the range of about 68-78° C., and the second temperature is about 59° C. In particular embodiments where the polymerase is a Bst DNA polymerase, the first temperature is selected from the range of about 68-78° C., and the second temperature is about 59.5° C. In particular embodiments where the polymerase is a Bst DNA polymerase, the first temperature is selected from the range of about 68-78° C., and the second temperature is about 60° C. In particular embodiments where the polymerase is a Bst DNA polymerase, the first temperature is selected from the range of about 68-78° C., and the second temperature is about 60.5° C. In particular embodiments where the polymerase is a Bst DNA polymerase, the first temperature is selected from the range of about 68-78° C., and the second temperature is about 61° C. In particular embodiments where the polymerase is a Bst DNA polymerase, the first temperature is selected from the range of about 68-78° C., and the second temperature is about 61.5° C. In particular embodiments where the polymerase is a Bst DNA polymerase, the first temperature is selected from the range of about 68-78° C., and the second temperature is about 62° C. In particular embodiments where the polymerase is a Bst DNA polymerase, the first temperature is selected from the range of about 68-78° C., and the second temperature is about 62.5° C. In particular embodiments where the polymerase is a Bst DNA polymerase, the first temperature is selected from the range of about 68-78° C., and the second temperature is about 63° C. In particular embodiments where the polymerase is a Bst DNA polymerase, the first temperature is selected from the range of about 68-78° C., and the second temperature is about 63.5° C. In particular embodiments where the polymerase is a Bst DNA polymerase, the first temperature is selected from the range of about 68-78° C., and the second temperature is about 64° C. In particular embodiments where the polymerase is a Bst DNA polymerase, the first temperature is selected from the range of about 68-78° C., and the second temperature is about 64.5° C. In particular embodiments where the polymerase is a Bst DNA polymerase, the first temperature is selected from the range of about 68-78° C., and the second temperature is about 65° C. In particular embodiments where the polymerase is a Bst DNA polymerase, the first temperature is selected from the range of about 68-78° C., and the second temperature is about 65.5° C. In particular embodiments where the polymerase is a Bst DNA polymerase, the first temperature is selected from the range of about 68-78° C., and the second temperature is about 66° C. In particular embodiments where the polymerase is a Bst DNA polymerase, the first temperature is selected from the range of about 68-78° C., and the second temperature is about 66.5° C. In particular embodiments where the polymerase is a Bst DNA polymerase, the first temperature is selected from the range of about 68-78° C., and the second temperature is about 67° C. In particular embodiments where the polymerase is a Bst DNA polymerase, the first temperature is selected from the range of about 68-78° C., and the second temperature is about 67.5° C. In particular embodiments where the polymerase is a Bst DNA polymerase, the first temperature is selected from the range of about 68-78° C., and the second temperature is about 68° C. In particular embodiments where the polymerase is a Bst DNA polymerase, the first temperature is selected from the range of about 68-78° C., and the second temperature is about 68.5° C. In particular embodiments where the polymerase is a Bst DNA polymerase, the first temperature is selected from the range of about 68-78° C., and the second temperature is about 69° C. Particularly, in any of the embodiments described in this paragraph, the first temperature selected from the range of about 68-78° C. can be about 68° C., 68.5° C., 69° C., 69.5° C., 70° C., 70.5° C., 71° C., 71.5° C., 72° C., 72.5° C., 73° C., 73.5° C., 74° C., 74.5° C., 75° C., 75.5° C., 76° C., 76.5° C., 77° C., 77.5° C., or 78° C. Particularly, in any of the embodiments described in this paragraph, the Bst DNA polymerase can be either the wild-type Bst DNA polymerase, or a mutated or truncated bst DNA polymerase selected from Bst DNA Polymerase, Large Fragment, Bst 2.0 DNA Polymerase, Bst 2.0 WarmStart DNA Polymerase and Bst 3.0 DNA Polymerase.

In some embodiments, the polymerase is a Bst DNA polymerase, and the first temperature is selected from the range of about 72-76° C., and the second temperature is selected from the range of about 61-65° C. Specifically, in particular embodiments where the polymerase is a Bst DNA polymerase, the first temperature is about 72° C., and the second temperature is about 61° C., about 62° C., about 63° C., about 64° C. or about 65° C. In particular embodiments where the polymerase is a Bst DNA polymerase, the first temperature is about 73° C., and the second temperature is about 61° C., about 62° C., about 63° C., about 64° C. or about 65° C. In particular embodiments where the polymerase is a Bst DNA polymerase, the first temperature is about 74° C., and the second temperature is about 61° C., about 62° C., about 63° C., about 64° C. or about 65° C. In particular embodiments where the polymerase is a Bst DNA polymerase, the first temperature is about 75° C., and the second temperature is about 61° C., about 62° C., about 63° C., about 64° C. or about 65° C. In particular embodiments where the polymerase is a Bst DNA polymerase, the first temperature is about 76° C., and the second temperature is about 61° C., about 62° C., about 63° C., about 64° C. or about 65° C. In particular embodiments where the polymerase is a Bst DNA polymerase, the first temperature is about 76° C., and the second temperature is about 62° C. In particular embodiments where the polymerase is a Bst DNA polymerase, the first temperature is about 76° C., and the second temperature is about 61° C. Particularly, in any of the embodiments described in this paragraph, the Bst DNA polymerase can be either the wild-type Bst DNA polymerase, or a mutated or truncated Bst DNA polymerase selected from Bst DNA Polymerase, Large Fragment, Bst 2.0 DNA Polymerase, Bst 2.0 WarmStart DNA Polymerase and Bst 3.0 DNA Polymerase.

In some embodiments, the polymerase is a Taq DNA polymerase, or a truncated or mutated version thereof, and the first temperature is selected from the range of about 70-88° C., and the second temperature is selected from the range of about 58-70° C. Specifically, in particular embodiments where the polymerase is a Taq DNA polymerase, the first temperature is about 70° C., and the second temperature is selected from the range of about 58-70° C. In particular embodiments where the polymerase is a Taq DNA polymerase, the first temperature is about 70.5° C., and the second temperature is selected from the range of about 58-70° C. In particular embodiments where the polymerase is a Taq DNA polymerase, the first temperature is about 71° C., and the second temperature is selected from the range of about 58-70° C. In particular embodiments where the polymerase is a Taq DNA polymerase, the first temperature is about 71.5° C., and the second temperature is selected from the range of about 58-70° C. In particular embodiments where the polymerase is a Taq DNA polymerase, the first temperature is about 72° C., and the second temperature is selected from the range of about 58-70° C. In particular embodiments where the polymerase is a Taq DNA polymerase, the first temperature is about 72.5° C., and the second temperature is selected from the range of about 58-70° C. In particular embodiments where the polymerase is a Taq DNA polymerase, the first temperature is about 73° C., and the second temperature is selected from the range of about 58-70° C. In particular embodiments where the polymerase is a Taq DNA polymerase, the first temperature is about 73.5° C., and the second temperature is selected from the range of about 58-70° C. In particular embodiments where the polymerase is a Taq DNA polymerase, the first temperature is about 74° C., and the second temperature is selected from the range of about 58-70° C. In particular embodiments where the polymerase is a Taq DNA polymerase, the first temperature is about 74.5° C., and the second temperature is selected from the range of about 58-70° C. In particular embodiments where the polymerase is a Taq DNA polymerase, the first temperature is about 75° C., and the second temperature is selected from the range of about 58-70° C. In particular embodiments where the polymerase is a Taq DNA polymerase, the first temperature is about 75.5° C., and the second temperature is selected from the range of about 58-70° C. In particular embodiments where the polymerase is a Taq DNA polymerase, the first temperature is about 76° C., and the second temperature is selected from the range of about 58-70° C. In particular embodiments where the polymerase is a Taq DNA polymerase, the first temperature is about 76.5° C., and the second temperature is selected from the range of about 58-70° C. In particular embodiments where the polymerase is a Taq DNA polymerase, the first temperature is about 77° C., and the second temperature is selected from the range of about 58-70° C. In particular embodiments where the polymerase is a Taq DNA polymerase, the first temperature is about 77.5° C., and the second temperature is selected from the range of about 58-70° C. In particular embodiments where the polymerase is a Taq DNA polymerase, the first temperature is about 78° C., and the second temperature is selected from the range of about 58-70° C. In particular embodiments where the polymerase is a Taq DNA polymerase, the first temperature is about 78.5° C., and the second temperature is selected from the range of about 58-70° C. In particular embodiments where the polymerase is a Taq DNA polymerase, the first temperature is about 79° C., and the second temperature is selected from the range of about 58-70° C. In particular embodiments where the polymerase is a Taq DNA polymerase, the first temperature is about 79.5° C., and the second temperature is selected from the range of about 58-70° C. In particular embodiments where the polymerase is a Taq DNA polymerase, the first temperature is about 80° C., and the second temperature is selected from the range of about 58-70° C. In particular embodiments where the polymerase is a Taq DNA polymerase, the first temperature is about 81.5° C., and the second temperature is selected from the range of about 58-70° C. In particular embodiments where the polymerase is a Taq DNA polymerase, the first temperature is about 82° C., and the second temperature is selected from the range of about 58-70° C. In particular embodiments where the polymerase is a Taq DNA polymerase, the first temperature is about 82.5° C., and the second temperature is selected from the range of about 58-70° C. In particular embodiments where the polymerase is a Taq DNA polymerase, the first temperature is about 83° C., and the second temperature is selected from the range of about 58-70° C. In particular embodiments where the polymerase is a Taq DNA polymerase, the first temperature is about 83.5° C., and the second temperature is selected from the range of about 58-70° C. In particular embodiments where the polymerase is a Taq DNA polymerase, the first temperature is about 84° C., and the second temperature is selected from the range of about 58-70° C. In particular embodiments where the polymerase is a Taq DNA polymerase, the first temperature is about 84.5° C., and the second temperature is selected from the range of about 58-70° C. In particular embodiments where the polymerase is a Taq DNA polymerase, the first temperature is about 85° C., and the second temperature is selected from the range of about 58-70° C. In particular embodiments where the polymerase is a Taq DNA polymerase, the first temperature is about 85.5° C., and the second temperature is selected from the range of about 58-70° C. In particular embodiments where the polymerase is a Taq DNA polymerase, the first temperature is about 86° C., and the second temperature is selected from the range of about 58-70° C. In particular embodiments where the polymerase is a Taq DNA polymerase, the first temperature is about 86.5° C., and the second temperature is selected from the range of about 58-70° C. In particular embodiments where the polymerase is a Taq DNA polymerase, the first temperature is about 87° C., and the second temperature is selected from the range of about 58-70° C. In particular embodiments where the polymerase is a Taq DNA polymerase, the first temperature is about 87.5° C., and the second temperature is selected from the range of about 58-70° C. In particular embodiments where the polymerase is a Taq DNA polymerase, the first temperature is about 88° C., and the second temperature is selected from the range of about 58-70° C. Particularly, in any of the embodiments described in this paragraph, the second temperature selected from the range of about 58-70° C. can be about 58° C., 58.5° C., 59° C., 59.5° C., 60° C., 60.5° C., 61° C., 61.5° C., 62° C., 62.5° C., 63° C., 63.5° C., 64° C., 64.5° C., 65° C., 65.5° C., 66° C., 66.5° C., 67° C., 67.5° C., 68° C., 68.5° C., 69° C., 69.5° C., or 70° C. Particularly, in any of the embodiments described in this paragraph, the Taq DNA polymerase can be either the wild-type Taq DNA polymerase, or a mutated or truncated Taq DNA polymerase selected from Hot Start Taq DNA Polymerase, EpiMark Hot Start Taq DNA Polymerase, OneTaq DNA Polymerase, OneTaq Hot Start DNA Polymerase, LongAmp Taq DNA Polymerase, or LongTaq DNA Polymerase.

In some embodiments, the polymerase is a Taq DNA polymerase, or a truncated or mutated version thereof, and the first temperature is selected from the range of about 70-88° C., and the second temperature is selected from the range of about 58-70° C. Specifically, in particular embodiments where the polymerase is a Taq DNA polymerase, the first temperature is selected from the range of about 70-88° C., and the second temperature is about 58° C. In particular embodiments where the polymerase is a Taq DNA polymerase, the first temperature is selected from the range of about 70-88° C., and the second temperature is about 58.5° C. In particular embodiments where the polymerase is a Taq DNA polymerase, the first temperature is selected from the range of about 70-88° C., and the second temperature is about 59° C. In particular embodiments where the polymerase is a Taq DNA polymerase, the first temperature is selected from the range of about 70-88° C., and the second temperature is about 59.5° C. In particular embodiments where the polymerase is a Taq DNA polymerase, the first temperature is selected from the range of about 70-88° C., and the second temperature is about 60° C. In particular embodiments where the polymerase is a Taq DNA polymerase, the first temperature is selected from the range of about 70-88° C., and the second temperature is about 60.5° C. In particular embodiments where the polymerase is a Taq DNA polymerase, the first temperature is selected from the range of about 70-88° C., and the second temperature is about 61° C. In particular embodiments where the polymerase is a Taq DNA polymerase, the first temperature is selected from the range of about 70-88° C., and the second temperature is about 61.5° C. In particular embodiments where the polymerase is a Taq DNA polymerase, the first temperature is selected from the range of about 70-88° C., and the second temperature is about 62° C. In particular embodiments where the polymerase is a Taq DNA polymerase, the first temperature is selected from the range of about 70-88° C., and the second temperature is about 62.5° C. In particular embodiments where the polymerase is a Taq DNA polymerase, the first temperature is selected from the range of about 70-88° C., and the second temperature is about 63° C. In particular embodiments where the polymerase is a Taq DNA polymerase, the first temperature is selected from the range of about 70-88° C., and the second temperature is about 63.5° C. In particular embodiments where the polymerase is a Taq DNA polymerase, the first temperature is selected from the range of about 70-88° C., and the second temperature is about 64° C. In particular embodiments where the polymerase is a Taq DNA polymerase, the first temperature is selected from the range of about 70-88° C., and the second temperature is about 64.5° C. In particular embodiments where the polymerase is a Taq DNA polymerase, the first temperature is selected from the range of about 70-88° C., and the second temperature is about 65° C. In particular embodiments where the polymerase is a Taq DNA polymerase, the first temperature is selected from the range of about 70-88° C., and the second temperature is about 65.5° C. In particular embodiments where the polymerase is a Taq DNA polymerase, the first temperature is selected from the range of about 70-88° C., and the second temperature is about 66° C. In particular embodiments where the polymerase is a Taq DNA polymerase, the first temperature is selected from the range of about 70-88° C., and the second temperature is about 66.5° C. In particular embodiments where the polymerase is a Taq DNA polymerase, the first temperature is selected from the range of about 70-88° C., and the second temperature is about 67° C. In particular embodiments where the polymerase is a Taq DNA polymerase, the first temperature is selected from the range of about 70-88° C., and the second temperature is about 67.5° C. In particular embodiments where the polymerase is a Taq DNA polymerase, the first temperature is selected from the range of about 70-88° C., and the second temperature is about 68° C. In particular embodiments where the polymerase is a Taq DNA polymerase, the first temperature is selected from the range of about 70-88° C., and the second temperature is about 68.5° C. In particular embodiments where the polymerase is a Taq DNA polymerase, the first temperature is selected from the range of about 70-88° C., and the second temperature is about 69° C. In particular embodiments where the polymerase is a Taq DNA polymerase, the first temperature is selected from the range of about 70-88° C., and the second temperature is about 69.5° C. In particular embodiments where the polymerase is a Taq DNA polymerase, the first temperature is selected from the range of about 70-88° C., and the second temperature is about 70° C. Particularly, in any of the embodiments described in this paragraph, the first temperature selected from the range of about 70-88° C. can be about 70° C., 70.5° C., 71° C., 71.5° C., 72° C., 72.5° C., 73° C., 73.5° C., 74° C., 74.5° C., 75° C., 75.5° C., 76° C., 76.5° C., 77° C., 77.5° C., 78° C., 78.5° C., 79° C., 79.5° C., 80° C., 80.5° C., 81° C., 81.5° C., 82° C., 82.5° C., 83° C., 83.5° C., 84° C., 84.5° C., 85° C., 85.5° C., 86° C., 86.5° C., 87° C., 87.5° C., or 88° C. Particularly, in any of the embodiments described in this paragraph, the Taq DNA polymerase can be either the wild-type Taq DNA polymerase, or a mutated or truncated Taq DNA polymerase selected from Hot Start Taq DNA Polymerase, EpiMark Hot Start Taq DNA Polymerase, OneTaq DNA Polymerase, OneTaq Hot Start DNA Polymerase, LongAmp Taq DNA Polymerase, or LongTaq DNA Polymerase.

In some embodiments, the polymerase is a DNA polymerase I or a truncated or mutated version thereof, and the first temperature is selected from the range of about 50-60° C., and the second temperature is selected from the range of about 30-40° C. Specifically, in particular embodiments where the polymerase is a DNA polymerase I, the first temperature is about 50° C., and the second temperature is selected from the range of about 30-40° C. In particular embodiments where the polymerase is a DNA polymerase I, the first temperature is about 50.5° C., and the second temperature is selected from the range of about 30-40° C. In particular embodiments where the polymerase is a DNA polymerase I, the first temperature is about 51° C., and the second temperature is selected from the range of about 30-40° C. In particular embodiments where the polymerase is a DNA polymerase I, the first temperature is about 51.5° C., and the second temperature is selected from the range of about 30-40° C. In particular embodiments where the polymerase is a DNA polymerase I, the first temperature is about 52° C., and the second temperature is selected from the range of about 30-40° C. In particular embodiments where the polymerase is a DNA polymerase I, the first temperature is about 52.5° C., and the second temperature is selected from the range of about 30-40° C. In particular embodiments where the polymerase is a DNA polymerase I, the first temperature is about 53° C., and the second temperature is selected from the range of about 30-40° C. In particular embodiments where the polymerase is a DNA polymerase I, the first temperature is about 53.5° C., and the second temperature is selected from the range of about 30-40° C. In particular embodiments where the polymerase is a DNA polymerase I, the first temperature is about 54° C., and the second temperature is selected from the range of about 30-40° C. In particular embodiments where the polymerase is a DNA polymerase I, the first temperature is about 54.5° C., and the second temperature is selected from the range of about 30-40° C. In particular embodiments where the polymerase is a DNA polymerase I, the first temperature is about 55° C., and the second temperature is selected from the range of about 30-40° C. In particular embodiments where the polymerase is a DNA polymerase I, the first temperature is about 55.5° C., and the second temperature is selected from the range of about 30-40° C. In particular embodiments where the polymerase is a DNA polymerase I, the first temperature is about 56° C., and the second temperature is selected from the range of about 30-40° C. In particular embodiments where the polymerase is a DNA polymerase I, the first temperature is about 56.5° C., and the second temperature is selected from the range of about 30-40° C. In particular embodiments where the polymerase is a DNA polymerase I, the first temperature is about 57° C., and the second temperature is selected from the range of about 30-40° C. In particular embodiments where the polymerase is a DNA polymerase I, the first temperature is about 57.5° C., and the second temperature is selected from the range of about 30-40° C. In particular embodiments where the polymerase is a DNA polymerase I, the first temperature is about 58° C., and the second temperature is selected from the range of about 30-40° C. In particular embodiments where the polymerase is a DNA polymerase I, the first temperature is about 58.5° C., and the second temperature is selected from the range of about 30-40° C. In particular embodiments where the polymerase is a DNA polymerase I, the first temperature is about 59° C., and the second temperature is selected from the range of about 30-40° C. In particular embodiments where the polymerase is a DNA polymerase I, the first temperature is about 59.5° C., and the second temperature is selected from the range of about 30-40° C. In particular embodiments where the polymerase is a DNA polymerase I, the first temperature is about 60° C., and the second temperature is selected from the range of about 30-40° C. Particularly, in any of the embodiments described in this paragraph, the second temperature is selected from the range of about 30-40° C. can be about 30° C., 30.5° C., 31° C., 31.5° C., 32° C., 32.5° C., 33° C., 33.5° C., 34° C., 34.5° C., 35° C., 35.5° C., 36° C., 36.5° C., 37° C., 37.5° C., 38° C., 38.5° C., 39° C., 39.5° C., or 40° C. Particularly, in any of the embodiments described in this paragraph, the polymerase can be selected from the wild-type DNA polymerase I, DNA polymerase I, large (Klenow) fragment, or Klenow exo⁻.

In some embodiments, the polymerase is a DNA polymerase I or a truncated or mutated version thereof, and the first temperature is selected from the range of about 50-60° C., and the second temperature is selected from the range of about 30-40° C. Specifically, in particular embodiments where the polymerase is a DNA polymerase I, the first temperature is selected from the range of about 50-60° C., and the second temperature is about 30° C. In particular embodiments where the polymerase is a DNA polymerase I, the first temperature is selected from the range of about 50-60° C., and the second temperature is about 30.5° C. In particular embodiments where the polymerase is a DNA polymerase I, the first temperature is selected from the range of about 50-60° C., and the second temperature is about 31° C. In particular embodiments where the polymerase is a DNA polymerase I, the first temperature is selected from the range of about 50-60° C., and the second temperature is about 31.5° C. In particular embodiments where the polymerase is a DNA polymerase I, the first temperature is selected from the range of about 50-60° C., and the second temperature is about 32° C. In particular embodiments where the polymerase is a DNA polymerase I, the first temperature is selected from the range of about 50-60° C., and the second temperature is about 32.5° C. In particular embodiments where the polymerase is a DNA polymerase I, the first temperature is selected from the range of about 50-60° C., and the second temperature is about 33° C. In particular embodiments where the polymerase is a DNA polymerase I, the first temperature is selected from the range of about 50-60° C., and the second temperature is about 33.5° C. In particular embodiments where the polymerase is a DNA polymerase I, the first temperature is selected from the range of about 50-60° C., and the second temperature is about 34° C. In particular embodiments where the polymerase is a DNA polymerase I, the first temperature is selected from the range of about 50-60° C., and the second temperature is about 34.5° C. In particular embodiments where the polymerase is a DNA polymerase I, the first temperature is selected from the range of about 50-60° C., and the second temperature is about 35° C. In particular embodiments where the polymerase is a DNA polymerase I, the first temperature is selected from the range of about 50-60° C., and the second temperature is about 35.5° C. In particular embodiments where the polymerase is a DNA polymerase I, the first temperature is selected from the range of about 50-60° C., and the second temperature is about 36° C. In particular embodiments where the polymerase is a DNA polymerase I, the first temperature is selected from the range of about 50-60° C., and the second temperature is about 36.5° C. In particular embodiments where the polymerase is a DNA polymerase I, the first temperature is selected from the range of about 50-60° C., and the second temperature is about 37° C. In particular embodiments where the polymerase is a DNA polymerase I, the first temperature is selected from the range of about 50-60° C., and the second temperature is about 37.5° C. In particular embodiments where the polymerase is a DNA polymerase I, the first temperature is selected from the range of about 50-60° C., and the second temperature is about 38° C. In particular embodiments where the polymerase is a DNA polymerase I, the first temperature is selected from the range of about 50-60° C., and the second temperature is about 38.5° C. In particular embodiments where the polymerase is a DNA polymerase I, the first temperature is selected from the range of about 50-60° C., and the second temperature is about 39° C. In particular embodiments where the polymerase is a DNA polymerase I, the first temperature is selected from the range of about 50-60° C., and the second temperature is about 39.5° C. In particular embodiments where the polymerase is a DNA polymerase I, the first temperature is selected from the range of about 50-60° C., and the second temperature is about 40° C. Particularly, in any of the embodiments described in this paragraph, the second temperature is selected from the range of about 50-60° C. can be about 50° C., 50.5° C., 51° C., 51.5° C., 52° C., 52.5° C., 53° C., 53.5° C., 54° C., 54.5° C., 55° C., 55.5° C., 56° C., 56.5° C., 57° C., 57.5° C., 58° C., 58.5° C., 59° C., 59.5° C., or 60° C. Particularly, in any of the embodiments described in this paragraph, the polymerase can be selected from the wild-type DNA polymerase I, DNA polymerase I, large (Klenow) fragment, or Klenow exo⁻.

In some embodiments, the polymerase is a Vent® DNA polymerase or a truncated or mutated version thereof, and the first temperature is selected from the range of about 70-80° C., and the second temperature is selected from the range of about 55-70° C. Specifically, in particular embodiments where the polymerase is a Vent® DNA polymerase, the first temperature is about 70° C., and the second temperature is selected from the range of about 55-70° C. In particular embodiments where the polymerase is a Vent® DNA polymerase, the first temperature is about 70.5° C., and the second temperature is selected from the range of about 55-70° C. In particular embodiments where the polymerase is a Vent® DNA polymerase, the first temperature is about 71° C., and the second temperature is selected from the range of about 55-70° C. In particular embodiments where the polymerase is a Vent® DNA polymerase, the first temperature is about 71.5° C., and the second temperature is selected from the range of about 55-70° C. In particular embodiments where the polymerase is a Vent® DNA polymerase, the first temperature is about 72° C., and the second temperature is selected from the range of about 55-70° C. In particular embodiments where the polymerase is a Vent® DNA polymerase, the first temperature is about 72.5° C., and the second temperature is selected from the range of about 55-70° C. In particular embodiments where the polymerase is a Vent® DNA polymerase, the first temperature is about 73° C., and the second temperature is selected from the range of about 55-70° C. In particular embodiments where the polymerase is a Vent® DNA polymerase, the first temperature is about 73.5° C., and the second temperature is selected from the range of about 55-70° C. In particular embodiments where the polymerase is a Vent® DNA polymerase, the first temperature is about 74° C., and the second temperature is selected from the range of about 55-70° C. In particular embodiments where the polymerase is a Vent® DNA polymerase, the first temperature is about 74.5° C., and the second temperature is selected from the range of about 55-70° C. In particular embodiments where the polymerase is a Vent® DNA polymerase, the first temperature is about 75° C., and the second temperature is selected from the range of about 55-70° C. In particular embodiments where the polymerase is a Vent® DNA polymerase, the first temperature is about 75.5° C., and the second temperature is selected from the range of about 55-70° C. In particular embodiments where the polymerase is a Vent® DNA polymerase, the first temperature is about 76° C., and the second temperature is selected from the range of about 55-70° C. In particular embodiments where the polymerase is a Vent® DNA polymerase, the first temperature is about 76.5° C., and the second temperature is selected from the range of about 55-70° C. In particular embodiments where the polymerase is a Vent® DNA polymerase, the first temperature is about 77° C., and the second temperature is selected from the range of about 55-70° C. In particular embodiments where the polymerase is a Vent® DNA polymerase, the first temperature is about 77.5° C., and the second temperature is selected from the range of about 55-70° C. In particular embodiments where the polymerase is a Vent® DNA polymerase, the first temperature is about 78° C., and the second temperature is selected from the range of about 55-70° C. In particular embodiments where the polymerase is a Vent® DNA polymerase, the first temperature is about 78.5° C., and the second temperature is selected from the range of about 55-70° C. In particular embodiments where the polymerase is a Vent® DNA polymerase, the first temperature is about 79° C., and the second temperature is selected from the range of about 55-70° C. In particular embodiments where the polymerase is a Vent® DNA polymerase, the first temperature is about 79.5° C., and the second temperature is selected from the range of about 55-70° C. In particular embodiments where the polymerase is a Vent® DNA polymerase, the first temperature is about 80° C., and the second temperature is selected from the range of about 55-70° C. Particularly, in any of the embodiments described in this paragraph, the second temperature selected from the range of about 55-70° C. can be about 55° C., 55.5° C., 56° C., 56.5° C., 57° C., 57.5° C., 58° C., 58.5° C., 59° C., 59.5° C., 60° C., 60.5° C., 61° C., 61.5° C., 62° C., 62.5° C., 63° C., 63.5° C., 64° C., 64.5° C., 65° C., 65.5° C., 66° C., 66.5° C., 67° C., 67.5° C., 68° C., 68.5° C., 69° C., 69.5° C., or 70° C. Particularly, in any of the embodiments described in this paragraph, the polymerase can be Vent DNA polymerase, Vent (exo⁻) DNA polymerase, Deep Vent DNA polymerase, or Deep Vent (exo⁻) DNA polymerase.

In some embodiments, the polymerase is a Vent® DNA polymerase or a truncated or mutated version thereof, and the first temperature is selected from the range of about 70-80° C., and the second temperature is selected from the range of about 55-70° C. Specifically, in particular embodiments where the polymerase is a Vent® DNA polymerase, the first temperature is about 70° C., and the second temperature is selected from the range of about 55-70° C. In particular embodiments where the polymerase is a Vent® DNA polymerase, the first temperature is about 70.5° C., and the second temperature is selected from the range of about 55-70° C. In particular embodiments where the polymerase is a Vent® DNA polymerase, the first temperature is about 71° C., and the second temperature is selected from the range of about 55-70° C. In particular embodiments where the polymerase is a Vent® DNA polymerase, the first temperature is about 71.5° C., and the second temperature is selected from the range of about 55-70° C. In particular embodiments where the polymerase is a Vent® DNA polymerase, the first temperature is about 72° C., and the second temperature is selected from the range of about 55-70° C. In particular embodiments where the polymerase is a Vent® DNA polymerase, the first temperature is about 72.5° C., and the second temperature is selected from the range of about 55-70° C. In particular embodiments where the polymerase is a Vent® DNA polymerase, the first temperature is about 73° C., and the second temperature is selected from the range of about 55-70° C. In particular embodiments where the polymerase is a Vent® DNA polymerase, the first temperature is about 73.5° C., and the second temperature is selected from the range of about 55-70° C. In particular embodiments where the polymerase is a Vent® DNA polymerase, the first temperature is about 74° C., and the second temperature is selected from the range of about 55-70° C. In particular embodiments where the polymerase is a Vent® DNA polymerase, the first temperature is about 74.5° C., and the second temperature is selected from the range of about 55-70° C. In particular embodiments where the polymerase is a Vent® DNA polymerase, the first temperature is about 75° C., and the second temperature is selected from the range of about 55-70° C. In particular embodiments where the polymerase is a Vent® DNA polymerase, the first temperature is about 75.5° C., and the second temperature is selected from the range of about 55-70° C. In particular embodiments where the polymerase is a Vent® DNA polymerase, the first temperature is about 76° C., and the second temperature is selected from the range of about 55-70° C. In particular embodiments where the polymerase is a Vent® DNA polymerase, the first temperature is about 76.5° C., and the second temperature is selected from the range of about 55-70° C. In particular embodiments where the polymerase is a Vent® DNA polymerase, the first temperature is about 77° C., and the second temperature is selected from the range of about 55-70° C. In particular embodiments where the polymerase is a Vent® DNA polymerase, the first temperature is about 77.5° C., and the second temperature is selected from the range of about 55-70° C. In particular embodiments where the polymerase is a Vent® DNA polymerase, the first temperature is about 78° C., and the second temperature is selected from the range of about 55-70° C. In particular embodiments where the polymerase is a Vent® DNA polymerase, the first temperature is about 78.5° C., and the second temperature is selected from the range of about 55-70° C. In particular embodiments where the polymerase is a Vent® DNA polymerase, the first temperature is about 79° C., and the second temperature is selected from the range of about 55-70° C. In particular embodiments where the polymerase is a Vent® DNA polymerase, the first temperature is about 79.5° C., and the second temperature is selected from the range of about 55-70° C. In particular embodiments where the polymerase is a Vent® DNA polymerase, the first temperature is about 80° C., and the second temperature is selected from the range of about 55-80° C. Particularly, in any of the embodiments described in this paragraph, the first temperature selected from the range of about 70-80° C. can be about 70° C., 70.5° C., 71° C., 71.5° C., 72° C., 72.5° C., 73° C., 73.5° C., 74° C., 74.5° C., 75° C., 75.5° C., 76° C., 76.5° C., 77° C., 77.5° C., 78° C., 78.5° C., 79° C., 79.5° C., or 80° C. Particularly, in any of the embodiments described in this paragraph, the polymerase can be Vent DNA polymerase, Vent (exo⁻) DNA polymerase, Deep Vent DNA polymerase, or Deep Vent (exo⁻) DNA polymerase.

In some embodiments, the polymerase is a phi29 DNA Polymerase, and the first temperature is selected from the range of about 40-55° C., and the second temperature is selected from the range of about 20-37° C. Specifically, in particular embodiments where the polymerase is a phi29 DNA polymerase, the first temperature is about 40° C., and the second temperature is selected from the range of about 20-37° C. In particular embodiments where the polymerase is a phi29 DNA polymerase, the first temperature is about 40.5° C., and the second temperature is selected from the range of about 20-37° C. In particular embodiments where the polymerase is a phi29 DNA polymerase, the first temperature is about 41° C., and the second temperature is selected from the range of about 20-37° C. In particular embodiments where the polymerase is a phi29 DNA polymerase, the first temperature is about 41.5° C., and the second temperature is selected from the range of about 20-37° C. In particular embodiments where the polymerase is a phi29 DNA polymerase, the first temperature is about 42° C., and the second temperature is selected from the range of about 20-37° C. In particular embodiments where the polymerase is a phi29 DNA polymerase, the first temperature is about 42.5° C., and the second temperature is selected from the range of about 20-37° C. In particular embodiments where the polymerase is a phi29 DNA polymerase, the first temperature is about 43° C., and the second temperature is selected from the range of about 20-37° C. In particular embodiments where the polymerase is a phi29 DNA polymerase, the first temperature is about 43.5° C., and the second temperature is selected from the range of about 20-37° C. In particular embodiments where the polymerase is a phi29 DNA polymerase, the first temperature is about 44° C., and the second temperature is selected from the range of about 20-37° C. In particular embodiments where the polymerase is a phi29 DNA polymerase, the first temperature is about 44.5° C., and the second temperature is selected from the range of about 20-37° C. In particular embodiments where the polymerase is a phi29 DNA polymerase, the first temperature is about 45° C., and the second temperature is selected from the range of about 20-37° C. In particular embodiments where the polymerase is a phi29 DNA polymerase, the first temperature is about 45.5° C., and the second temperature is selected from the range of about 20-37° C. In particular embodiments where the polymerase is a phi29 DNA polymerase, the first temperature is about 46° C., and the second temperature is selected from the range of about 20-37° C. In particular embodiments where the polymerase is a phi29 DNA polymerase, the first temperature is about 46.5° C., and the second temperature is selected from the range of about 20-37° C. In particular embodiments where the polymerase is a phi29 DNA polymerase, the first temperature is about 47° C., and the second temperature is selected from the range of about 20-37° C. In particular embodiments where the polymerase is a phi29 DNA polymerase, the first temperature is about 47.5° C., and the second temperature is selected from the range of about 20-37° C. In particular embodiments where the polymerase is a phi29 DNA polymerase, the first temperature is about 48° C., and the second temperature is selected from the range of about 20-37° C. In particular embodiments where the polymerase is a phi29 DNA polymerase, the first temperature is about 48.5° C., and the second temperature is selected from the range of about 20-37° C. In particular embodiments where the polymerase is a phi29 DNA polymerase, the first temperature is about 49° C., and the second temperature is selected from the range of about 20-37° C. In particular embodiments where the polymerase is a phi29 DNA polymerase, the first temperature is about 49.5° C., and the second temperature is selected from the range of about 20-37° C. In particular embodiments where the polymerase is a phi29 DNA polymerase, the first temperature is about 50° C., and the second temperature is selected from the range of about 20-37° C. In particular embodiments where the polymerase is a phi29 DNA polymerase, the first temperature is about 50.5° C., and the second temperature is selected from the range of about 20-37° C. In particular embodiments where the polymerase is a phi29 DNA polymerase, the first temperature is about 51° C., and the second temperature is selected from the range of about 20-37° C. In particular embodiments where the polymerase is a phi29 DNA polymerase, the first temperature is about 51.5° C., and the second temperature is selected from the range of about 20-37° C. In particular embodiments where the polymerase is a phi29 DNA polymerase, the first temperature is about 52° C., and the second temperature is selected from the range of about 20-37° C. In particular embodiments where the polymerase is a phi29 DNA polymerase, the first temperature is about 52.5° C., and the second temperature is selected from the range of about 20-37° C. In particular embodiments where the polymerase is a phi29 DNA polymerase, the first temperature is about 53° C., and the second temperature is selected from the range of about 20-37° C. In particular embodiments where the polymerase is a phi29 DNA polymerase, the first temperature is about 53.5° C., and the second temperature is selected from the range of about 20-37° C. In particular embodiments where the polymerase is a phi29 DNA polymerase, the first temperature is about 54° C., and the second temperature is selected from the range of about 20-37° C. In particular embodiments where the polymerase is a phi29 DNA polymerase, the first temperature is about 54.5° C., and the second temperature is selected from the range of about 20-37° C. In particular embodiments where the polymerase is a phi29 DNA polymerase, the first temperature is about 55° C., and the second temperature is selected from the range of about 20-37° C. Particularly, in any of the embodiments described in this paragraph, the second temperature selected from the range of about 20-37° C. can be about 20° C., 20.5° C., 21° C., 21.5° C., 22° C., 22.5° C., 23° C., 23.5° C., 24° C., 24.5° C., 25° C., 25.5° C., 26° C., 26.5° C., 27° C., 27.5° C., 28° C., 28.5° C., 29° C., 29.5° C., 30° C., 30.5° C., 31° C., 31.5° C., 32° C., 32.5° C., 33° C., 33.5° C., 34° C., 34.5° C., 35° C., 35.5° C., 36° C., 36.5° C., or 37° C.

In some embodiments, the polymerase is a phi29 DNA polymerase, and the first temperature is selected from the range of about 40-55° C., and the second temperature is selected from the range of about 20-37° C. Specifically, in particular embodiments where the polymerase is a phi29 DNA polymerase, the first temperature is selected from the range of about 40-55° C., and the second temperature is about 20° C. In particular embodiments where the polymerase is a phi29 DNA polymerase, the first temperature is selected from the range of about 40-55° C., and the second temperature is about 20.5° C. In particular embodiments where the polymerase is a phi29 DNA polymerase, the first temperature is selected from the range of about 40-55° C., and the second temperature is about 21° C. In particular embodiments where the polymerase is a phi29 DNA polymerase, the first temperature is selected from the range of about 40-55° C., and the second temperature is about 21.5° C. In particular embodiments where the polymerase is a phi29 DNA polymerase, the first temperature is selected from the range of about 40-55° C., and the second temperature is about 22° C. In particular embodiments where the polymerase is a phi29 DNA polymerase, the first temperature is selected from the range of about 40-55° C., and the second temperature is about 22.5° C. In particular embodiments where the polymerase is a phi29 DNA polymerase, the first temperature is selected from the range of about 40-55° C., and the second temperature is about 23° C. In particular embodiments where the polymerase is a phi29 DNA polymerase, the first temperature is selected from the range of about 40-55° C., and the second temperature is about 23.5° C. In particular embodiments where the polymerase is a phi29 DNA polymerase, the first temperature is selected from the range of about 40-55° C., and the second temperature is about 24° C. In particular embodiments where the polymerase is a phi29 DNA polymerase, the first temperature is selected from the range of about 40-55° C., and the second temperature is about 24.5° C. In particular embodiments where the polymerase is a phi29 DNA polymerase, the first temperature is selected from the range of about 40-55° C., and the second temperature is about 25° C. In particular embodiments where the polymerase is a phi29 DNA polymerase, the first temperature is selected from the range of about 40-55° C., and the second temperature is about 25.5° C. In particular embodiments where the polymerase is a phi29 DNA polymerase, the first temperature is selected from the range of about 40-55° C., and the second temperature is about 26° C. In particular embodiments where the polymerase is a phi29 DNA polymerase, the first temperature is selected from the range of about 40-55° C., and the second temperature is about 26.5° C. In particular embodiments where the polymerase is a phi29 DNA polymerase, the first temperature is selected from the range of about 40-55° C., and the second temperature is about 27° C. In particular embodiments where the polymerase is a phi29 DNA polymerase, the first temperature is selected from the range of about 40-55° C., and the second temperature is about 27.5° C. In particular embodiments where the polymerase is a phi29 DNA polymerase, the first temperature is selected from the range of about 40-55° C., and the second temperature is about 28° C. In particular embodiments where the polymerase is a phi29 DNA polymerase, the first temperature is selected from the range of about 40-55° C., and the second temperature is about 28.5° C. In particular embodiments where the polymerase is a phi29 DNA polymerase, the first temperature is selected from the range of about 40-55° C., and the second temperature is about 29° C. In particular embodiments where the polymerase is a phi29 DNA polymerase, the first temperature is selected from the range of about 40-55° C., and the second temperature is about 29.5° C. In particular embodiments where the polymerase is a phi29 DNA polymerase, the first temperature is selected from the range of about 40-55° C., and the second temperature is about 30° C. In particular embodiments where the polymerase is a phi29 DNA polymerase, the first temperature is selected from the range of about 40-55° C., and the second temperature is about 30.5° C. In particular embodiments where the polymerase is a phi29 DNA polymerase, the first temperature is selected from the range of about 40-55° C., and the second temperature is about 31° C. In particular embodiments where the polymerase is a phi29 DNA polymerase, the first temperature is selected from the range of about 40-55° C., and the second temperature is about 31.5° C. In particular embodiments where the polymerase is a phi29 DNA polymerase, the first temperature is selected from the range of about 40-55° C., and the second temperature is about 32° C. In particular embodiments where the polymerase is a phi29 DNA polymerase, the first temperature is selected from the range of about 40-55° C., and the second temperature is about 32.5° C. In particular embodiments where the polymerase is a phi29 DNA polymerase, the first temperature is selected from the range of about 40-55° C., and the second temperature is about 33° C. In particular embodiments where the polymerase is a phi29 DNA polymerase, the first temperature is selected from the range of about 40-55° C., and the second temperature is about 33.5° C. In particular embodiments where the polymerase is a phi29 DNA polymerase, the first temperature is selected from the range of about 40-55° C., and the second temperature is about 34° C. In particular embodiments where the polymerase is a phi29 DNA polymerase, the first temperature is selected from the range of about 40-55° C., and the second temperature is about 34.5° C. In particular embodiments where the polymerase is a phi29 DNA polymerase, the first temperature is selected from the range of about 40-55° C., and the second temperature is about 35° C. In particular embodiments where the polymerase is a phi29 DNA polymerase, the first temperature is selected from the range of about 40-55° C., and the second temperature is about 35.5° C. In particular embodiments where the polymerase is a phi29 DNA polymerase, the first temperature is selected from the range of about 40-55° C., and the second temperature is about 36° C. In particular embodiments where the polymerase is a phi29 DNA polymerase, the first temperature is selected from the range of about 40-55° C., and the second temperature is about 36.5° C. In particular embodiments where the polymerase is a phi29 DNA polymerase, the first temperature is selected from the range of about 40-55° C., and the second temperature is about 37° C. Particularly, in any of the embodiments described in this paragraph, the first temperature selected from the range of about 40-55° C. can be about 40° C., 40.5° C., 41° C., 41.5° C., 42° C., 42.5° C., 43° C., 43.5° C., 44° C., 44.5° C., 45° C., 45.5° C., 46° C., 46.5° C., 47° C., 47.5° C., 48° C., 48.5° C., 49° C., 49.5° C., 50° C., 50.5° C., 51° C., 51.5° C., 52° C., 52.5° C., 53° C., 53.5° C., 54° C., 54.5° C., or 55° C.

In some embodiments, the pair of primers are configured to amplify a region in a target nucleic acid molecule that is less than 100 bp long. In some embodiments, the amplicon produced by the present method is less than 90 bp long. In some embodiments, the amplicon produced by the present method is less than 80 bp long. In some embodiments, the amplicon produced by the present method is less than 70 bp long. In some embodiments, the amplicon produced by the present method is less than 60 bp long. In some embodiments, the amplicon produced by the present method is less than 50 bp long. In some embodiments, the amplicon produced by the present method is about 20-50 bp long. In some embodiments, the amplicon produced by the present method is about 30-50 bp long. In some embodiments, the amplicon produced by the present method is about 35-50 bp long.

In some embodiments, to reduce time needed for primer extension, the pair of primers are configured to produce a short amplicon of about 20 base pair (bp) to about 50 bp in length. The amplicon comprises at least a central portion that corresponds to a unique sequence in the target nucleic acid molecule, which central portion may be flanked by primer sequences that are either the same as or different from sequences in the target molecule. For example, in specific embodiments, the amplicon is about 20 bp in length. In specific embodiments, the amplicon is about 21 bp in length. In specific embodiments, the amplicon is about 22 bp in length. In specific embodiments, the amplicon is about 23 bp in length. In specific embodiments, the amplicon is about 24 bp in length. In specific embodiments, the amplicon is about 25 bp in length. In specific embodiments, the amplicon is about 26 bp in length. In specific embodiments, the amplicon is about 27 bp in length. In specific embodiments, the amplicon is about 28 bp in length. In specific embodiments, the amplicon is about 29 bp in length. In specific embodiments, the amplicon is about 30 bp in length. In specific embodiments, the amplicon is about 31 bp in length. In specific embodiments, the amplicon is about 32 bp in length. In specific embodiments, the amplicon is about 33 bp in length. In specific embodiments, the amplicon is about 34 bp in length. In specific embodiments, the amplicon is about 35 bp in length. In specific embodiments, the amplicon is about 36 bp in length. In specific embodiments, the amplicon is about 37 bp in length. In specific embodiments, the amplicon is about 38 bp in length. In specific embodiments, the amplicon is about 39 bp in length. In specific embodiments, the amplicon is about 40 bp in length. In specific embodiments, the amplicon is about 41 bp in length. In specific embodiments, the amplicon is about 42 bp in length. In specific embodiments, the amplicon is about 43 bp in length. In specific embodiments, the amplicon is about 44 bp in length. In specific embodiments, the amplicon is about 45 bp in length. In specific embodiments, the amplicon is about 46 bp in length. In specific embodiments, the amplicon is about 47 bp in length. In specific embodiments, the amplicon is about 48 bp in length. In specific embodiments, the amplicon is about 49 bp in length. In specific embodiments, the amplicon is about 50 bp in length.

In some embodiments, the amplicon has a melting temperature (Tₘ or Tₘ value) that is equal to or lower than about 90° C. In some embodiments, the amplicon has a Tₘ value that is equal to or lower than about 89° C. In some embodiments, the amplicon has a Tₘ value that is equal to or lower than about 88° C. In some embodiments, the amplicon has a Tₘ value that is equal to or lower than about 87° C. In some embodiments, the amplicon has a Tₘ value that is equal to or lower than about 86° C. In some embodiments, the amplicon has a Tₘ value that is equal to or lower than about 85° C. In some embodiments, the Tₘ value of the amplicon is determined using a computer algorithm based on the sequence of the amplicon. In some embodiments, the Tₘ value of the amplicon is determined using a computer algorithm based on the sequence of the amplicon and one or more other conditions of the amplification mixture, such as but are not limited the concentration of Na⁺, the concentration of Mg²⁺, or the concentration of nucleic acid molecules in the amplification mixture. In some embodiments, the computer algorithm is selected from NUPACK web tool (www.nupack.org), DNAMelt Web (http://unafold.ma.albany.edu/?q=DINAMelt), NOVOPRO www.novopro.cn/tools/rev_comp.html), the BLAST algorithm at the NCBI website (www.ncbi.nlm.nih.gov/tools/primer-blast), Primer Premier (Premier Biosoft Inc., Canada), AlignMiner (http://www.scbi.uma.es/alignminer/), Oligo (DBA Oligo, Inc., CO, US), PerlPrimer (http://perlprimer.sourceforge.net/), Primer3Web (http://bioinfo.ut.ee/primer3/) and DNAstar (DNASTAR Inc., WI, US).

In some embodiments, the relatively short amplicon size makes it possible to conduct the amplification reaction by swiftly changing the reaction temperature between the first and second temperature, producing detectable amount of amplicon in less than 15 minutes.

Particularly, in some embodiments, the present method comprises subjecting an amplification mixture to swift thermal cycles between the first and the second temperatures, where each thermal cycle is less than about 20 s, more particularly less than about 15 s, more particularly less than about 10 s, more particularly less than about 8 s, more particularly less than about 6 s, more particularly less than about 5 s, more particularly less than about 4 s, more particularly less than about 3 s, more particularly less than about 2 s, more particularly less than about 1 s, more particularly less than about 0.5 s, or more particularly less than about 0.1 s.

In some embodiments, during each thermal cycle, the amplification mixture is incubated at the first temperature for no more than 5 s, and then incubated at the second temperature for no more than 5 s. In some embodiments, during each thermal cycle, the amplification mixture is incubated at the first temperature for no more than 2 s, and then incubated at the second temperature for no more than 2 s. In some embodiments, during each thermal cycle, the amplification mixture is incubated at the first temperature for less than about 1 s, and then incubated at the second temperature for less than about 1 second. In some embodiments, during each thermal cycle, the amplification mixture is incubated at the first temperature for about 0.5 second, and then incubated at the second temperature for about 0.5 second. In some embodiments, during each thermal cycle, the amplification mixture is incubated at the first temperature for about 0.1 second, and then incubated at the second temperature for about 0.1 second.

In some embodiments, the time to complete each thermal cycle is longer than the sum of the time for incubating at the first temperature and the time for incubating at the second temperature, as time is needed to ramp the reaction temperatures between the two temperatures, and such time gap is referred herein as the “ramp time.” According to the present disclosure, the total ramp time in a thermal cycle includes the time taken to decrease the reaction temperature from the first temperature to the second temperature, as well as the time taken to increase the reaction temperature from the second temperature to the first temperature. In some embodiments, the total ramp time in a thermal cycle is less than about 10 s. In some embodiments, the total ramp time in a thermal cycle is less than about 5 s. In some embodiments, the total ramp time in a thermal cycle is less than about 2 s. In some embodiments, the total ramp time in a thermal cycle is less than about 1 s. In some embodiments, the total ramp time in a thermal cycle is less than about 0.5 s. In an exemplary embodiment, as demonstrated in Example 9, the present method, performed using a microfluidic platform having a ramp speed of 8° C./s, produced detectable specific amplification in less than 8 seconds (40 thermal cycles). Additional exemplary methods and instruments that can be used in connection with the present methods and systems are provided below.

Early work in the early 1990s established the feasibility of rapid cycling using capillary tubes and hot air for temperature control. Over the past 20 years, abundant efforts have been deployed in the field to improve PCR instruments, particularly the ability of thermal cyclers in rapidly and precisely controlling and monitoring reaction temperatures, according to the paradigm of PCR protocols. Various methods and technologies have been used to avoid or reduce delays due to thermal transfer through the walls of conical tubes, low surface area-to-volume ratios, or heating of large volume samples. Improved thermal conductive materials and designs of reaction chamber as well as heating elements have been used to further reduce the ramping time.

In some embodiments, the number of thermal cycles performed by the present method is about 20 to 50 cycles. In some embodiments, the number of thermal cycles performed by the present method is at least 20 cycles. In some embodiments, the number of thermal cycles performed by the present method is at least 25 cycles. In some embodiments, the number of thermal cycles performed by the present method is at least 30 cycles. In some embodiments, the number of thermal cycles performed by the present method is at least 35 cycles. In some embodiments, the number of thermal cycles performed by the present method is at least 40 cycles. In some embodiments, the number of thermal cycles performed by the present method is at least 45 cycles. In some embodiments, the number of thermal cycles performed by the present method is at least 50 cycles.

In some embodiments, the total reaction time of the present method is about 2-20 minutes. In some embodiments, the total reaction time of the present method is less than about 20 minutes. In some embodiments, the total reaction time of the present method is less than about 15 minutes. In some embodiments, the total reaction time of the present method is less than about 10 minutes. In some embodiments, the total reaction time of the present method is less than about 7 minutes. In some embodiments, the total reaction time of the present method is less than about 5 minutes. In some embodiments, the total reaction time of the present method is less than about 2 minutes.

In some embodiments, the volume of the amplification mixture is selected from the range of about 1 to 30 μL. In some embodiments, the amplification mixture is 1 μL. In some embodiments, the amplification mixture is 2 μL. In some embodiments, the amplification mixture is 3 μL. In some embodiments, the amplification mixture is 4 μL. In some embodiments, the amplification mixture is 5 μL. In some embodiments, the amplification mixture is 6 μL. In some embodiments, the amplification mixture is 7 μL. In some embodiments, the amplification mixture is 8 μL. In some embodiments, the amplification mixture is 9 μL. In some embodiments, the amplification mixture is 10 μL. In some embodiments, the amplification mixture is 15 μL. In some embodiments, the amplification mixture is 20 μL. In some embodiments, the amplification mixture is 25 μL. In some embodiments, the amplification mixture is 30 μL. In some embodiments, the present method is performed using a microfluidic device. In some embodiments, the present method is performed in droplets of amplification mixtures.

Various types of samples can be used in connection with the present disclosure, including but not limited to biological samples separated from a subject (such as a blood sample, a saliva sample, oral or nasal swab), samples containing nucleic acid molecules isolated or extracted from a biological sample, or samples containing synthetic nucleic acid molecules. In some embodiments, the target nucleic acid is DNA. In some embodiments, the target nucleic acid is RNA. The present disclosure contemplates and demonstrates that the present methods and kits can be used to amplify and detect trace amount of target nucleic acid molecules present in a sample. In specific embodiments, the amplification mixture contains less than 1.0×10⁻¹² M target nucleic acid. In specific embodiments, the amplification mixture contains less than 1.0×10⁻¹³ M target nucleic acid. In specific embodiments, the amplification mixture contains less than 1.0×10⁻¹⁴ M target nucleic acid. In specific embodiments, the amplification mixture contains no more than 1.0×10⁻¹⁵ M target nucleic acid. In specific embodiments, the amplification mixture contains no more than 1.0×10⁻¹⁶ M target nucleic acid. In specific embodiments, the amplification mixture contains no more than 1.0×10⁻¹⁷ M target nucleic acid. In specific embodiments, the amplification mixture contains no more than 1.0×10⁻¹⁸ M target nucleic acid.

In specific embodiments, the amplification mixture contains less than 1.0×10⁷ copies of the target nucleic acid molecule. In specific embodiments, the amplification mixture contains less than 1.0×10⁶ copies of the target nucleic acid molecule. In specific embodiments, the amplification mixture contains less than 1.0×10⁵ copies of the target nucleic acid molecule. In specific embodiments, the amplification mixture contains less than 1.0×10⁴ copies of the target nucleic acid molecule. In specific embodiments, the amplification mixture contains less than 1.0×10³ copies of the target nucleic acid molecule. In specific embodiments, the amplification mixture contains less than 100 copies of the target nucleic acid molecule. In specific embodiments, the amplification mixture contains less than 10 copies of the target nucleic acid molecule.

As would be appreciated by those of ordinary skill in the art, the present methods and systems can detect a trace amount of target nucleic acid present in a sample. To prevent possible contamination of the reaction by nucleic acid molecules floating in the ambient air, in some embodiments, the present method further comprises steps that prevents or reduces the effect of possible contamination.

Uracil-DNA Glycosylase (UDG) is an uracil-DNA glycosylase that catalyzes the hydrolysis of the N-glycosylic bond between uracil and sugar, releasing free uracil, and leaving an apyrimidinic site in uracil-containing single or double-stranded DNA, which is easily broken by hydrolysis. UDG is active on single- and double-stranded uracil (dU)-containing DNA, while dUTPs is not a substrate for UDG. UDG can be used to specifically degrade nucleic acids that are produced by prior amplification reactions, a common source of carry-over contamination. In some embodiments, UDG allows previous amplification products, or mis-primed, nonspecific products to degrade, leaving native nucleic acid templates intended for amplification intact. Accordingly, in some embodiments, the amplification mixture comprises dUTPs for performing the amplification reaction. In specific embodiments, the amplification mixture comprises Uracil-DNA Glycosylase (UDG) in the amplification mixture for performing the reaction. In specific embodiments, the amplification mixture comprises both dUTPs and UDG in the amplification mixture for performing the reaction. In some embodiments where dUTPs is used for amplification, the amplification mixture does not also include dTTPs.

Primers described herein, such as those described in Section 4.3 and those designed according to the exemplary procedures described in Example 7, can be used in connection with the present method. Particularly, in some embodiments, the amplification reaction contains a pair of primer configured to define an amplification region of about 20-50 bp in the target nucleic acid molecule. Particularly, in some embodiments, at least one primer presents in the amplification mixture at the concentration of no less than 1.0×10⁻⁶M. In some embodiments, at least one primer presents in the amplification mixture at the concentration of no less than 1.5×10⁻⁶M. In some embodiments, at least one primer presents in the amplification mixture at the concentration of no less than 2.0×10⁻⁶M. In some embodiments, at least one primer presents in the amplification mixture at the concentration of no less than 2.5×10⁻⁶M. In some embodiments, at least one primer presents in the amplification mixture at the concentration of no less than 3.0×10⁻⁶M. In some embodiments, both primers present in the amplification mixture at the concentration of no less than 1.0×10⁻⁶M. In some embodiments, both primers present in the amplification mixture at the concentration of no less than 1.5×10⁻⁶M. In some embodiments, both primers present in the amplification mixture at the concentration of no less than 2.0×10⁻⁶M. In some embodiments, both primers present in the amplification mixture at the concentration of no less than 2.5×10⁻⁶M. In some embodiments, both primers present in the amplification mixture at the concentration of no less than 3×10⁻⁶M.

Particularly, in some embodiments, the melting temperature (Tₘ or Tₘ value) of a primer is within ±6° C. of the second temperature employed in the method. In some embodiments, the Tₘ value of a primer is within ±5° C. of the second temperature employed in the method. In some embodiments, the Tₘ value of a primer is within ±4° C. of the second temperature employed in the method. In some embodiments, the Tₘ value of a primer is within ±3° C. of the second temperature employed in the method. In some embodiments, the Tₘ value of a primer is within ±2° C. of the second temperature employed in the method. In some embodiments, the Tₘ value of a primer is within ±1° C. of the second temperature employed in the method. In some embodiments, the Tₘ value of a primer is within ±0.5° C. of the second temperature employed in the method. In some embodiments, Tₘ values of both primers are within ±6° C. of the second temperature employed in the method. In some embodiments, Tₘ values of both primers are within ±5° C. of the second temperature employed in the method. In some embodiments, Tₘ values of both primers are within ±4° C. of the second temperature employed in the method. In some embodiments, Tₘ values of both primers are within ±3° C. of the second temperature employed in the method. In some embodiments, Tₘ values of both primers are within ±2° C. of the second temperature employed in the method. In some embodiments, Tₘ values of both primers are within ±1° C. of the second temperature employed in the method. In some embodiments, Tₘ values of both primers are within ±0.5° C. of the second temperature employed in the method.

In some embodiments, the first temperature employed in the method is within ±6° C. of the Tₘ value of the amplicon. In some embodiments, the first temperature employed in the method is within ±5° C. of the Tₘ value of the amplicon. In some embodiments, the first temperature employed in the method is within ±4° C. of the Tₘ value of the amplicon. In some embodiments, the first temperature employed in the method is within ±3° C. of the Tₘ value of the amplicon. In some embodiments, the first temperature employed in the method is within ±2° C. of the Tₘ value of the amplicon. In some embodiments, the first temperature employed in the method is within ±1° C. of the Tₘ value of the amplicon. In some embodiments, the first temperature employed in the method is within ±0.5° C. of the Tₘ value of the amplicon. In some embodiments, the first temperature employed in the method is about the same as the Tₘ value of the amplicon. In particular embodiments described in this paragraph, the second temperature employed in the method is within ±6° C. of the Tₘ value of at least one primer. In particular embodiments described in this paragraph, the second temperature employed in the method is within ±6° C., within ±5° C., within ±4° C., within ±3° C., within ±2° C., within ±1° C., or within ±0.5° C. of the Tₘ value of at least one primer used in the method. In particular embodiments described in this paragraph, the second temperature employed in the method is about the same as the Tm value of at least one primer used in the method. In particular embodiments described in this paragraph, the Tₘ values of a pair of primers are about the same. In particular embodiments described in this paragraph, the Tₘ values of a pair of primers differ from each other by less than about 3° C., about 2° C., about 1° C. or about 0.5° C.

In some embodiments, the pair of primers employed in the method each has a melting temperature. The average of the two Tₘ values of the pair of primers is referred to as the average melting temperature of the pair of primers. Particularly, in some embodiments, the second temperature employed in the method is within ±6° C. of the average melting temperature of the pair of primers used in the method. In some embodiments, the second temperature employed in the method is within ±5° C. of the average melting temperature of the pair of primers used in the method. In some embodiments, the second temperature employed in the method is within ±4° C. of the average melting temperature of the pair of primers used in the method. In some embodiments, the second temperature employed in the method is within ±3° C. of the average melting temperature of the pair of primers used in the method. In some embodiments, the second temperature employed in the method is within ±2° C. of the average melting temperature of the pair of primers used in the method. In some embodiments, the second temperature employed in the method is within ±1° C. of the average melting temperature of the pair of primers used in the method. In some embodiments, the second temperature employed in the method is within ±0.5° C. of the average melting temperature of the pair of primers used in the method. In some embodiments, the second temperature employed in the method is about the same as the average melting temperature of the pair of primers used in the method. In particular embodiments described in this paragraph, the Tₘ values of a pair of primers are about the same. In particular embodiments described in this paragraph, the Tₘ values of a pair of primers differ from each other by less than about 3° C., about 2° C., about 1° C. or about 0.5° C. In particular embodiments described in this paragraph, the first temperature employed in the method is within ±6° C., within ±5° C., within ±4° C., within ±3° C., within ±2° C., within ±1° C., or within ±0.5° C. of the Tₘ value of the amplicon. In particular embodiments described in this paragraph, the first temperature employed in the method is about the same as the Tₘ value of the amplicon.

Methods for determining Tₘ values of nucleic acids (e.g., an oligonucleotide primer, or an amplicon) are known in the art. For example, various computer algorithms capable of determining Tₘ values based on the nucleic acid sequence and/or environment condition (e.g., salt concentration) are known in the art. Exemplary computer algorithms or software that can be used to determining Tₘ values for nucleic acids include but are not limited to NUPACK web tool (www.nupack.org), DNAMelt Web (http://unafold.ma.albany.edu/?q=DINAMelt), NOVOPRO www.novopro.cn/tools/rev_comp.html), the BLAST algorithm at the NCBI website (www.ncbi.nlm.nih.gov/tools/primer-blast), Primer Premier (Premier Biosoft Inc., Canada), AlignMiner (http://www.scbi.uma.es/alignminer/), Oligo (DBA Oligo, Inc., CO, US), PerlPrimer (http://perlprimer.sourceforge.net/), Primer3Web (http://bioinfo.ut.ee/primer3/) and DNAstar (DNASTAR Inc., WI, US). In some embodiments, computer algorithms or software that can be used to determining Tₘ values for nucleic acids is selected from NUPACK web tool (www.nupack.org), DNAMelt Web (http://unafold.ma.albany.edu/?q=DINAMelt), NOVOPRO www.novopro.cn/tools/rev_comp.html) and the BLAST algorithm at the NCBI website (www.ncbi.nlm.nih.gov/tools/primer-blast).

In some embodiments, the present method further comprises determining the Tₘ value of the amplicon to be produced by the method. In some embodiments, the present method further comprises determining the Tₘ value of at least one primer to be used in the method. In some embodiments, the present method further comprises determining the Tₘ values for both primers to be used in the method. In some embodiments, the present method further comprises determining the Tₘ value of both primers to be used in the method and further comprises determining the average melting temperature of the pair of primers to be used in the method. In some embodiments, the Tₘ value of a primer or amplicon is determined using a computer algorithm based on the sequence of the primer or the amplicon. In some embodiments, the Tₘ value of a primer or amplicon is determined using a computer algorithm based on the sequence of the primer or the amplicon and one or more other conditions of the amplification mixture, such as but are not limited the concentration of Na⁺, the concentration of Mg²⁺, or the concentration of nucleic acid molecules in the amplification mixture.

Polymerases as described here, such as those described in Section 4.4, can be used in connection with the present method. Particularly, in some embodiments, the polymerase is a thermostable polymerase as described herein. In some embodiments, the amplification mixture contains the polymerase at the concentration of no less than 0.1 U/μL. In some embodiments, the amplification mixture contains the polymerase at the concentration of no less than 0.2 U/μL. In some embodiments, the amplification mixture contains the polymerase at the concentration of no less than 0.3 U/μL. In some embodiments, the amplification mixture contains the polymerase at the concentration of no less than 0.4 U/μL. In some embodiments, the amplification mixture contains the polymerase at the concentration of no less than 0.5 U/μL. In some embodiments, the amplification mixture contains the polymerase at the concentration of no less than 1 U/μL.

In some embodiments, the polymerase has an optimal temperature between the first and second temperature employed in the present method. In some embodiments, the polymerase has an optimal temperature within ±6° C. of the second temperature employed in the method. In some embodiments, the polymerase has an optimal temperature within ±5° C. of the second temperature employed in the method. In some embodiments, the polymerase has an optimal temperature within ±4° C. of the second temperature employed in the method. In some embodiments, the polymerase has an optimal temperature within ±3° C. of the second temperature employed in the method. In some embodiments, the polymerase has an optimal temperature within ±2° C. of the second temperature employed in the method. In some embodiments, the polymerase has an optimal temperature within ±1° C. of the second temperature employed in the method. In some embodiments, the polymerase has an optimal temperature within ±0.5° C. of the second temperature employed in the method.

In some embodiments, the polymerase has an optimal temperature within ±5° C. of the first temperature employed in the method. In some embodiments, the polymerase has an optimal temperature within ±4° C. of the first temperature employed in the method. In some embodiments, the polymerase has an optimal temperature within ±3° C. of the first temperature employed in the method. In some embodiments, the polymerase has an optimal temperature within ±2° C. of the first temperature employed in the method. In some embodiments, the polymerase has an optimal temperature within ±1° C. of the first temperature employed in the method. In some embodiments, the polymerase has an optimal temperature within ±0.5° C. of the first temperature employed in the method.

In a specific embodiment, the present method for amplifying a target nucleic acid molecule in a sample comprises contacting a Bst DNA polymerase and a pair of oligonucleotide primers with the sample, thereby forming an amplification mixture; subjecting the amplification mixture through a number of thermal cycles between a first temperature selected from about 76° C., about 75° C., about 74° C., about 73° C., about 72° C., and a second temperature selected from about 61° C., about 62° C., about 63° C., about 64° C., and about 65° C., where each thermal cycle comprises incubating the amplification mixture at the first temperature for no more than 1 s and incubating the amplification at the second temperature for no more than 1 s, and a total ramp time of no more than 2 s, thereby producing an amplicon of about 20-50 base pair (bp) in length in less than 10 minutes. Particularly, in this embodiment, the target nucleic acid presents in the sample in a concentration of less than 1.0×10⁻¹⁴M. More specifically, in this embodiment, the target nucleic acid concentration in the sample is less than 1.0×10⁻¹⁵M, less than 1.0×10⁻¹⁶M, less than 1.0×10⁻¹⁷M or less than 1.0×10⁻¹⁸M. Particularly, in this embodiment, the target nucleic acid presents in the sample in a concentration of less than 1.0×10⁵ copies. More specifically, in this embodiment, the target nucleic acid concentration in the sample is less than 1.0×10⁴ copies, less than 1.0×10³ copies, less than 100 copies or less than 10 copies.

Amplicon produced by the present method can be detected using methods known in the art, such as fluorescent detection, colorimetric detection and electrophoresis detection. Conventional methods for real-time monitoring PCR amplification can be also used for real-time monitoring of amplification using the present methods. Particularly, in some embodiments, the amount of amplicon produced is measured during each thermal cycle. In other embodiments, the amount of amplicon produced is measured every 2, 5 or 10 thermal cycles. The amplicons can be purified from the amplification mixture and subjected to sequence analysis, such as next-generation sequencing, to identify the sequence, source and nature of the target nucleic acid molecule.

Such detection and analysis of the amplicon can be further used as the basis for various analysis and diagnosis relating to the target nucleic acid and the source thereof (such as a biological sample containing the target nucleic acid and a subject providing such biological sample). As a non-limiting example, methods and kits disclosed herein can be used for detecting presence of a pathogen in a biological sample. For example, the method and kit can employ primers configured to define an amplification region in the pathogen's genome having a unique sequence, and detect the presence of the unique sequence in the biological sample. Such methods can be applied to, for example, diagnosis of an infectious disease in a patient caused by the pathogen, detection of adulteration or contamination in a biological sample by the pathogen, quality control for food and beverage, etc. Another non-limiting example, methods and kits disclosed herein can be used for detecting a genetic alteration in a subject. Particularly useful scenarios include but are not limited detection or single nucleotide polymorphism in a subject and genetic diseases attributed to point mutations. For example, the methods and kit can employ primers configured to define an amplification region in the genomic sequence that is known or prone to have such a mutation, and detect the presence of the mutation by subjecting the amplicon to sequencing analysis. Many other possible application of the methods and kits disclosed herein will become immediately apparent to those of ordinary skill in the art upon reading the present disclosure, and such additional uses and applications are also contemplated by, and included in, the present disclosure.

Accordingly, in another aspect, provided herein is a method of detecting a target nucleic acid in a sample, comprising contacting a polymerase and a pair of oligonucleotide primers with the sample, thereby forming an amplification mixture; subjecting the amplification mixture to a number of thermal cycles between a first temperature and a second temperature, thereby amplifying at least a portion of the target nucleic acid through polymerase chain reaction; and detecting the presence or absence of an amplicon in the amplification mixture. In some embodiments, the first temperature is selected from the range of about 68° C. to 78° C. In some embodiments, the second temperature is selected from the range of about 55° C. to 69° C. In some embodiments, the pair of oligonucleotide primers are configured to produce an amplicon that is about 20-50 bp long. In some embodiments, the polymerase is selected from a Bst DNA polymerase, a DNA Polymerase I, Large (Klenow) Fragment, and Vent® DNA Polymerase, or a mutated or truncated form thereof. In some embodiments, the amplification mixture further contains dNTPs and polyethylene glycol. In some embodiments, the detecting of the amplicon is performed by fluorescent detection or colorimetric detection, or other methods known in the art. For example, in some embodiments, the present method further provides real-time monitoring of the amplification. Particularly, in some embodiments, the amount of amplicon produced is measured during each thermal cycle. In other embodiments, the amount of amplicon produced is measured every 2, 5 or 10 thermal cycles. Detection and measurement of the amount of amplicon produced can be achieved using conventional methods for real-time monitoring PCR amplification.

In another aspect, provided herein is a method for diagnosing an infection by a pathogen in a subject, comprising providing a nucleic acid containing sample collected from the subject; contacting a polymerase and a pair of oligonucleotide primers with the sample, thereby forming an amplification mixture, the pair of oligonucleotide primers configured to amplify an unique sequence in the genome of the pathogen; subjecting the amplification mixture to a number of thermal cycles between a first temperature and a second temperature, thereby producing an amplicon through polymerase chain reaction; and detecting the presence or absence of the amplicon in the amplification mixture. In some embodiments, the first temperature is selected from the range of about 68° C. to 78° C. In some embodiments, the second temperature is selected from the range of about 55° C. to 69° C. In some embodiments, the amplicon that is about 20-50 bp long. In some embodiments, the polymerase is selected from a Bst DNA polymerase, a DNA Polymerase I, Large (Klenow) Fragment, and Vent® DNA Polymerase, or a mutated or truncated form thereof. In some embodiments, the amplification mixture further contains dNTPs and polyethylene glycol. In some embodiments, the sample contains extracted genomic nucleic acid of the subject. In some embodiments, the sample contains cell-free nucleic acid molecules from the subject. In some embodiments, the sample is a bodily fluid sample. In some embodiments, the pathogen is a microbial organism, such as a virus, bacteria or fungi. In some embodiments, the pathogen is a parasite, such as a protozoa, helminths or ectoparasites.

In another aspect, provided herein is a method for detecting a genetic alteration in a subject comprising providing a sample from the subject; contacting a polymerase and a pair of oligonucleotide primers with the sample, thereby forming an amplification mixture, the pair of oligonucleotide primers configured to amplify a target sequence having or suspected of having the genetic alteration; subjecting the amplification mixture to a number of thermal cycles between a first temperature and a second temperature, thereby producing an amplicon through polymerase chain reaction; and sequencing the amplicon to determine the presence of absence of the genetic alteration. In some embodiments, the first temperature is selected from the range of about 68° C. to 78° C. In some embodiments, the second temperature is selected from the range of about 55° C. to 69° C. In some embodiments, the amplicon that is about 20-50 bp long. In some embodiments, the polymerase is selected from a Bst DNA polymerase, a DNA Polymerase I, Large (Klenow) Fragment, and Vent® DNA Polymerase, or a mutated or truncated form thereof. In some embodiments, the amplification mixture further contains dNTPs and polyethylene glycol. In some embodiments, the genetic alteration is a gene mutation, such as insertion, deletion, substitution, or copy number variation. In some embodiments, the genetic alteration is single nucleotide polymorphism. In some embodiments, the method further comprises diagnosis or prognosis of a genetic condition associated with the genetic alteration.

### 4.6 Kits

In another aspect of the present disclosure, provided herein is also a kit for performing the present methods. The kit comprises a plurality of components either mixed together in an amplification mixture or contained in at least two separate containers. In some embodiments, the kit comprises a polymerase and a pair of nucleotide primers. Primers provided herein, such as those described in Section 4.3 and those designed according to the exemplary procedures described in Example 7, and polymerases provided herein, such as those described in Section 4.4, can be used in connection with the present kit.

In some embodiments, the kit further comprises dNTPs and a buffer solution suitable for the polymerase. A buffer solution provides ion concentration, pH and/or coenzymes that facilitates the activity of the polymerase. Methods for selecting and making a buffer solution suitable for a particular polymerase are known in the art. For example, commercially available polymerases are typically sold with recommended recipe for a suitable buffer solution. In some embodiments, the kit further comprises polyethylene glycol (PEG). In some embodiments, the polyethylene glycol is PEG 200, PEG 400, PEG 2000 or PEG 4000. In some embodiments, the kit further comprises glycerol.

In some embodiments, the kit further comprises a reagent capable of facilitating the unwinding of double strands near the position where the primer anneals in the target nucleic acid. A particularly useful agent is a single strand binding protein (SSB). In some embodiments, the SSB is a stable and active in the temperature range where the present method is performed. In particular embodiments, the SSB is derived from a microbial organism, such as a bacteria or phage. In specific embodiments, the present kit comprises SSB selected from the T4 phage 32 SSB, T7 phage 2.5 SSB, phi phage 29 SSB, or E. coli SSB.

In some embodiments, the kit further comprises reagents for detecting and quantifying the amplicon produced, such as a fluorescent dye or a pH indicator. Suitable reagents for this purpose are known in the art. For example, certain fluorescent dye (e.g., Evagreen) emits a stronger fluorescent signal upon binding to double-stranded amplification product, and the measuring the strength of the fluorescent signal emitted from the amplification reaction is indicative of the amount of amplicon produced.

In some embodiments, the kit further comprises instructions for using the kit. For example, in some embodiments, various components of the kit are provided in the form of a mixture, and the kit comprises an instruction for adding a suitable amount of sample to form an amplification mixture. Alternatively, in some embodiments, various components of the kit are provided in at least two separate containers, and the kit comprises an instruction of mixing the components in the separate containers and a suitable amount of sample to form the amplification mixture.

In specific embodiments, the instruction specifies that the amplification mixture comprises the polymerase at a concentration of no less than 0.1 U/μL. In specific embodiments, the instruction specifies that the amplification mixture comprises the polymerase at a concentration of no less than 0.2 U/μL. In specific embodiments, the instruction specifies that the amplification mixture comprises the polymerase at a concentration of no less than 0.3 U/μL. In specific embodiments, the instruction specifies that the amplification mixture comprises the polymerase at a concentration of no less than 0.4 U/μL. In specific embodiments, the instruction specifies that the amplification mixture comprises the polymerase at a concentration of no less than 0.5 U/μL. In specific embodiments, the instruction specifies that the amplification mixture comprises the polymerase at a concentration of no less than 1 U/μL.

In specific embodiments, the instruction specifies that the amplification mixture comprises at least one of the primers at a concentration of no less than 1.0×10⁻⁶M. In specific embodiments, the instruction specifies that the amplification mixture comprises at least one of the primers at a concentration of no less than 1.5×10⁻⁶ M. In specific embodiments, the instruction specifies that the amplification mixture comprises at least one of the primers at a concentration of no less than 2.0×10⁻⁶ M. In specific embodiments, the instruction specifies that the amplification mixture comprises at least one of the primers at a concentration of no less than 2.5×10⁻⁶ M. In specific embodiments, the instruction specifies that the amplification mixture comprises at least one of the primers at a concentration of no less than 3.0×10⁻⁶ M.

In specific embodiments, the instruction specifies that the amplification mixture comprises both primers at the concentration of no less than 1.0×10⁻⁶M each. In specific embodiments, the instruction specifies that the amplification mixture comprises both primers at the concentration of no less than 1.5×10⁻⁶M each. In specific embodiments, the instruction specifies that the amplification mixture comprises both primers at the concentration of no less than 2.0×10⁻⁶ M each. In specific embodiments, the instruction specifies that the amplification mixture comprises both primers at the concentration of no less than 2.5×10⁻⁶M each. In specific embodiments, the instruction specifies that the amplification mixture comprises both primers at the concentration of no less than 3.0×10⁻⁶M each.

In specific embodiments, the instruction specifies that the sample may be added, as long as the amplification mixture comprises the target nucleic acid of at least than 1.0×10⁻¹³M. In specific embodiments, the instruction species that the sample may be added, as long as the amplification mixture comprises the target nucleic acid of at least than 1.0×10⁻¹⁴ M. In specific embodiments, the instruction species that the sample may be added, as long as the amplification mixture comprises the target nucleic acid of at least than 1.0×10⁻¹³M. In specific embodiments, the instruction species that the sample may be added, as long as the amplification mixture comprises the target nucleic acid of at least than 1.0×10⁻¹⁶M. In specific embodiments, the instruction species that the sample may be added, as long as the amplification mixture comprises the target nucleic acid of at least than 1.0×10⁻¹⁷M. In specific embodiments, the instruction species that the sample may be added, as long as the amplification mixture comprises the target nucleic acid of at least than 1.0×10⁻¹⁸M. In specific embodiments, the instruction species that the sample may be added, as long as the amplification mixture comprises as few as less than 10 copies of the target nucleic acid molecule.

In specific embodiments, the instruction specifies that the amplification mixture comprises at least 0.5% PEG by volume. In specific embodiments, the instruction specifies that the amplification mixture comprises about 0.5%-10% PEG by volume. In specific embodiments, the instruction specifies that the amplification mixture comprises at least about 0.5% PEG by volume. In specific embodiments, the instruction specifies that the amplification mixture comprises at least about 1% PEG by volume. In specific embodiments, the instruction specifies that the amplification mixture comprises at least about 1.5% PEG by volume. In specific embodiments, the instruction specifies that the amplification mixture comprises at least about 2% PEG by volume. In specific embodiments, the instruction specifies that the amplification mixture comprises at least about 2.5% PEG by volume. In specific embodiments, the instruction specifies that the amplification mixture comprises at least about 3% PEG by volume. In specific embodiments, the instruction specifies that the amplification mixture comprises at least about 3.5% PEG by volume. In specific embodiments, the instruction specifies that the amplification mixture comprises at least about 4% PEG by volume. In specific embodiments, the instruction specifies that the amplification mixture comprises at least about 4.5% PEG by volume. In specific embodiments, the instruction specifies that the amplification mixture comprises at least about 5% PEG by volume. In specific embodiments, the instruction specifies that the amplification mixture comprises at least about 10% PEG by volume.

In specific embodiments, the instruction specifies that the amplification mixture comprises SSB of about 1-50 μg/mL. In specific embodiments, the instruction specifies that the amplification mixture comprises SSB of about 1 μg/mL. In specific embodiments, the instruction specifies that the amplification mixture comprises SSB of about 5 μg/mL. In specific embodiments, the instruction specifies that the amplification mixture comprises SSB of about 12.5 μg/mL. In specific embodiments, the instruction specifies that the amplification mixture comprises SSB of about 25 μg/mL. In specific embodiments, the instruction specifies that the amplification mixture comprises SSB of about 50 μg/mL.

In specific embodiments, the instruction specifies that the amplification mixture has a volume of about 1-30 μL. In specific embodiments, the instruction further specifies that the amplification mixture can be loaded onto a microfluidic device for performing the PCR reaction.

In some embodiments, the kit further comprises an instruction for subjecting the amplification mixture under a thermal cycling protocol to perform PCR. In specific embodiments, the thermal cycling protocol comprises a number of thermal cycles, wherein each thermal cycle comprises incubation at a first temperature, and incubation at a second temperature. In specific embodiments, the first temperature is selected from the range of about 68-78° C., and the second temperature is selected from the range of about 55-69° C. In some embodiments, each thermal cycle further comprises a ramp time of less than 10 s. In a specific embodiments, the thermal cycle protocol comprises incubation at the first temperature selected from the range of about 72-76° C. for about 1 s, and incubation at the second temperature selected from the range of about 61-65° C. for about 1 s, and the total ramp time of less than 2 s, and wherein the total reaction time is less than 8 minutes.

### 5. EXAMPLES

Examples related to the present invention are described below. In most cases, alternative techniques can be used. The examples are intended to be illustrative and are not limiting or restrictive to the scope of the invention. For example, where reagents of a PCR reaction were prepared following a protocol of a scheme, it is understood that conditions may vary, for example, any of the solvents, reaction times, reagents, temperatures, supplements, work up conditions, or other reaction parameters may be varied. For example, it is understood that PCR amplification of a target sequence can be detected using different methods, and where PCR amplification does not need to be monitored in real time, a fluorescence dye is not needed to be included in the amplification mixture. It is also understood that although the nucleic acid target to be detected in the following examples are derived from microbial organisms, application of the current methods and systems are not limited to such application scenario, but rather can be applied to detect other types of genetic samples, such as genetic materials originated from a mammal. Furthermore, although studies in the examples below used specific designs of kits of parts, it is understood that such specific designs are not the only or the best design. Variations in the reagent types, volumes, concentrations, packaging are also possible. It is to be understood that this present disclosure is not limited to the particular methodology, protocols, and reagents described, as these may vary, depending upon the context they are used by those of skill in the art.

General Methods. Unless specified otherwise, the methods and equipment in the following examples were those conventionally used for similar studies in the relevant field. Unless otherwise specified, the test materials used in the following examples were purchased from biochemical reagent stores or other commercial suppliers. All molecular biology and PCR reactions were conducted using standard plates, vials, and EP tube typically employed when working with biological molecules such as DNA, RNA and proteins. Commercial reagents were used as received.

In the following examples, genomic DNA or RNA samples were extracted using the DNA/RNA Isolation Kit purchased from Tiangen Biochemical Technology (Beijing) Co., Ltd. (Beijing, China, catalog number DP422). The solvent of the isothermal reaction buffer is purified water, and the solutes and concentrations were as follows: 20 mM Tris-HCl, 10 mM KCl, 10 mM (NH₄)₂SO₄, 2 mM MgSO₄, 0.1% Triton X-100; pH 8.8 at 25° C. Results for quantitative studies were based on at least three repeated studies.

### 5.1 Example 1: Optimization of Primer Concentration for the Accelerated Strand Exchange Amplification (SEA) Reaction System

The following studies were performed to test the effect of primer concentration on the amplification speed of the accelerated SEA reaction.

A pair of specific primers were designed by NUPACK software (www.nupack.org/) based on a target nucleic acid sequence selected from the hypervariable region of Listeria monocytogenes 16s rRNA encoding gene. Particularly, the target sequence was synthetic 50-bp fragment having the following sequence:

### 5′-GGGTCATTGGAAACTGGAAGACTGGAGTGCAGAAGAGGAGAGTGGAATTC-3′ (SEQ ID NO:1),

and the primer sequences were:

### Primer 1: 5′-GTCATTGGAAACTGGAAGACTG-3′ (M58822.1 b) (SEQ ID NO:2); and

### Primer 2: 5′-CCACTCTCCTCTTCTGCAC-3′ (M58822.1 b) (SEQ ID NO:3).

The primers and target fragment were commercially synthesized (Sangon Biotech, Shanghai, China). DNA polymerase, dNTPs solutions, other buffer solutions, and fluorescent dyes (e.g. Evagreen) were purchased with the Strand Exchange Amplification (SEA) Detection Kit from Navid Biotechnology Co., Ltd. (Qingdao, China).

Then the synthesized primers and L. monocytogenes genomic materials were mixed with the other PCR reactants to form a 10 μL amplification mixture as shown in Table 1 below. To optimize the primer concentration for the amplification speed, four units of amplification mixtures were made, each containing the polymerase at the final concentration of 0.24 U/μL, and containing the primers at the final concentration of 1.5×10⁻⁶ M, 2.0×10⁻⁶ M, 2.5×10⁻⁶M, and 3.0×10⁻⁶M, respectively. A negative control group (NTC) of amplification mixture having the same contents but replacing the L. monocytogenes genomic materials with water was included.

TABLE 1
Amplification mixture contents for optimization of primer concentration.

| Content | Final Concentration |
|---|---|
| Primer 1 | 1.5 × 10⁻⁶M, 2.0 × 10⁻⁶M, |
| | 2.5 × 10⁻⁶M, or 3.0 × 10⁻⁶M |
| Primer 2 | 1.5 × 10⁻⁶M, 2.0 × 10⁻⁶M, |
| | 2.5 × 10⁻⁶M, or 3.0 × 10⁻⁶M |
| Synthetic Target DNA | 1.0 × 10⁻¹²M |
| dNTPs | 8 mM |
| Isothermal | 1× |
| reaction buffer | |
| Evagreen | 20× |
| ET SSB | 5 μg/mL |
| Bst DNA polymerase | 0.24 U/μL |
| polyethylene glycol 200 | 100% |
| Water | Make up to 10 μL total |
| | volume |

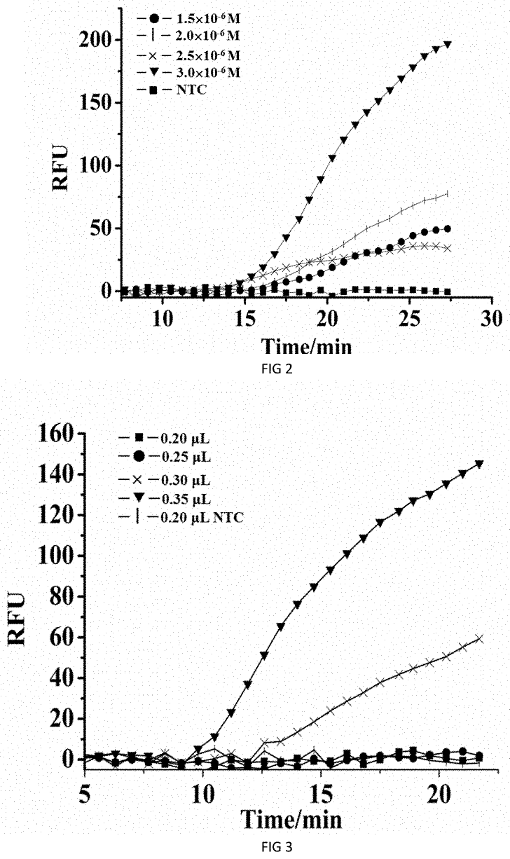

To carry out the accelerated SEA reaction, the amplification mixtures were subjected to swift thermal cycles between 76° C. and 62° C., using the CFX Connect™ RealTime PCR System (Bio-Rad, CA). Particularly, Particularly, each thermal cycle was constituted of incubating the amplification mixture at 76° C. for 1 second (s), before immediately reducing the temperature to 62° C., and incubating the amplification mixture at 62° C. for another 1 s, before immediately increasing the temperature back to 76° C. To monitor the amplification in real time, fluorescent signal emitted from the amplification mixture was scanned every two thermal cycles, and plotted over time in FIG. 2.

As shown in the figure, for each primer concentration tested, the accelerated SEA reaction produced detectable amplification of the target sequence in less than 20 minutes. Particularly, increasing the primer concentration to 3.0×10⁻⁶ M significantly increased the efficiency and speed of amplification, shortening the time needed for detecting the target nucleic acid in the sample to less than about 15 minutes.

### 5.2 Example 2: Optimization of Polymerase Concentration for the Accelerated Strand Exchange Amplification (SEA) Reaction System

The following studies were performed to test the effect of polymerase concentration on the amplification speed of the accelerated SEA reaction.

Particularly, the same primers (SEQ ID NOS:1 and 2) were designed for the same target sequence in Listeria monocytogenes genome (SEQ ID NO:1) as described in Example 1 above. The primers and L. monocytogenes genomic materials were produced as described above, and mixed with the other PCR reactants to form a 10 μL amplification mixture as shown in Table 2 below. To optimize the polymerase concentration for the amplification speed, four units of amplification mixtures were made, each containing the primers at the final concentration of 3×10⁻⁶M, and containing the polymerase at the final concentration of 0.16 U/μL, 0.20 U/μL, 0.24 U/μL, and 0.28 U/μL (corresponding to 0.20 μL, 0.25 μL, 0.30 μL, and 0.35 μL of a 8 U/μL enzyme stock solution), respectively. A negative control group (NTC) of amplification mixture having the same contents but replacing the L. monocytogenes genomic materials with water was included.

TABLE 2
Amplification mixture contents for
optimization of polymerase concentration.

| Content | Final Concentration |
|---|---|
| Primer 1 | 3.0 × 10⁻⁶M |
| Primer 2 | 3.0 × 10⁻⁶M |
| Synthetic Target DNA | 1.0 × 10⁻¹²M |
| dNTPs | 8 mM |
| Isothermal | 1× |
| reaction buffer | |
| Evagreen | 20× |
| ET SSB | 5 μg/mL |
| Bst DNA polymerase | 0.16 U/μL, 0.20 U/μL, |
| | 0.24 U/μL, or 0.28 U/μL |
| Polyethylene glycol | 100% |
| (PEG) 200 | |
| Water | Make up to 10 μL total |
| | volume |

To carry out the accelerated SEA reaction, the amplification mixtures were subjected to swift thermal cycles between 76° C. and 62° C., using the CFX Connect™ RealTime PCR System (Bio-Rad, CA). Particularly, each thermal cycle was constituted of incubating the amplification mixture at 76° C. for 1 s, before immediately reducing the temperature to 62° C., and incubating the amplification mixture at 62° C. for another 1 s, before immediately increasing the temperature back to 76° C. To monitor the amplification in real time, fluorescent signal emitted from the amplification mixture was scanned every two thermal cycles, and plotted over time in FIG. 3.

As shown in the figure, for reactions containing the polymerase at the concentration 0.24 U/μL or 0.28 U/μL, the accelerated SEA reaction produced detectable amplification of the target sequence in less than 20 minutes. Particularly, increasing the polymerase concentration from 0.24 U/μL to 0.28 U/μL further increased the efficiency and speed of amplification significantly, shortening the time needed for detecting the target nucleic acid in the sample from less than about 15 minutes to less than about 10 minutes.

### 5.3 Example 3: Optimization of the Thermal Cycle for the Accelerated Strand Exchange Amplification (SEA) Reaction System

The following studies were performed to test the effect of denaturation temperature on the efficiency and speed of amplification for the accelerated SEA reaction.

Amplification mixture was made as described in Example 1 above, where the primer concentration was kept at 3.0×10⁻⁶M and the polymerase concentration was kept at 0.24 U/μL. The amplification mixture was then subject to different thermal cycles to carry out the PCR reaction, and the effect of the different temperatures on amplification efficiency and speed was evaluated.

Particularly, in each thermal cycle, the amplification mixture was incubated at a higher denaturation temperature for 1 s, which was immediately followed by another 1 s incubation at a lower elongation temperature. The lower elongation temperature can be selected based on the DNA polymerase used for the amplification. In these studies, the elongation temperature was set to 62° C., which was optimal for the Bst DNA polymerase activity. Without being bound by the theory, it was contemplated that slight temperature differences may significantly impact the speed and duration for the opening of denaturation bubbles in a duplex nucleic acid sample, which in turn would affect efficiency and speed for amplification. In these studies, five denaturation temperature of 74° C., 75° C., 76° C., 77° C. and 78° C. were tested and compared. A negative control group (NTC) of amplification mixture having the same contents but replacing the L. monocytogenes genomic materials with water was included.

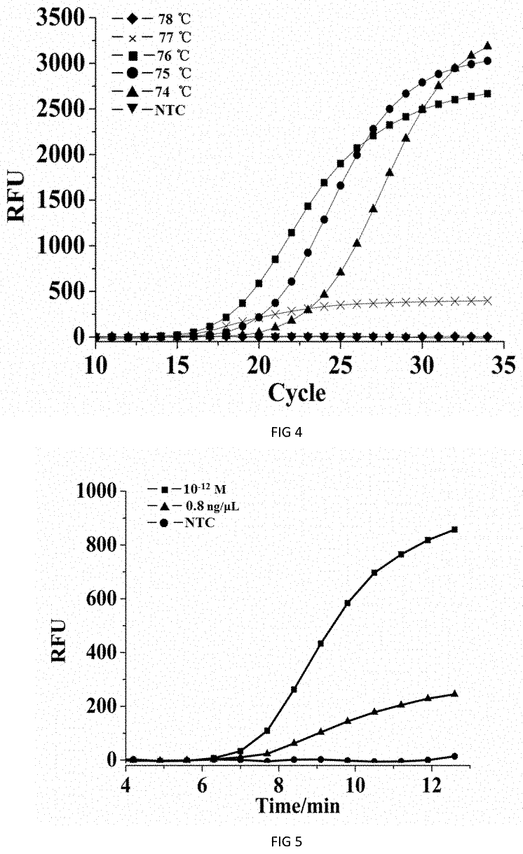

For example, each thermal cycle between 76° C. and 62° C. was constituted of incubating the amplification mixture at 76° C. for 1 s, before immediately reducing the temperature to 62° C., and incubating the amplification mixture at 62° C. for another 1 s, before immediately increasing the temperature back to 76° C. The thermal cycles were repeated for at least 35 cycles for each accelerated SEA reaction. To monitor the amplification in real time, fluorescent signal emitted from the amplification mixture was scanned every two thermal cycles, and plotted over time in FIG. 4.

As shown in the figure, when the denaturation temperature was between 74° C. and 76° C., the accelerated SEA reaction produced detectable amplification of the target sequence in less that about 20 minutes. Particularly, denaturation temperature of 76° C. produced the optimal results among all temperatures tested, resulting in the shortest time needed for detecting the target nucleic acid in the sample.

### 5.4 Example 4: Amplification and Detection of DNA Molecules in a Sample

The following studies were performed to examine the ability of the accelerated SEA method for detecting DNA molecules in a biological sample.

A pair of specific primers were designed by NUPACK software (www.nupack.org/) based on a target nucleic acid sequence. Particularly, the target sequence was

### 5′-GGGTCATTGGAAACTGGAAGACTGGAGTGCAGAAGAGGAGAGTGGAATTC-3′ (SEQ ID NO:1),

and the primer sequences were:

### Primer 1: 5′-GTCATTGGAAACTGGAAGACTG-3′ (M58822.1 b) (SEQ ID NO:2); and

### Primer 2: 5′-CCACTCTCCTCTTCTGCAC-3′ (M58822.1 b) (SEQ ID NO:3).

The primers and target DNA molecules were commercially synthesized (Sangon Biotech, Shanghai, China), and mixed with the other PCR reactants to form a 10 μL amplification mixture as shown in Table 3 below. Particularly, two units of amplification mixtures were made, containing 1.0×10⁻¹² M synthetic target DNA fragments or 0.8 ng/μL L. monocytogenes genomic materials, respectively. The primer concentrations were at 3.0×10⁻⁶M, and polymerase concentration was 0.24 U/μL, and A negative control group (NTC) of amplification mixture having the same contents but replacing the target DNA with water was included.

TABLE 3
Amplification mixture contents for DNA amplification.

| Content | Final Concentration |
|---|---|
| Primer 1 | 3.0 × 10⁻⁶M |
| Primer 2 | 3.0 × 10⁻⁶M |
| Target DNA | 1.0 × 10⁻¹²M synthetic DNA |
| | fragment |
| | OR |
| | 0.8 ng/μL isolated L. |
| | monocytogenes genomic materials |
| dNTPs | 8 mM |
| Isothermal | 1× |
| reaction buffer | |
| Evagreen | 20× |
| ET SSB | 5 μg/mL |
| Bst polymerase | 0.24 U/μL |
| polyethylene glycol 200 | 100% |
| Water | Make up to 10 μL total volume |

To carry out the accelerated SEA reaction, the amplification mixtures were subjected to swift thermal cycles between 76° C. and 62° C., using the CFX Connect™ RealTime PCR System (Bio-Rad, CA). Particularly, Particularly, each thermal cycle was constituted of incubating the amplification mixture at 76° C. for 1 second (s), before immediately reducing the temperature to 62° C., and incubating the amplification mixture at 62° C. for another 1 s, before immediately increasing the temperature back to 76° C. To monitor the amplification in real time, fluorescent signal emitted from the amplification mixture was scanned every two thermal cycles, and plotted over time in FIG. 5.

As shown in the figure, the accelerated SEA method was able to efficiently detect both synthetic DNA fragments and genomic nucleic acids of L. monocytogenes within less than 10 minutes at the provided target concentrations, which enables the use of the present methods and kits for point-of-care diagnosis of pathogenic infections.

### 5.5 Example 5: Amplification and Detection of RNA Molecules in a Sample

The following studies were performed to examine the ability of the accelerated SEA method for detecting RNA molecules in a biological sample.

Particularly, the same primers (SEQ ID NOS:2 and 3) were designed as described above for the target RNA sequence having the following sequence

### 5′-GGGTCAUUGGAAACUGGAAGACUGGAGUGCAGAAGAGGAGAGUGGAAUUC-3′ (SEQ ID NO:7)

The primers and synthetic RNA target molecules were produced as described above, and mixed with the other PCR reactants to form a 10 μL amplification mixture as shown in Table 3 below. Particularly, three duplicates of amplification mixtures were made, each containing the primers at the final concentration of 3.0×10⁻⁶M, polymerase at the final concentration of 0.24 U/μL, and target RNA molecules at the concentration of 1.0×10⁻¹²M. A negative control group (NTC) of amplification mixture having the same contents but replacing the target RNA with water was included.

TABLE 4
Amplification mixture contents for RNA amplification.

| Content | Final Concentration |
|---|---|
| Primer 1 | 3.0 × 10⁻⁶M |
| Primer 2 | 3.0 × 10⁻⁶M |
| Synthetic Target RNA | 1.0 × 10⁻¹²M |
| dNTPs | 8 mM |
| Isothermal | 1× |
| reaction buffer | |
| Evagreen | 20× |
| ET SSB | 5 μg/mL |
| Bst DNA polymerase | 0.24 U/μL |
| Polyethylene glycol | 100% |
| (PEG) 200 | |
| Water | Make up to 10 μL total volume |

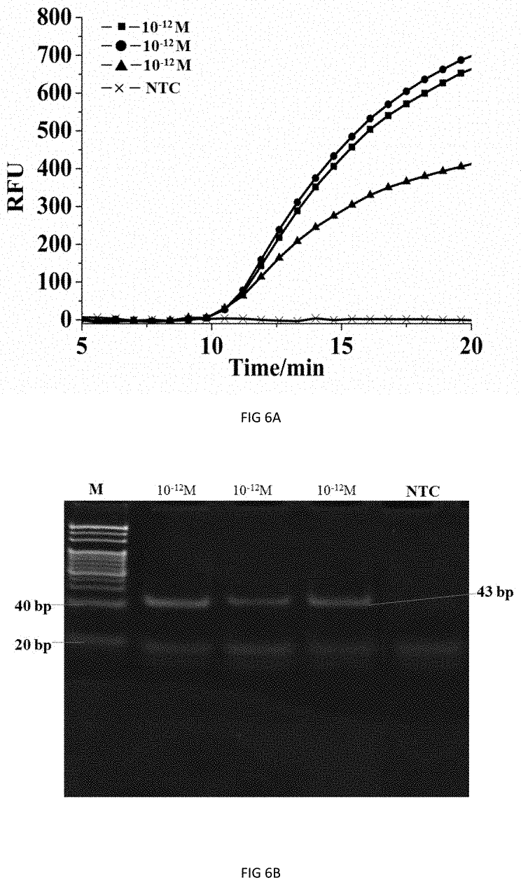

To carry out the accelerated SEA reaction, the amplification mixtures were subjected to swift thermal cycles between 76° C. and 62° C., using the CFX Connect™ RealTime PCR System (Bio-Rad, CA). Particularly, each thermal cycle was constituted of incubating the amplification mixture at 76° C. for 1 s, before immediately reducing the temperature to 62° C., and incubating the amplification mixture at 62° C. for another 1 s, before immediately increasing the temperature back to 76° C. To monitor the amplification in real time, fluorescent signal emitted from the amplification mixture was scanned every two thermal cycles, and plotted over time in FIG. 6A.

As shown in the figure, using the Bst DNA polymerase having reverse transcriptase activity, the accelerated SEA method was able to efficiently detect RNA molecules within about 10 minutes at the provided target concentration. In the three repeated reactions, the amplification arrived at the exponential phase around the same time, while amplification in the negative control group remained undetectable, indicating the method and reaction system is highly reproducible and stable.

Finally, to verify that the observed increase in the fluorescence signal corresponded to specific amplification of the target RNA molecule, after the reactions were completed, the amplification mixtures were loaded onto a 12.5% a polyacrylamide gel for electrophoresis to examine the size of the amplicon. FIG. 6B is a photo of the PAGE gel that shows the specific amplicon having the expected size of 43 bp in the three duplicates of accelerated SEA reactions having the original target concentration at 1.0×10⁻¹² M, and the lack of the specific target band in the negative control (NTC). Lane M was loaded with DNA molecular-weight size markers (DNA ladder), and the bands corresponding to 20 bp and 40 bp DNA fragments are indicated on the figure.

### 5.6 Example 6: Comparison of Isothermal SEA Reactions Under a Constant Temperature and Accelerated SEA Reactions Under Swift Thermal Cycles

The following studies were performed to compare isothermal SEA reactions performed under a constant temperature (such as the procedure described in CN 109136337A) and accelerated SEA reactions under the current swift thermal cycles.

Particularly, the same primers (SEQ ID NOS:1 and 2) were designed as described above for the same target sequence in Listeria monocytogenes genome (SEQ ID NO:1). The primers and L. monocytogenes genomic materials were produced as described above, and mixed with the other PCR reactants to form a 10 μL amplification mixture as shown in Table 5 below. Particularly, to examine and compare speed and sensitivity of the two methods in amplifying and detecting a trace amount of target nucleic acid present in a sample, amplification mixtures containing a 50-bp synthetic fragment of L. monocytogenes genomic sequence at a series of initial concentrations (1.0×10⁻¹¹ M, 1.0×10⁻¹² M, 1.0×10⁻¹³ M, 1.0×10⁻¹⁴ M, 1.0×10⁻¹⁵ M, 1.0×10⁻¹⁶M, 1.0×10⁻¹⁷ M, or 1.0×10⁻¹⁸ M) were made and compared. A negative control group (NTC) of amplification mixture having the same contents but replacing the target with water was included.

TABLE 5
Amplification mixture contents for comparing detection sensitivities.

| | Final Concentration | Final Concentration |
|---|---|---|
| Content | (Accelerated SEA) | (Isothermal SEA) |
| Primer 1 | 3.0 × 10⁻⁶M | 1.5 × 10⁻⁶M |
| Primer 2 | 3.0 × 10⁻⁶M | 1.5 × 10⁻⁶M |
| Synthetic | 1.0 × 10⁻¹¹M, 1.0 × 10⁻¹²M, | 1.0 × 10⁻¹¹M, 1.0 × 10⁻¹²M, |
| Target DNA | 1.0 × 10⁻¹³M, 1.0 × 10⁻¹⁴M, | 1.0 × 10⁻¹³M, 1.0 × 10⁻¹⁴M, |
| | 1.0 × 10⁻¹⁵M, 1.0 × 10⁻¹⁶M, | 1.0 × 10⁻¹⁵M, 1.0 × 10⁻¹⁶M, |
| | 1.0 × 10⁻¹⁷M, or | 1.0 × 10⁻¹⁷M, or |
| | 1.0 × 10⁻¹⁸M | 1.0 × 10⁻¹⁸M |
| dNTPs | 8 mM | 8 mM |
| Isothermal | 1× | 1× |
| reaction | | |
| buffer | | |
| Evagreen | 20× | 20× |
| ET SSB | 5 μg/mL | 5 μg/mL |
| Bst DNA | 0.24 U/μL | 0.16 U/μL |
| polymerase | | |
| polyethylene | 100% | 100% |
| glycol 200 | | |
| Water | Make up to total of 10 μL | Make up to total of 10 μL |

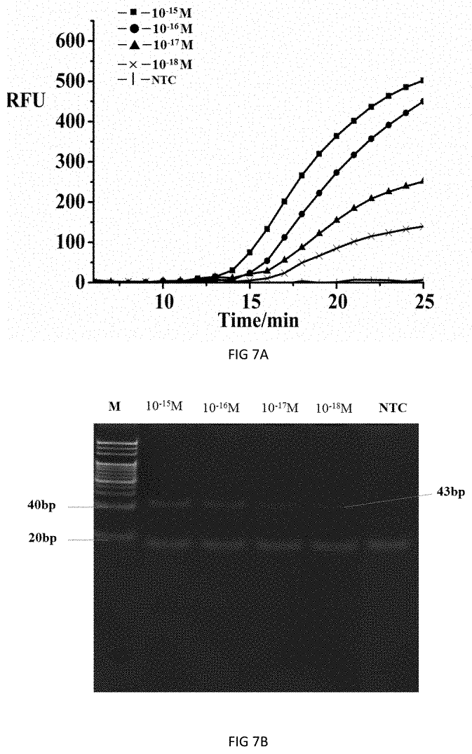

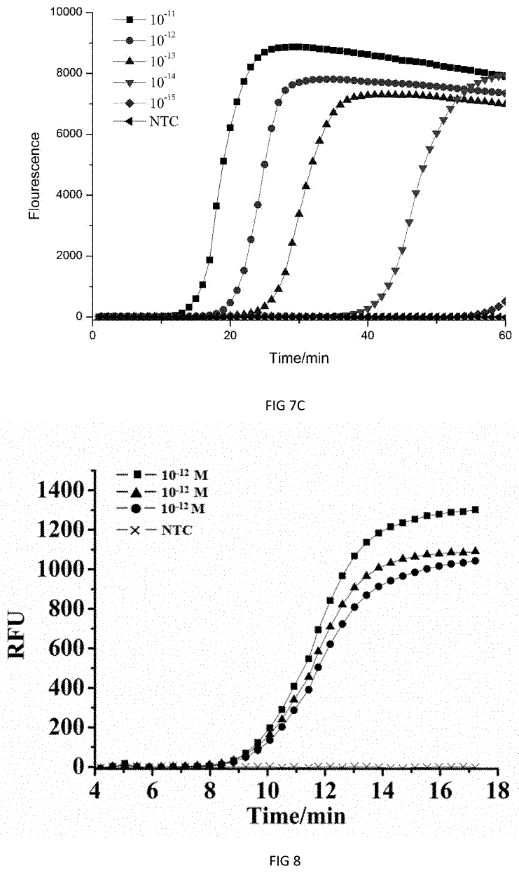

As described above in Example 1, to perform the accelerated SEA reaction, the amplification mixture was subjected to swift thermal cycles between 76° C. and 62° C. using the CFX Connect™ RealTime PCR System (Bio-Rad, CA). Particularly, each thermal cycle was constituted of incubating the amplification mixture at 76° C. for 1 s, before immediately reducing the temperature to 62° C., and incubating the amplification mixture at 62° C. for another 1 s, before immediately increasing the temperature back to 76° C. To monitor the amplification in real time, fluorescent signal emitted from the amplification mixture was scanned every two thermal cycles, and plotted over time in FIG. 7A (data not shown for 1.0×10⁻¹¹ M, 1.0×10⁻¹² M, 1.0×10⁻¹³ M, and 1.0×10⁻¹⁴ M samples).

Further, to verify that the observed increase in the fluorescence signal corresponded to specific amplification, after the reactions were completed, the amplification mixtures were loaded onto a 12.5% a polyacrylamide gel for electrophoresis to examine the size of the amplicon. FIG. 7B is a photo of the PAGE gel that shows the specific amplicon having the expected size of 43 bp in the accelerated SEA reactions having original target concentration at 1.0×10⁻¹⁵ M, 1.0×10⁻¹⁶ M, 1.0×10⁻¹⁷ M, and 1.0×10⁻¹⁸ M, and the lack of the specific target band in the negative control (NTC). Lane M was loaded with DNA molecular-weight size markers (DNA ladder), and the bands corresponding to 20 bp and 40 bp DNA fragments are indicated on the figure.

To perform the isothermal SEA reaction under a constant temperature) the amplification mixture was incubated at 62° C. using the CFX Connect™ RealTime PCR System (Bio-Rad, CA). To monitor the amplification in real time, fluorescent signal emitted from the amplification mixture was scanned at 1-minute intervals, and plotted over time in FIG. 7C (data not shown for 1.0×10⁻¹⁶M, 1.0×10⁻¹⁷ M, and 1.0×10⁻¹⁸ M samples).

As shown in FIGS. 7A and 7C, fluorescence signals of both methods showed good correlation with the increase of initial target concentration in the amplification mixture. That is, the more targets present in the initial sample, the less time it took the method to produce detectable amplification of the target molecule. Notably, the accelerated SEA method (under the swift thermal cycle) performed significantly better than the isothermal SEA method (under the constant temperature) in terms of both speed and sensitivity.

Particularly, as shown in FIG. 7C, at 1.0×10⁻¹⁵ M target concentration, it took about 1 hour for the isothermal SEA method to produce detectable amplification, while as shown in FIG. 7A, it only took the accelerated SEA method less than 15 minutes to produce detectable amplification for all target concentration tested. Hence, the accelerated SEA method reduced the time for detection for about 75% comparing to the isothermal SEA method, shortening the time for detection to as little as 15 minutes.

Furthermore, as shown in FIG. 7C, at 20-minute reaction time, the isothermal SEA method was able to detect target molecules present in the sample at the 1.0×10⁻¹² M or higher concentration, while as shown in FIG. 7A, the accelerated SEA method was able to detect target molecules at as little as 1.0×10⁻¹⁸ M concentration (representing only a few copies of the target nucleic acid in the sample). Hence, for a 15 to 20-minute reaction, the accelerated SEA method increased the sensitivity of detection for at least 1.0×10⁶ folds.

### 5.7 Example 7: Primer Design

Primers were designed and evaluated using NUPACK web tool (www.nupack.org), DNAMelt Web (http://unafold.ma.albany.edu/?q=DINAMelt), NOVOPRO www.novopro.cn/tools/rev_comp.html), and the BLAST algorithm at the NCBI website (www.ncbi.nlm.nih.gov/tools/primer-blast).

DNA primers were synthesized by Personal Biotechnology Co, Ltd. (Shanghai, China). SEA detection kit was purchased from Navid Biotechnology Co, Ltd. (Qingdao, China). DNA extraction kit was purchased from TIANGEN Biotech. Co, Ltd (Beijing, China). Other reagents and buffers were of analytical grade.

Traditional PCR Reaction. Genomic DNA of M. pneumoniae, C. trachoma, S. domestica, B. cereus and S. aureus was extracted by using TIANamp DNA extraction kit (TIANGEN Biotech. Co, Ltd, Beijing, China) according to the manufacture's instruction. Real-time PCR was performed using a CFX Connect™ Real-Time PCR System (Bio-Rad, CA, USA). Reaction mixture of total volume 50 μL containing 20 ng genomic DNA template, 1 μL forward primer and backward primer (10 μNI), 1.5 μL dNTPs (2.5 mM), 0.25 μL Taq polymerase and 5 μEKE standard Taq reaction buffer. The reaction procedure included denaturation at 94° C. for 5 mM, 35 cycles of 94° C. for 30 s, 60° C. for 30 s, 72° C. for 90 s for amplification and final extension at 72° C. for 10 min.

SEA Reaction. SEA reaction was performed in a 10 μL system containing 1 μL template, 1.5 μL each primer (P1 and P2) (10 μNI), 5 μL 2× reaction mix, and 0.25× Eva Green. In order to exclude the influence of the purity of the extracted genome DNA, PCR products (1 pM) were used as the template to carry out the experiment unless stated otherwise. The reaction mixture was incubated at a constant temperature of 57° C., 59° C., 61° C., 63° C. and 65° C., respectively, for 60 min, and SEA amplifications were monitored by CFX Connect™ Real-Time PCR System (Bio-Rad, CA, USA) at 1-min intervals. Additionally, a negative control (NTC) that did not contain any template were also included in each run.

5.7.1 Optimization of Primer Tₘ Values in Relation to Reaction Temperature.

The following example provides an exemplary procedure for selecting the optimal reaction temperature for a given polymerase of choice, as well as primers suitable for the reaction.

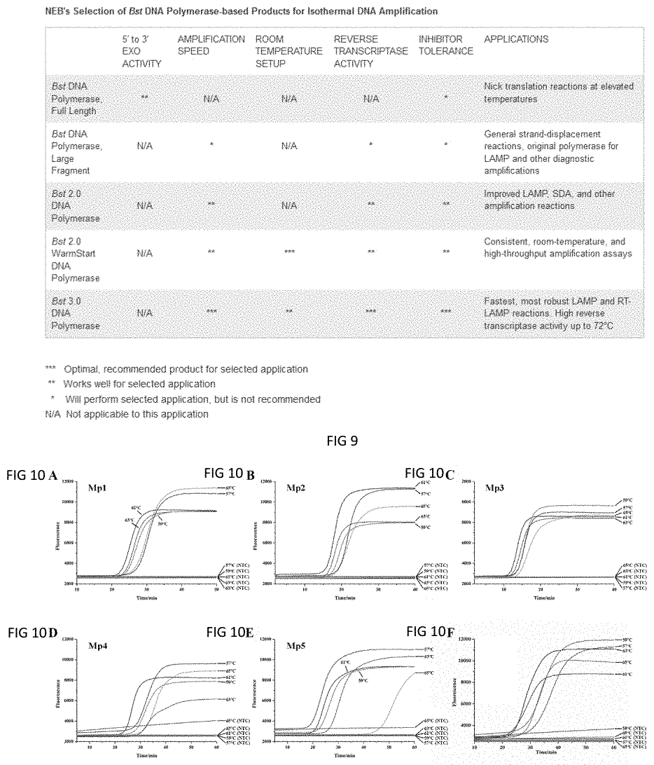

Particularly, a series of primers (Mp1-Mp5) specific to a fragment of M. pneumoniae 16S rRNA sequence having a variety of Tₘ values (about 65° C., 63° C., 61° C., 59° C., or 57° C.) (Table 6) were synthesized. A series of SEA reactions were performed at different constant temperatures at a 2° C. increment over the range of 57° C. to 65° C. at a 2° C. increment (i.e., 65° C., 63° C., 61° C., 59° C., or 57° C.) using Bst 2.0 WarmStart DNA polymerase. To monitor the amplification in real time, fluorescent signal emitted from the amplification mixture was scanned at 1 s intervals, and plotted over time in FIG. 10.

TABLE 6
Primers specific to M. pneumoniae* 16S rRNA

| | | | | Optimal | |
|---|---|---|---|---|---|
| | | | | reaction | |
| Name | | Sequences | | temperature | Tt |
| (SEQ ID NO:) | | (5′ to 3′) | Tm (° C.) | (° C.) | (min) |
| M. pneumoniae | | | | | |
| Mp1 | P1(8) | TCGCGGTAATACATAGGTCGC | 65.9 | 61 | 22 |
| | P2(9) | GCCCAATAAATCCGGATAACGC | 65.3 | | |
| Mp2 | P1(10) | GTCTGGTGTTAAAGGCAGC | 62.1 | 61 | 15 |
| | P2(11) | TCCAATGCATACAACTGTTAAGC | 63.0 | | |
| Mp3 | P1(12) | GCAAGGGTTCGTTATTTGATG | 61.3 | 61 | 11 |
| | P2(13) | CTAGCTGATATGGCGCAC | 61.8 | | |
| Mp4 | P1(14) | GCTATGCTGAGAAGTAGAATAG | 58.8 | 61 | 23 |
| | P2(15) | GTGTCTCAGTCCCATTGT | 59.7 | | |
| Mp5 | P1(16) | AATGACTTTAGCAGGTAATG | 57.4 | 61 | 20 |
| | P2(17) | TGGTACAGTCAAACTCTA | 56.4 | | |

*GenBank accession number is CP017343.1

As shown in FIG. 10, the shortest time for a reaction to produce detectable amplification (threshold time (Tt)) for the primer pairs Mp1-Mp5 were 22 min, 15 min, 11 min, 23 min and 20 min, respectively. Further, among the five reaction temperatures tested, the shortest Tt was achieved at the reaction temperature of 61° C., 61° C., 61° C., 61° C., and 57° C. for primer pairs Mp1-Mp5, respectively. The observed results were summarized in Table 1 above.

These results demonstrated that when using Bst DNA polymerase for the SEA or accelerated SEA reactions, reaction temperature of about 61° C., and primers having Tₘ of about 61° C. can be beneficially selected and used.

Subsequently, to demonstrate that the optimal conditions as determined by the above procedure (e.g., reaction temperature and primer characteristics) are applicable to real-world application scenarios, optimal conditions as determined above were applied to reactions using M. pneumoniae genomic DNA as the target (as opposed to synthetic and/or purified DNA fragments as in a research lab setting). Particularly, 40 ng of M. pneumoniae genome DNA was utilized as the template for SEA reaction with the primer pair Mp3 under the same series of reaction temperatures (i.e., 65° C., 63° C., 61° C., 59° C., or 57° C.). Similarly results were observed: the reaction using primer pair Mp3 carried out at 61° C. exhibited the shortest Tt value. Although the shortest Tt value (around 20 min) in this study was longer than the above studies using amplified DNA fragments as the target, this difference can be attributed to genomic nucleic acids are less likely to form denaturation bubbles at target sites than target DNA fragments (FIG. 10F). These results further demonstrate that the procedure and protocols exemplified herein can be used to determine optimal reaction temperature and primer Tₘ value for the SEA methods and present accelerated SEA methods.

During the above study for optimization of primer Tₘ value and reaction temperature, it was observed that the Tt value was also related to the difference between two primers' Tₘ values in a primer pair. The following example provides further exemplary procedures for selecting primers having beneficial Tₘ characteristics.

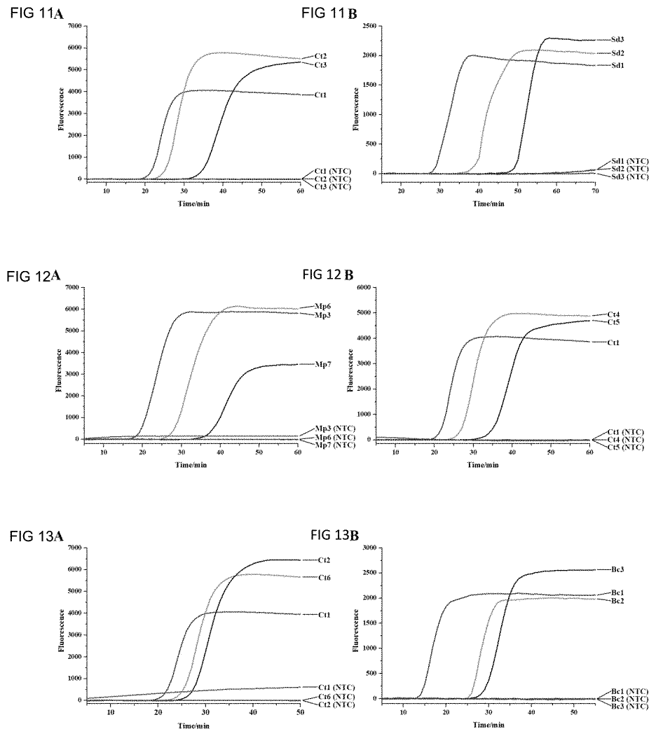

Particularly, primer pairs specific to C. trachoma (Ct1-Ct3) or S. domestica (Sd1-Sd3) having distinct Tₘ value differences were designed and employed in SEA reactions executed at 61° C. (Table 7). The average Tₘ values of the primer pairs were all closed to 61° C. to exclude the possible effect of this factor. As shown in FIG. 11, the primer pairs with smallest Tₘ value difference exhibited the shortest Tt value, while those with largest difference showed the highest Tt value, whether for the primers specific to C. trachoma or S. domestica. It is contemplated that the primers with similar Tₘ values generally have similar annealing temperatures, thus the amplification reactions induced by the primers have similar rate, in which case the SEA reactions were more likely to acquired higher efficiency (Thornton et al., “Real-time PCR (qPCR) primer design using free online software,” Biochem. Mol. Biol. Edu., (2011) 39: 145-154). These results demonstrated that a pair primers having similar Tₘ value can be beneficially selected for the SEA reaction and the accelerated SEA reaction.

TABLE 7
SEA primers specific to C. trachoma* 16S rRNA and S. domestica** 18S rRNA

| | | | | |
|---|---|---|---|---|
| | | | Tm | |
| Name | Sequences | Tm | difference | Tt |
| (SEQ ID NO:) | (5′ to 3′) | (° C.) | (° C.) | (min) |

C. trachoma

| | | | | | |
|---|---|---|---|---|---|
| Ct1 | P1(18) | GTCTAGGCGGATTGAGAGATTGG | 60.9 | 0.0 | 21 |
| | P2(19) | GTGTCTCAGTCCCAGTGTTGG | 60.9 | | |
| Ct2 | P1(20) | TAGTAATGCATAGATAATTTGTCCTT | 60.0 | 1.7 | 24 |
| | P2(21) | GTTTCCAACCGTTATTCCCAAG | 61.7 | | |
| Ct3 | P1(22) | ATCTTAGGACCTTTCGGTTAAGG | 62.8 | 2.5 | 33 |
| | P2(23) | ACTAGCTGATATCACATAGACTC | 60.3 | | |

S. domestica

| | | | | | |
|---|---|---|---|---|---|
| Sd1 | P1(24) | CCACCAAACACATGCATAC | 62.4 | 0.3 | 29 |
| | P2(25) | CATGGGCTTGGGTTTACTATG | 62.1 | | |
| Sd2 | P1(26) | AGCACTATCCATCACCATTG | 60.2 | 1.1 | 38 |
| | P2(27) | AGGGTGAAGTATACGCCTAG | 61.3 | | |
| Sd3 | P1(28) | GACCCACCAAACACATGCA | 62.1 | 1.2 | 49 |
| | P2(29) | GGGCTTGGGTTTACTATGTGG | 60.9 | | |

*GenBank accession number is NR_025888.1
**GenBank accession number is JN601073.1

5.7.2 Optimization of Primer 3′ G/C Content.

The following example provides an exemplary procedure for optimizing the G/C content of a primer to be used in connection with the present method.

Particularly, SEA reactions were performed using different primer pairs specific to a target sequence in M. pneumoniae 16s rRNA (Mp3, Mp6 and Mp7) or a target sequence in C. trachoma 16S rRNA (Ct1, Ct4 and Ct5). The polymerase selected for this study was a Bst DNA polymerase. Particularly, the M. pneumoniae specific primers were designed such that the total number of G and C in a 5-nt region at the 3′ end ranged from 1 to 4; while the C. trachoma specific primers were designed such that the total number of G and C in the 5-nt region at the 3′end were either 2 or 3. Additionally, the primers were also designed to have similar Tm values near 61° C., and the reactions were carried out at the constant temperature of 61° C. To monitor the amplification in real time, fluorescent signal emitted from the amplification mixture was scanned at 1 s intervals, and plotted over time in FIG. 10. The total number of 3′-terminal G/C in a particular primer pair, each primer's G/C contents in a 5-nt region at the primer's 3′end, and reactions' Tt values were recorded in Table 8 below.

TABLE 8
SEA primers specific to M. pneumoniae* and C. trachoma** 16S rRNA

| | | | | | |
|---|---|---|---|---|---|
| | | | Number of | Number | |
| | | | G/C in 3′ end | of 3′- | |
| Name | Sequences | Tm | last five | terminal | Tt |
| (SEQ ID NO:) | (5′ to 3′) | (° C.) | nucleotides | G/C | (min) |

M. pneumoniae

| | | | | | | |
|---|---|---|---|---|---|---|
| Mp3 | P1 (30) | GCAAGGGTTCGTTATTTGATG | 60.9 | 2 | 2 | 18 |
| | P2 (31) | CTAGCTGATATGGCGCAC | 60.9 | 4 | | |
| Mp6 | P1 (32) | GACATCCTTGGCAAAGTT | 60.0 | 1 | 1 | 27 |
| | P2 (33) | CGGTTAACCTCCATTATGTTTC | 61.7 | 2 | | |
| Mp7 | P1 (34) | CGCATAAGAACTTTGGTTCG | 62.8 | 3 | 1 | 36 |
| | P2 (35) | GCAGGTCCTTTCAACTTTGATTCA | 60.3 | 1 | | |

C. trachoma

| | | | | | | |
|---|---|---|---|---|---|---|
| Ct1 | P1 (36) | GTCTAGGCGGATTGAGAGATTGG | 62.4 | 2 | 2 | 21 |
| | P2 (37) | GTGTCTCAGTCCCAGTGTTGG | 62.1 | 3 | | |
| Ct4 | P1 (38) | GTAAAGGCCTACCAAGGCT | 60.2 | 3 | 1 | 27 |
| | P2 (39) | CTCTCAATCCGCCTAGACG | 61.3 | 3 | | |
| Ct5 | P1 (40) | CGTTAAAGAAGGGGATCTTAGG | 62.1 | 2 | 1 | 33 |
| | P2 (41) | ATAGACTCTCCCTTAACCGAAA | 60.9 | 2 | | |

*GenBank accession number is CP017343.1
**GenBank accession number is NR_025888.1

As shown in FIG. 11A and Table 8 above, the result of M. pneumoniae specific primers revealed that the primers with higher 3′ end G/C contents near the 3′ end exhibited lower Tt values, suggesting a higher G/C content at the 3′ end of a primer can be beneficially selected. This observation is in contrast to the design of conventional PCR primers, where avoiding a high G/C content at primer's 3′ end was reported to be beneficial (Simonsson et al., “DNA tetraplex formation in the control region of c-myc,” Nucleic Acids Res., (1998) 26:1167-1172).

Furthermore, it was also observed that among the three C. trachoma specific primer pairs having similar G/C contents in the 3′ end region, the primer pair (Ct1) having G or C as 3′-terminal nucleotides in both primers (P1, P2) produced the lowest Tt value. The same phenomenon was also observed for the M. pneumoniae specific primers. These results demonstrated that a relatively more stable hybridization via G/C base pairing between a primer and its target site was beneficial, which would avoid the primer being easily replaced by the original complementary strand. Furthermore, the stable structure formed by the terminal base pair would facilitate the initiation of primer extension by the polymerase, as well as prevent non-specific amplification (Rodríguez-Lázaro et al., “Real-time PCR in food science: introduction,” Curr. Issues Mol. Biol (2013) 15: 25-38).

Accordingly, this study demonstrated that the primers according to the present disclosure can beneficially have at least 2 G and/or C in the 5-nt region at the end where the polymerase imitates primer extension. Furthermore, having G or C as the terminal nucleotide at the end the polymerase initiates primer extension is beneficial.

5.7.3 Optimization Primer Sequences Based on Complementarily.

The following example provides an exemplary procedure for optimizing the primer sequence to avoid or reduce the possibility of forming self-complementary secondary structure within the primer molecule.

Particularly, influence of self-complementary in a primer sequence or and 3′ complementary between a pair of primers was assessed using the SEA method. Particularly, different primer pairs specific to C. trachoma (Ct1, Ct6 and Ct2) or B. cereus (Bc1-Bc3) were analyzed for their levels of potential self-complementarity or 3′ complementarity using the BLAST algorithm available on the NCBI website (www.ncbi.nlm.nih.gov/tools/primer-blast). The primer sequences, and predicted number of complementarity sites were summarized in Table 9 below. The primers were then used to perform SEA reactions under the conditions described above. To monitor the amplification in real time, fluorescent signal emitted from the amplification mixture was scanned at 1 s intervals, and plotted over time in FIG. 13.

TABLE 9
SEA primers specific to C. trachoma* and B. cereus** 16S rRNA

| | | | | | |
|---|---|---|---|---|---|
| | | | Self- | 3′ end | |
| Name | Sequences | Tm | Comple- | Comple- | Tt |
| (SEQ ID NO:) | (5′ to 3′) | (° C.) | mentary | mentary | (min) |

C. trachoma

| | | | | | | |
|---|---|---|---|---|---|---|
| Ct1 | P1 (42) | GTCTAGGCGGATTGAGAGATTGG | 60.9 | 4 | 0 | 21 |
| | P2 (43) | GTGTCTCAGTCCCAGTGTTGG | 60.9 | 3 | 2 | |
| Ct6 | P1 (44) | CTTTCGGTTAAGGGAGAGTC | 61.7 | 6 | 3 | 24 |
| | P2 (45) | CCACCAACTAGCTGATATCAC | 61.1 | 8 | 6 | |
| Ct2 | P1 (46) | TAGTAATGCATAGATAATTTGTCCTT | 60.0 | 6 | 3 | |
| | P2 (47) | GTTTCCAACCGTTATTCCCAAG | 61.7 | 5 | 0 | 27 |

B. cereus

| | | | | | | |
|---|---|---|---|---|---|---|
| Bc1 | P1 (48) | GACTGCCGGTGACAAACC | 66.2 | 4 | 1 | 14 |
| | P2 (49) | CGTCATCCCCACCTTCCT | 64.6 | 2 | 0 | |
| Bc2 | P1 (50) | GTTGCGACAGCTCTAGGAC | 60.0 | 4 | 2 | 26 |
| | P2 (51) | CTGCACCACCGATAATTGC | 59.3 | 4 | 2 | |
| Bc3 | P1 (52) | CGCTAGTAATCGCGGATCAGC | 59.3 | 6 | 6 | 28 |
| | P2 (53) | CGGGAACGTATTCACCGC | 60.9 | 4 | 2 | |

*GenBank accession number is NR_025888.1
**GenBank accession number is NR_152692.1

As shown in FIG. 13, the number of the complementary sites in a primer pair showed a positive correlation with the Tt value of the corresponding reaction, where the primer pair having the smallest total number of potential complementary sites were associated with the lowest Tt value. It was also observed that among the B. cereus specific primers, the primer pair associated with the lowest Tt value (Bc1) had the highest Tₘ (65° C.) among all primers tested. Further, the 3′ terminal nucleotide of the Bc1 P2 primer was neither G nor C. This observation suggested that the negative impact of primer sequence complementarity overweighed the positive influences of primer G/C content or Tₘ value on the overall efficiency and speed of SEA method or accelerated SEA method.

This study thus demonstrated that avoiding or reducing self-complementarity and/or 3′ complementarity in the primer sequence can be beneficial for the present methods.

5.7.4 Priority of Primer Design Considerations (Tₘ Value and 3′ End C/G Content)

During the actual primer design process, different considerations for optimizing the primer sequence can lead to contradicting selections. For example, as shown in FIG. 13 and Table 9, the negative impact of primer sequence complementarity overweighed the positive influences of primer G/C content or Tₘ value on the overall efficiency and speed of the amplification. The following study further provides exemplary process and protocols for determining the order of priority between the selection of a primer's Tₘ value and G/C content at the 3′ end.

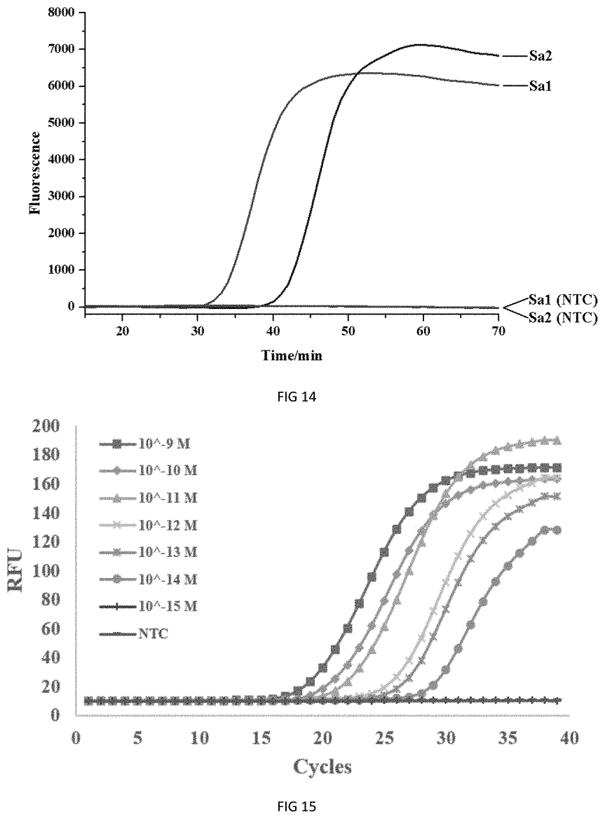

Specifically, two of the primer pairs specific to S. aureus (Sal and Sat) were employed for SEA reactions using 4 ng genomic DNA as template. Particularly, for the Sal primer pair, The Tₘ value and the Tₘ value difference between the two primers were around 65° C. and 2.2° C., respectively; and the 3′ terminal nucleotides for both primers were either G or C. For the Sat primer pair, The Tₘ value and the Tₘ value difference between the two primers were around 61° C. and 1.1° C., respectively; and the 3′ terminal nucleotides for both primers were either A or T. The primer sequences and characteristics were summarized in Table 10 below. The primers were then used to perform SEA reactions under the conditions described above. To monitor the amplification in real time, fluorescent signal emitted from the amplification mixture was scanned at 1 s intervals, and plotted over time in FIG. 14.

TABLE 10
Nuclear acid sequences specific to S. aureus* 16S rRNA

| | | | | Number of | | |
|---|---|---|---|---|---|---|
| | | | | G/C in 3′ | Number of | |
| Name | | Sequences | Tm | end last five | 3′ terminal | Tt |
| (SEQ ID NO:) | | (5′ to 3′) | (° C.) | nucleotides | G/C | (min) |
| S. aureus | | | | | | |
| Sa1 | P1(54) | GGTTCAAAAGTGAAAGACGGTCTTG | 63.9 | 2 | 2 | 37 |
| | P2(55) | GCGCGGATCCATCTATAAGTGAC | 66.1 | 3 | | |
| Sa2 | P1(56) | CGCATGGTTCAAAAGTGAA | 60.4 | 2 | 0 | 32 |
| | P2(57) | AGTGACAGCAAGACCGT | 61.5 | 3 | | |

*GenBank accession number is D83356.1.

As shown in FIG. 14 and Table 10, the amplification efficiency of primer pair Sal was significantly lower (Tt=37 min) than that of primer pair Sat (Tt=32 min). Based on these assays, it can be concluded that the selection of a favorable Tₘ value and Tₘ value difference can be beneficially placed at a higher priority than the selection of a favorable 3′ terminal residue or 3′ end G/C content. The observations can be explained as that a proper relationship between primer's Tₘ and reaction temperature would more efficiently facilitate the formation of a stable primer-target duplex structure as compared to the stability provided by G-C base-pairing (Lim et al., “Design and use of group-specific primers and probes for real-time quantitative PCR,” Frontiers of Environmental Science & Engineering in China, (2011) 5:28-39).

In summary, these studies shows that the order of priority for primer design based on the different considerations is (from high to low priority): (1) avoiding/reducing self-complementarity and/or 3′ complementarity in primer sequences, (2) selecting favorable Tₘ value and/or Tₘ value difference, and (3) selecting a favorable terminal C/G content and/or terminal G/C residue. In other words, when a selection of primer sequence based on a lower-priority consideration contradicts with a selection of primer sequence based on a higher-priority consideration, the selection based on the higher-priority consideration can be beneficially adopted.

### 5.8 Example 8: Kits

An example of using a pre-prepared reagent kits for detecting target nucleic acids using the present accelerated SEA method is provided below.

A kit containing Buffer A and Buffer B having the following contents was prepared.

Buffer A:

Isothermal reaction buffer (10×): 1.75 μL;

dNTPs (10 mM): 2 μL;

Primer 1: 7.5 μL (for a final concentration: 3.0×10⁻⁶ M);

Primer 2: 7.5 μL (for a final concentration: 3.0×10⁻⁶ M);

PEG 200 (100%): 0.625 μL;

Evagreen (20×): 0.625 μL;

Buffer B:

Isothermal reaction buffer (10×): 0.75 μL;

ET SSB (500 μg/mL): 0.25 μL;

DNA polymerase (8 U/μL): 0.75 μL

In this example, the primer pair was designed for detecting Staphylococcus aureus in a sample. Particularly, the primers were designed to amplify a fragment of Staphylococcus aureus 16S rRNA encoding gene of having the following sequence:

### 5′-GGTTCAAAAGTGAAAGACGGTCTTGCTGTCACTTATAGATGGATCCGCGC-3′ (SEQ ID NO:4)

and the primer sequences were:

### Primer 1: 5′-GGTTCAAAAGTGAAAGACGGTCTTG-3′ (SEQ ID NO:5); and

### Primer 2: 5′-GCGCGGATCCATCTATAAGTGAC-3′ (SEQ ID NO:6).

Staphylococcus aureus genome was extracted using the DNA/RNA Isolation Kit purchased from Tiangen Biochemical Technology (Beijing) Co., Ltd. (Beijing, China, catalog number DP422) according to manufacture's instruction into a stock solution. Three duplicates were prepared as such: Buffer A and Buffer B were mixed, and 2.5 μL extracted Staphylococcus aureus genomic materials was added to the mixture, and water was added to make up a total volume of 25 μL. A negative control group (NTC) of amplification mixture having the same contents but replacing the Staphylococcus aureus genomic materials with water was included.

To carry out the accelerated SEA reaction. The amplification mixture was subjected to swift thermal cycles between 76° C. and 61° C. using the CFX Connect™ RealTime PCR System (Bio-Rad, CA). Particularly, Particularly, each thermal cycle was constituted of incubating the amplification mixture at 76° C. for 1 second (s), before immediately reducing the temperature to 61° C., and incubating the amplification mixture at 61° C. for another 1 s, before immediately increasing the temperature back to 76° C. To monitor the amplification in real time, fluorescent signal emitted from the amplification mixture was scanned every two thermal cycles, and plotted over time in FIG. 8. As shown in the figure, the three duplicates produced consistent results in terms of amplification while the negative control did not produce detectable fluorescent signal. These results indicate that using the kit produced reproducible and stable results, and reagents separately stored as Buffer A and Buffer B remained stable, and became reactive after mixing together.

### 5.9 Example 9: Performing Accelerated SEA Using a Microfluidic Device

An example for detecting target nucleic acids using the present accelerated SEA method in microfluidic chip is provided below.

A 10 μL reaction mixture containing the following contents was prepared:

Purified water: 0.35 μL
Isothermal reaction buffer (10×): 1 μL
Primer 1: 3 μL (for a final concentration: 3.0×10⁻⁶ M);
Primer 2: 3 μL (for a final concentration: 3.0×10⁻⁶ M);

### PEG 200 (100%): 0.25 μL;

### Evagreen (20×): 0.25 μL;

dUTPs (10 mM):0.8 μL;

### Uracil-DNA Glycosylase (1 U/μL): 0.1 μL;

DNA polymerase (8 U/μL):0.25 μL, and,
Target nucleic acid: 1 μL.

In this example, the primer pair was designed for detecting Staphylococcus aureus in a sample. Particularly, the primers were designed to amplify a fragment of Staphylococcus aureus 16s rRNA encoding gene of having the following sequence:

### 5′-GGTTCAAAAGTGAAAGACGGTCTTGCTGTCACTTATAGATGGATCCGCGC-3′ (SEQ ID NO:4)

and the primer sequences were:

### Primer 1: 5′-GGTTCAAAAGTGAAAGACGGTCTTG-3′ (SEQ ID NO:5); and

### Primer 2: 5′-GCGCGGATCCATCTATAAGTGAC-3′ (SEQ ID NO:6).

Staphylococcus aureus genome was extracted using the DNA/RNA Isolation Kit purchased from Tiangen Biochemical Technology (Beijing) Co., Ltd. (Beijing, China, catalog number DP422) according to manufacturer's instruction into a stock solution. All the regents were mixed and 1.0 μL of different concentration of extracted Staphylococcus aureus genomic materials was added to the mixture to make up a total volume of 10 μL, the concentration of the genomic materials were 1.0×10⁻⁹ M, 1.0×10⁻¹⁰ M, 1.0×10⁻¹¹ M, 1.0×10⁻¹² M, 1.0×10⁻¹³ M, 1.0×10⁻¹⁴ M and 1.0×10⁻¹⁶ M, respectively. A negative control group (NTC) of amplification mixture having the same contents but replacing the Staphylococcus aureus genomic materials with water was included.

Well-mixed the reaction mixture, and then absorbed all the mixture by pipette and injected into reaction chamber from the injection port on the microfluidic Rapi:chip™ (Genesystem, South Korea), every reaction chamber have an injection port and an exhaust port. After all sample mixtures and NTC mixture were injected into the reaction chambers, a sealing film was pasted on the microfluidic chip to seal up the injection port and exhaust port. After that the microfluidic chip was subjected to UF-150 GENECHECKER™ Ultra-Fast Real-time PCR System (Genesystem, South Korea) and ready to carry amplification reaction.

The microfluidic chip was subjected to swift thermal cycles between 76° C. and 60° C. after an incubation at 37° C. for 5 min. each thermal cycle was constituted of incubating the amplification mixture at 76° C. for 1 second (s), before immediately reducing the temperature to 60° C., and incubating the amplification mixture at 60° C. for another 1 s, before immediately increasing the temperature back to 76° C. The rate of temperature rising and decreasing was 8° C./s, and every cycle completed within 12 s. To monitor the amplification in real time, fluorescent signal emitted from the amplification mixture was scanned every thermal cycle, and plotted over time in FIG. 15. As shown in the figure, the appearance time of fluorescent signal had a positive correlation with the concentration of the genomic materials, while the negative control did not produce detectable fluorescent signal.

Studies shown in this example demonstrated that the present accelerated SEA method can amplify and detect a trace amount of target molecule present in a sample (as little as 1.0×10⁻¹⁴ M or about 6.0×10⁴ copies in a 10 μL reaction system) in less than 8 minutes (less than 40 cycles).

### 5.10 Example 10: Reduce Contamination Uracil-DNA Glycosylase (UDG)

The following studies demonstrated that adding the uracil-DNA glycosylase in the amplification mixture can reduce carry-over contamination in the amplification.

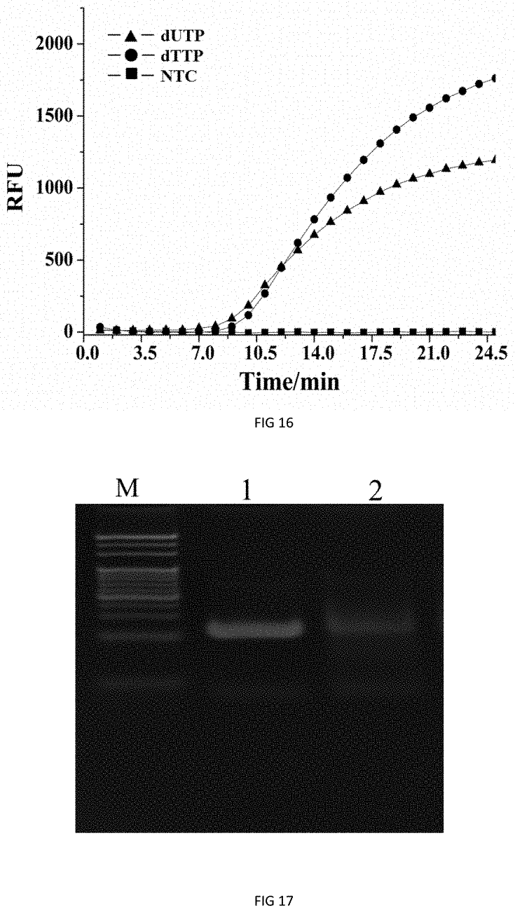

First, following studies were performed to demonstrate that the accelerated SEA can incorporate dUTPs into newly synthesized amplification products. In one reaction (dTTPs; closed circle), the amplification mixture contained dNTPs (dATPs, dGTPs, dTTPs, dCTPs), and in a second reaction (dUTPs; closed triangle), the amplification mixture contained dNTPs (dATPs, dGTPs, dUTPs, dCTPs). A control reaction that contained no target molecule (NTC; closed square) was also included. Accelerated SEA reactions were performed as described above, and fluorescent signal was plotted against time in FIG. 16. As shown, replacing dTTPs with dUTPs did not significantly affect the reaction efficiency, indicating that dUTPs can be used in the accelerated SEA reactions.

Further, the following studies were performed to demonstrate digestion of uracil-containing nucleic acid by UDG. Uracil-containing amplification products from above second reaction which used dUTPs in placed of dTTPs was subjected to UDG digestion. Particularly, for digestion, 10 μL amplification mixture was added with UDG (0.01 U/μL) and incubated at 37° C. for 2 minutes; then the digested product was loaded onto SDS gel for electrophoresis. Another duplicate 10 μL amplification mixture untreated with UDG was loaded onto a separate lane of the SDS gel for comparison. As shown in FIG. 17, after the treatment of UDG, the band in lane 2 was obviously dimmer than untreated band in lane 1, which indicates that UDG degraded the uracil-containing amplification product by cutting the U bases incorporated in the product.

Finally, the following studies were performed to demonstrate that including UDG in the amplification mixture when performing the accelerated SEA can effectively prevent contamination caused by amplification products of prior reactions that exist in the ambient environment, such as floating aerosols.

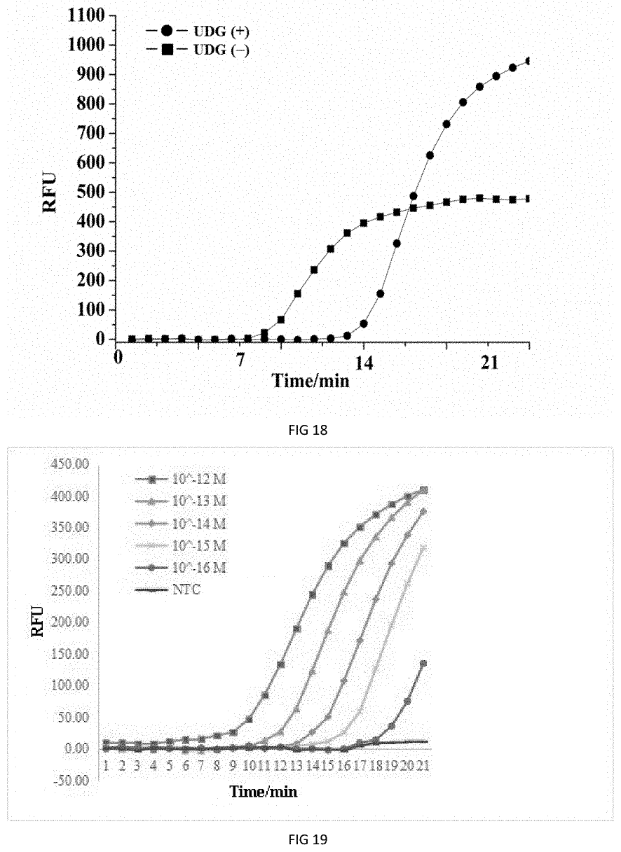

In this study, dATPs, dGTPs, dUTPs, and dCTPs were used for a first round of accelerated SEA reactions. Then the amplified products were used as targets for another round of accelerated SEA reactions. Particularly, Accelerated SEA reactions were performed as described above, and fluorescent signal from the second round of reactions was plotted against time in FIG. 18. As can be seen in the figure, the threshold time (Tt) of the amplification reactions containing 0.01 U/μL UDG (closed circle) was delayed by 3.78 min as compared to the amplification reactions without UDG (closed square), indicating that UDG can effectively prevent the contamination by uracil-containing nucleic acid molecules for the present SEA and accelerated SEA methods.

### 5.11 Example 11: Rapid Amplification and Detection of DNA Molecules in a Sample Using a Thermostable Taq DNA Polymerase

The following studies were performed to examine the ability of the accelerated SEA method for detecting DNA molecules in a biological sample.

A pair of specific primers were designed by NUPACK software (www.nupack.org/) based on a target nucleic acid sequence. Particularly, the target sequence was

### 5′-AGATGTTGAAGGATTCAACCAAATCTCCAGAGTTTGTTAAAACCGTTCCAA-3′ (SEQ ID NO: 58),

and the primer sequences were:

### Primer 1: 5′-ATGTTGAAGGATTCAACCAAATC-3′ (SEQ ID NO: 59);

### Primer 2: 5′-GGAACGGTTTTAACAAACTCTG-3′ (SEQ ID NO: 60).

The primers and target DNA molecules were commercially synthesized (Sangon Biotech, Shanghai, China), and mixed with the other PCR reactants to form a 10 μL amplification mixture as shown in Table 11 below. Particularly, amplification mixtures were made, each containing 1.0×10⁻¹² M, 1.0×10⁻¹³ M, 1.0×10⁻¹⁴M, 1.0×10⁻¹⁵ M or 1.0×10⁻¹⁶M Vibrio parahemolyticus genomic materials, respectively. The primer concentrations were at 5.0×10⁻⁷M, and polymerase concentration was 0.05 U/μL, and A negative control group (NTC) of amplification mixture having the same contents but replacing the target DNA with water was included.

TABLE 11
Amplification mixture contents for DNA amplification

| Contents | Final Concentration |
|---|---|
| Primer 1 | 5.0 × 10⁻⁷M |
| Primer 2 | 5.0 × 10⁻⁷M |
| Target DNA | 1.0 × 10⁻¹²M, 1.0 × 10⁻¹³M, |
| | 1.0 × 10⁻¹⁴M, 1.0 × 10⁻¹⁵M or |
| | 1.0 × 10⁻¹⁶M Vibrio Parahemolyticus |
| | genomic sequence |
| dNTPs | 8 mM |
| Taq buffer | 1× |
| Evagreen | 20× |
| Taq DNA polymeras | 0.05 U/μL |
| 100% polyethylene glycol 200 | 0.625 μL |
| Water | Make up to 10 μL total volume |

To carry out the accelerated SEA reaction, the amplification mixtures were subjected to swift thermal cycles between 76° C. and 61° C., using the CFX Connect™ RealTime PCR System (Bio-Rad, CA). Particularly, Particularly, each thermal cycle was constituted of incubating the amplification mixture at 76° C. for 1 second (s), before immediately reducing the temperature to 61° C., and incubating the amplification mixture at 61° C. for another 1 s, before immediately increasing the temperature back to 76° C. To monitor the amplification in real time, fluorescent signal emitted from the amplification mixture was scanned every two thermal cycles, and plotted over time in FIG. 19.

As shown in the figure, the accelerated SEA method was able to efficiently detect genomic nucleic acids of V. Parahemolyticus at the provided target concentrations, which enables the use of the present methods and kits for point-of-care diagnosis of pathogenic infections.

### 5.12 Example 12: Amplification and Detection of DNA Molecules in a Sample Using a Nucleic Acid Prob

The following studies were performed to examine the ability of the accelerated SEA method using a nucleic acid probe for detecting DNA molecules in a biological sample.

Particularly, a pair of specific primers and a probe were designed according to the following target sequence in the human β-actin gene:

### 5′-CAAATGCTTCTAGGCGGACTATGACTTAGTTGCGTTACACCCTTTCTTGACAAAAC CTAACTTGCG-3′ (SEQ ID NO: 61)

And the primer and probe sequences were:

### Primer 1: 5′-CCTGTGTTATCTTGGAGGTC-3′ (SEQ ID NO: 62);

### Primer 2: 5′-FAM-CCCTGAAGGGCTCTCTGG-BHQ-3′ (SEQ ID NO: 63).

### Probe: 5′-ACCAAAAGAGCTAGAACCAC-3′ (SEQ ID NO: 64).

The primers, probe and target DNA molecules were commercially synthesized (Sangon Biotech, Shanghai, China), and mixed with the other PCR reactants to form a 10 μL amplification mixture as shown in Table 12 below. Particularly, an amplification mixture was made, containing genomic materials isolated from human oral epithelial cells as the target DNA. The primer concentration was at 5.0×10⁻⁷ M, the probe concentration was 6.0×10⁻⁷ M and polymerase concentration was 0.05 U/μL. A negative control group (NTC) of amplification mixture having the same contents but replacing the target DNA with water was included.

TABLE 12
Amplification mixture contents for DNA amplification

| Contents | Final Concentration |
|---|---|
| Primer 1 | 5.0 × 10⁻⁷M |
| Primer 2 | 5.0 × 10⁻⁷M |
| Probe | 6.0 × 10⁻⁷M |
| dNTPs | 8 mM |
| Target DNA | 10⁻¹⁴M synthetic DNA fragment |
| Taq buffer | 1× |
| Taq DNA polymerase | 0.05 U/μL |
| 100% Polyethylene glycol 200 | 0.625 μL |
| Water | Make up to 10 μL total volume |

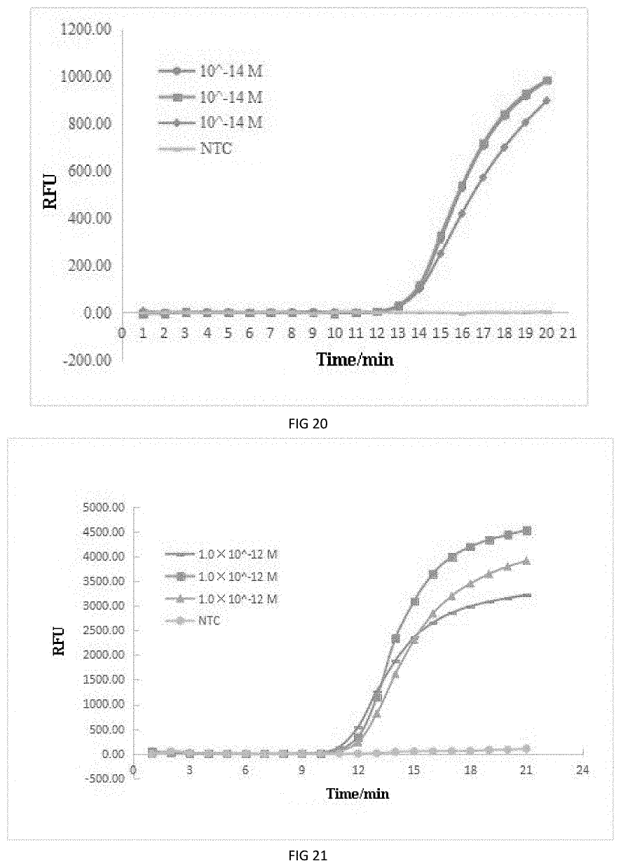

To carry out the accelerated SEA reaction, the amplification mixtures were subjected to swift thermal cycles between 76° C. and 61° C., using the CFX Connect™ RealTime PCR System (Bio-Rad, CA). Particularly, Particularly, each thermal cycle was constituted of incubating the amplification mixture at 76° C. for 1 second (s), before immediately reducing the temperature to 61° C., and incubating the amplification mixture at 61° C. for another 1 s, before immediately increasing the temperature back to 76° C. To monitor the amplification in real time, fluorescent signal emitted from the amplification mixture was scanned every two thermal cycles, and plotted over time in FIG. 20.

As shown in the figure, the accelerated SEA method was able to efficiently detect human β-actin gene from the oral epithelial cells within about 10 minutes at the provided target concentrations. In the three repeated reactions, the amplification arrived at the exponential phase around the same time, while amplification in the negative control group remained undetectable, indicating the method and reaction system is highly reproducible and stable

### 5.13 Example 13: Amplification and Detection of DNA Molecules in a Sample

The following studies were performed to examine the ability of the accelerated SEA method for detecting DNA molecules in a biological sample.

A pair of specific primers were designed by NUPACK software (www.nupack.org/) based on a target nucleic acid sequence. Particularly, the target sequence was

### 5′-AGATGTTGAAGGATTCAACCAAATCTCCAGAGTTTGTTAAAACCGTTCCAA-3′ (SEQ ID NO:58),

and the primer sequences were:

### Primer 1: 5′-ATGTTGAAGGATTCAACCA-3′ (M58822.1 b) (SEQ ID NO:65);

### Primer 2: 5′-GGAACGGTTTTAACAAACT-3′ (M58822.1 b) (SEQ ID NO:66).

The primers and target DNA molecules were commercially synthesized (Sangon Biotech, Shanghai, China), and mixed with the other PCR reactants to form a 10 μL amplification mixture as shown in Table 13 below. Particularly, an amplification mixture was made, containing 1.0×10⁻¹²M synthetic target DNA fragments of L. monocytogenes. The primer concentrations were at 3.0×10⁻⁶M, and polymerase concentration was 0.24 U/μL, and A negative control group (NTC) of amplification mixture having the same contents but replacing the target DNA with water was included.

TABLE 13
Amplification mixture contents for DNA amplification

| Content | Final Concentration |
|---|---|
| Primer 1 | 3.0 × 10⁻⁶M |
| Primer 2 | 3.0 × 10⁻⁶M |
| Target DNA | 1.0 × 10⁻¹²M synthetic DNA fragment |
| dNTPs | 8 mM |
| Isothermal | 1× |
| reaction buffer | |
| 20× Evagreen | 0.625 μL |
| ETSSB | 5 μg/mL |
| Bst DNA polymerase | 0.24 U/μL |
| 100% polyethylene glycol 200 | 0.625 μL |
| Water | Make up to 10 μL total volume |

To carry out the accelerated SEA reaction, the amplification mixtures were subjected to swift thermal cycles between 76° C. and 55° C., using the CFX Connect™ RealTime PCR System (Bio-Rad, CA). Particularly, Particularly, each thermal cycle was constituted of incubating the amplification mixture at 76° C. for 1 second (s), before immediately reducing the temperature to 55° C., and incubating the amplification mixture at 55° C. for 3 s, before immediately increasing the temperature back to 76° C. To monitor the amplification in real time, fluorescent signal emitted from the amplification mixture was scanned every two thermal cycles, and plotted over time in FIG. 21.

As shown in the figure, the accelerated SEA method was able to efficiently detect synthetic DNA fragments of L. monocytogenes within less than 10 minutes at the provided target concentrations, which enables the use of the present methods and kits for point-of-care diagnosis of pathogenic infections.

### 5.14 Example 14: Amplification and Detection of microRNA Molecules in a Sample

The following studies were performed to examine the ability of the accelerated SEA method for detecting microRNA molecules in a biological sample.

A pair of specific primers were designed by NUPACK software (www.nupack.org/) based on a target nucleic acid sequence. Particularly, the target sequence was

### 5′-GCUUAUCAGACUGAUGUUGA-3′ (SEQ ID NO:67),

and the primer sequences were:

### Primer 1: 5′-GCTTATCAGA-3′ (M58822.1 b) (SEQ ID NO:68);

### Primer 2: 5′-TCAACATCAG-3′ (M58822.1 b) (SEQ ID NO:69).

The primers and target DNA molecules were commercially synthesized (Sangon Biotech, Shanghai, China), and mixed with the other PCR reactants to form a 10 μL amplification mixture as shown in Table 14 below. Particularly, an amplification mixture was made, containing 1.0×10⁻¹¹ M synthetic target microRNA fragments. The primer concentrations were at 3.0×10⁻⁶M, and polymerase concentration was 0.25 U/μL. A negative control group (NTC) of amplification mixture having the same contents but replacing the target microRNA fragment with water was included.

TABLE 14
Amplification mixture contents for microRNA amplification

| Content | Final Concentration |
|---|---|
| Primer 1 | 3.0 × 10⁻⁶M |
| Primer 2 | 3.0 × 10⁻⁶M |
| Target microRNA | 1.0 × 10⁻¹¹M |
| dNTPs | 8 mM |
| Isothermal | 1× |
| reaction buffer | |
| 20× Evagreen | 1.25 μL |
| Klenow Fragment exo- | 0.25 U/μL |
| Uracil-DNA Glycosylase | 0.05 U/μL |
| 100% polyethylene glycol 200 | 1.25 μL |
| Water | Make up to 20 μL total volume |

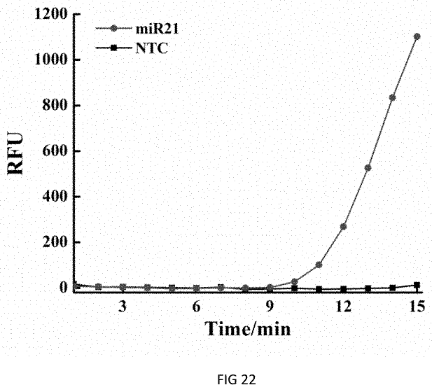

To carry out the accelerated SEA reaction, the amplification mixtures were subjected to swift thermal cycles between 60° C. and 34° C., using the CFX Connect™ RealTime PCR System (Bio-Rad, CA). Particularly, Particularly, each thermal cycle was constituted of incubating the amplification mixture at 60° C. for 1 second (s), before immediately reducing the temperature to 34° C., and incubating the amplification mixture at 34° C. for another 1 s, before immediately increasing the temperature back to 60° C. To monitor the amplification in real time, fluorescent signal emitted from the amplification mixture was scanned every two thermal cycles, and plotted over time in FIG. 22.

As shown in the figure, the accelerated SEA method was able to efficiently detect synthetic microRNA fragments within less than 10 minutes at the provided target concentrations, which enables the use of the present methods and kits for detection of microRNA from a sample.

### 6. SEQUENCE LISTING

This application is being filed with a computer readable form (CRF) copy of a Sequence Listing named 14624-002-228 ST25.TXT, created on Jan. 17, 2021, and being 14,125 bytes in size; which is incorporated herein by reference in its entirety.

1-118. (canceled)

119. A method for amplifying a target nucleic acid molecule in a sample, the method comprising
contacting a polymerase and a pair of oligonucleotide primers with the sample, thereby forming an amplification mixture; wherein the primers are configured to specifically hybridize to the target nucleic acid molecule;
subjecting the amplification mixture to a number of thermal cycles between a first temperature and a second temperature, thereby amplifying a sequence of the target nucleic acid molecule through polymerase chain reaction (PCR);
wherein the difference between the first and second temperatures is less than about 30° C.

120. The method of claim 119, wherein the difference between the first and second temperature is less than about 25° C. or less than about 20° C.,
preferably, wherein the difference between the first and second temperature is about 10-15° C.;
more preferably, the first and second temperatures is about 10° C., about 11° C., about 12° C., about 13° C., about 14° C., or about 15° C.

121. The method of claim 119, wherein the polymerase has an optimal temperature for catalyzing primer extension during the PCR;
Preferably, the optimal temperature is in the range of ±5° C. of the first temperature;
More preferably, the optimal temperature is in the range of ±6° C. of the second temperature;
Further more preferably, the optimal temperature is between the first and second temperatures.

122. The method of claim 119, wherein the sequence of the target nucleic acid molecule has a first melting temperature, and wherein the first temperature is in the range of ±5° C. of the first melting temperature.

123. The method of claim 119, wherein the pair of oligonucleotide primers have an average melting temperature, and wherein the second temperature is in the range of ±5° C. of the average melting temperature;
Preferably, the average melting temperature is within ±5° C. of the optimal temperature of the polymerase.

124. The method of claim 123, wherein one of the pair of oligonucleotide primers has a second melting temperature and the other one of the pair of oligonucleotide primers has a third melting temperature, and wherein difference between the second and third melting temperatures is less than about 3° C.

125. The method of claim 124, wherein the first melting temperature is determined using a computer algorithm based on the sequence of the target nucleic acid molecule, and wherein the second or third melting temperature is determined using a computer algorithm based on the sequence of the oligonucleotide primer;
Preferably, the computer algorithm is selected from NUPACK, DNAMelt, NOVPRO, BLAST, Primer Premier, AlignMiner, Oligo, PerlPrimer, Primer3Web and DNAstar.

126. The method of claim 124, wherein the method further comprises determining the first, second, third, and/or average melting temperature.

127. The method of claim 119, wherein the polymerase is a thermostable polymerase;
Preferably, wherein the polymerase has strand displacement activity;
More preferably, wherein the polymerase has reverse transcriptase activity;

128. The method of claim 127, wherein the polymerase is Bst DNA polymerase, or an isomerase thereof, or a functional derivative having at least 80% sequence identity thereof, preferably the polymerase is Bst DNA polymerase Large Fragment, or isomerase thereof, or a functional mutant having at least 80% sequence identity thereof; or preferably the polymerase is full length Bst DNA Polymerase, Bst DNA Polymerase Large Fragment, Bst 2.0 DNA polymerase, Bst 2.0 WarmStart DNA Polymerase, or Bst 3.0 DNA polymerase; more preferably, the first temperature is in the range of about 68-78° C., and the second temperature is in the range of about 55-69° C.;
or, wherein the polymerase is DNA Polymerase I, or an isomerase thereof, or a functional mutant having at least 80% sequence identity thereof; preferably, the polymerase is DNA Polymerase I Large (Klenow) Fragment, or an isomerase thereof, or a functional mutant having at least 80% sequence identity thereof; or preferably, the polymerase is wild-type DNA Polymerase I, DNA Polymerase I Large (Klenow) Fragment, or Klenow exo⁻; more preferably, the first temperature is in the range of about 50-60° C., and the second temperature is in the range of about 30-40° C.;
or, wherein the polymerase is a Vent DNA polymerase, or an isomerase thereof, or a functional mutant having at least 80% sequence identity thereof; preferably, the polymerase is Vent DNA polymerase, Vent (exo⁻) DNA polymerase, Deep Vent DNA polymerase, or Deep Vent (exo⁻) DNA polymerase; more preferably, the first temperature is in the range of about 70-80° C., and the second temperature is in the range of about 55-70° C.;
or, wherein the polymerase is a phi29 DNA polymerase, or an isomerase thereof, or a functional mutant having at least 80% sequence identity thereof; preferably, the first temperature is selected from the range of about 40-55° C., and the second temperature is selected from the range of about 20-37° C.;
or, wherein the polymerase is a Taq DNA polymerase, or an isomerase thereof, or a functional mutant having at least 80% sequence identity thereof; preferably, the polymerase is Taq DNA polymerase, Hot Start Taq DNA Polymerase, EpiMark Hot Start Taq DNA Polymerase, OneTaq DNA Polymerase, One Taq Hot Start DNA Polymerase, LongAmp Taq DNA Polymerase, or Long Taq DNA Polymerase; more preferably the first temperature is in the range of about 70-88° C., and the second temperature is in the range of about 58-70° C.

129. The method of claim 119, wherein the ratio of the length of the amplified sequence and the length of at least one of the primers is in the range of about 30-60%;
preferably, wherein the amplified sequence is about 20-50 base pair (bp) long;
more preferably, wherein the primer is about 10 to about 25 nucleotides (nt) long.

130. The method of claim 119, wherein at least one of the primers has a G/C content in the range of about 40% to about 60%, and wherein the difference between the G/C content of the primers are less than 20%;
or, wherein at least one of the primers has an elongation terminus where the polymerase adds nucleotides during the PCR, and wherein the primer has G or C at the elongation terminus;
or, wherein at least one of the primers has an elongation terminus where the polymerase adds nucleotides during the PCR, and wherein the primer has a G/C content of at least 40% in a continuous 5-nucletoide region including the elongation terminus.

131. The method of claim 119, wherein each thermal cycle comprises incubating the amplification mixture at the first temperature for less than 2 s and incubating the amplification mixture at the second temperature for less than 2 s;
preferably, wherein each thermal cycle further comprises a total ramp time of less than 10 s;
more preferably, wherein each thermal cycle comprises incubating the amplification mixture at the first temperature for about 1 s and incubating the amplification mixture at the second temperature for about 1 s, and wherein the ramp time is less than 2 s;
further more preferably, wherein the method completes at least 35 thermal cycles in less than 10 minutes, or completes at least 40 thermal cycles in less than 8 minutes.

132. The method of claim 119, wherein the amplification mixture further comprises dUTPs.
and/or, wherein the amplification mixture does not contain dTTPs;
and/or, wherein the amplification mixture further comprises uracil-DNA glycosylase (UDG);
and/or, wherein the amplification mixture further comprises a single strand binding protein (SSB);
and/or, wherein the amplification mixture further comprises polyethylene glycol;
and/or, wherein the amplification mixture comprise the target nucleic acid of no more than 1.0×10⁻¹²M;
and/or, wherein the amplification mixture comprises less than 10 copies of the target nucleic acid;
and/or, wherein the amplification mixture comprises the polymerase at a concentration of no less than 0.1 U/μL;
and/or, wherein the amplification mixture comprises at least one of the primers at a concentration of no less than 1.0×10⁻⁶ M;
and/or, wherein the amplification mixture comprises polyethylene glycol of at least 0.5% by volume;
and/or, wherein the amplification mixture comprises the SSB at a concentration of at least 1 μg/mL;
and/or, wherein the amplification mixture has a volume of about 1-30 μL;
and/or, wherein the subjecting step is performed by loading the amplification mixture onto a microfluidic device capable of cooling and heating the amplification mixture at a speed of at least 10° C./s;
and/or, wherein the target nucleic acid is a double-stranded nucleic acid molecule, or single-stranded nucleic acid molecule;
and/or, wherein the target nucleic acid is DNA or RNA;
and/or, wherein the target nucleic acid is microRNA.

133. A method for using the method of claim 119, is (I), (II) or (III):
(I) A method for detecting a target nucleic acid molecule in a sample comprising
contacting a polymerase and a pair of oligonucleotide primers with the sample, thereby forming an amplification mixture; wherein the primers are configured to specifically hybridize to the target nucleic acid molecule;
subjecting the amplification mixture to a number of thermal cycles between a first temperature and a second temperature, thereby amplifying a sequence of the target nucleic acid molecule through polymerase chain reaction (PCR); wherein the difference between the first and second temperatures is less than about 30° C.; and
detecting the amplified sequence in the amplification mixture.
(II) A method for diagnosing an infection by a pathogen in a subject comprising
providing a nucleic acid containing sample collected from the subject;
contacting a polymerase and a pair of oligonucleotide primers with the sample, thereby forming an amplification mixture; wherein the primers are configured to amplify a pathogenic sequence indicative of the pathogen infection;
subjecting the amplification mixture to a number of thermal cycles between a first temperature and a second temperature, thereby amplifying the pathogenic sequence through polymerase chain reaction (PCR); wherein the difference between the first and second temperatures is less than about 30° C.; and
detecting the presence or absence of the amplified sequence in the amplification mixture.
(III) A method for detecting a genetic alteration in a subject, comprising
providing a nucleic acid containing sample collected from the subject;
contacting a polymerase and a pair of oligonucleotide primers with the sample, thereby forming an amplification mixture; wherein the primers are configured to amplify a target sequence from the subject's genome suspected of containing the genetic alteration;
subjecting the amplification mixture to a number of thermal cycles between a first temperature and a second temperature, thereby amplifying the target sequence through polymerase chain reaction (PCR); wherein the difference between the first and second temperatures is less than about 30° C.; and
sequencing the amplified sequence to determine the presence of absence of the genetic alteration.

134. The method of claim 133, wherein in the method (I), the detecting is performed every 1, 2, 5 or 10 thermal cycles; preferably, the detecting is performed by detecting a fluorescent signal reflective of the amount of the amplified sequence in the amplification mixture;
or, wherein in the method (II), the sample contains extracted genomic nucleic acid of the subject, or cell-free nucleic acid from the subject; preferably, the sample is a bodily fluid sample; more preferably, the pathogen is virus, bacteria, fungi or parasite;
or, wherein in the method (III), the genetic alteration is a gene mutation selected from nucleotide substitute, deletion, insertion or copy number variation; preferably, the genetic alteration is single nucleotide polymorphism; more preferably, the method further comprising diagnosing or prognosing a genetic condition associated with the genetic alteration.

135. A kit for amplifying a target nucleic acid molecule comprising a plurality of components comprising a thermostable polymerase and a pair or oligonucleotide primers,
wherein the pair of primers are configured to amplify, through polymerase chain reaction (PCR), an amplification region of about 20-50 base pairs (bp) in the target nucleic acid; and
wherein the thermostable polymerase comprises strand displacement activity.

136. The kit of claim 135, wherein at least one of the primers have a melting temperature within ±5° C. of the optimal temperature of the thermostable polymerase;
and/or, wherein at least one of the primers has a G/C content in the range of about 40%-60%;
and/or, wherein each primer comprises an elongation terminus where the polymerase adds nucleotides during the PCR, and wherein at least one of the primers has a G/C content of at least 40% in a continuous 5-nucleotide region including the elongation terminus;
and/or, wherein each primer comprises an elongation terminus where the polymerase adds nucleotides during the PCR, and wherein at least one of the primers has G or C at the elongation terminus;
and/or, wherein at least one of the primers is about 10-25 nucleotides long.

137. The kit of claim 135, wherein the polymerase is Bst DNA polymerase, or an isomerase thereof, or a functional mutant having at least 80% sequence identity thereof;
and/or, wherein the polymerase is Bst DNA polymerase Large Fragment, or an isomerase thereof, or a functional mutant having at least 80% sequence identity thereof;
and/or, wherein the polymerase is full length Bst DNA Polymerase, Bst DNA Polymerase Large Fragment, Bst 2.0 DNA polymerase, Bst 2.0 WarmStart DNA Polymerase, or Bst 3.0 DNA polymerase;
and/or, wherein the polymerase is DNA Polymerase I, or an isomerase thereof, or a functional mutant having at least 80% sequence identity thereof;
and/or, wherein the polymerase is DNA Polymerase I Large (Klenow) Fragment, or an isomerase thereof, or a functional mutant having at least 80% sequence identity thereof;
and/or, wherein the polymerase is wild-type DNA Polymerase I, DNA Polymerase I Large (Klenow) Fragment, or Klenow exo⁻;
and/or, wherein the polymerase is a Vent DNA polymerase, or an isomerase thereof, or a functional mutant having at least 80% sequence identity thereof;
and/or, wherein the polymerase is Vent DNA polymerase, Vent (exo⁻) DNA polymerase, Deep Vent DNA polymerase, or Deep Vent (exo⁻) DNA polymerase;
and/or, wherein the polymerase is a phi29 DNA polymerase, or an isomerase thereof, or a functional mutant having at least 80% sequence identity thereof;
and/or, wherein the polymerase is a Taq DNA polymerase, or an isomerase thereof, or a functional mutant having at least 80% sequence identity thereof;
and/or, wherein the polymerase is Taq DNA polymerase, Hot Start Taq DNA Polymerase, EpiMark Hot Start Taq DNA Polymerase, OneTaq DNA Polymerase, OneTaq Hot Start DNA Polymerase, LongAmp Taq DNA Polymerase, or Long Taq DNA Polymerase.

138. The kit of claim 135, wherein further comprising dUTPs;
and/or, wherein the kit does not contain dTTPs;
and/or, further comprising uracil-DNA glycosylase (UDG);
and/or, further comprising a buffer solution suitable for the polymerase;
and/or, wherein the kit further comprises a single strand binding protein (SSB), preferably a thermal stable SSB;
and/or, wherein the SSB protein is originated from bacteria or phage;
and/or, wherein the SSB protein is selected from T4 phage 32 SSB, T7 phage 2.5 SSB, phi phage 29 SSB, E. coli SSB, or functional derivative thereof;
and/or wherein, further comprising polyethylene glycol.
and/or, wherein the plurality of components are
(a) contained in one container, and the kit further comprises an instruction of adding a suitable amount of sample to form an amplification mixture; or
(b) contained in at least two separate containers, and wherein the kit further comprises an instruction of mixing the components in the separate containers and a suitable amount of sample to form an amplification mixture;
and/or, wherein the amplification mixture comprises the polymerase at a concentration of no less than 0.1 U/μL;
and/or, wherein the amplification mixture comprises at least one of the primers at a concentration of no less than 1.0×10⁻⁶ M;
and/or, wherein the amplification mixture comprises polyethylene glycol of about 0.5%-1.0×10% by volume;
and/or, wherein the amplification mixture comprises the SSB at a concentration of about 1-50 μg/mL;
and/or, wherein the amplification mixture has a volume of about 1-30 μL;
and/or, wherein the kit further comprises an instruction for performing the PCR using a thermal cycling protocol comprising a number of thermal cycles, wherein each thermal cycle comprises incubation at a first temperature for no more than 2 s, and incubation at a second temperature for no more than 2 s, and wherein the difference between the first and second temperatures is less than 30° C.;
and/or, wherein the polymerase is full length Bst DNA Polymerase, Bst DNA Polymerase Large Fragment, Bst 2.0 DNA polymerase, Bst 2.0 WarmStart DNA Polymerase, or Bst 3.0 DNA polymerase, and wherein the first temperature is in the range of about 68-78° C., and the second temperature is in the range of about 55-69° C.;
and/or, wherein the polymerase is wild-type DNA Polymerase I, DNA Polymerase I Large (Klenow) Fragment, or Klenow exo⁻, and wherein the first temperature is in the range of about 40-55° C., and the second temperature is in the range of about 20-37° C.;
and/or, wherein the polymerase is Vent DNA polymerase, Vent (exo⁻) DNA polymerase, Deep Vent DNA polymerase, or Deep Vent (exo⁻) DNA polymerase, and wherein the first temperature is in the range of about 70-80° C., and the second temperature is in the range of about 55-70° C.
and/or, wherein the polymerase is phi29 DNA polymerase, and wherein the first temperature is selected from the range of about 40-55° C., and the second temperature is selected from the range of about 20-37° C.;
and/or, wherein the polymerase is Taq DNA polymerase, Hot Start Taq DNA Polymerase, EpiMark Hot Start Taq DNA Polymerase, One Taq DNA Polymerase, One Taq Hot Start DNA Polymerase, LongAmp Taq DNA Polymerase, or long Taq DNA Polymerase, and wherein the first temperature is selected from the range of about 70-88° C. and the second temperature is selected from the range of about 58-70° C.;
and/or, wherein each thermal cycle further comprises a total ramp time of less than 10 s;
and/or, wherein the number of thermal cycles is less than 40 cycles and the thermal cycling protocol further comprises a total reaction time of less than 10 minutes;
and/or, wherein each thermal cycle comprises incubation at the first temperature selected from the range of about 72-76° C. for about 1 s, and incubation at the second temperature selected from the range of about 61-65° C. for about 1 s, and the total ramp time of less than 2 s, and wherein the total reaction time is less than 8 minutes;
and/or, wherein the amplification region has a first melting temperature, and wherein the first temperature is in the range of ±5° C. of the first melting temperature
and/or, wherein the pair of primers have an average melting temperature, and wherein the second temperature is in the range of ±5° C. of the average melting temperature;
and/or, wherein one of the pair of primers has a second melting temperature and the other one of the pair of primers have a third melting temperature, and wherein difference between the second and third melting temperatures is less than about 3° C.


SEQUENCE LISTING (69 sequences)

SEQ ID NO: 1 - DNA, 50 bp, Artificial Sequence
  Synthesized
gggtcattggaaactggaagactggagtgcagaagaggagagtggaattc

SEQ ID NO: 2 - DNA, 22 bp, Artificial Sequence
  Synthesized
gtcattggaaactggaagactg

SEQ ID NO: 3 - DNA, 19 bp, Artificial Sequence
  Synthesized
ccactctcctcttctgcac

SEQ ID NO: 4 - DNA, 50 bp, Artificial Sequence
  Synthesized
ggttcaaaagtgaaagacggtcttgctgtcacttatagatggatccgcgc

SEQ ID NO: 5 - DNA, 25 bp, Artificial Sequence
  Synthesized
ggttcaaaagtgaaagacggtcttg

SEQ ID NO: 6 - DNA, 23 bp, Artificial Sequence
  Synthesized
gcgcggatccatctataagtgac

SEQ ID NO: 7 - DNA, 50 bp, Artificial Sequence
  Synthesized
gggtcauuggaaacuggaagacuggagugcagaagaggagaguggaauuc

SEQ ID NO: 8 - DNA, 21 bp, Artificial Sequence
  Synthesized
tcgcggtaatacataggtcgc

SEQ ID NO: 9 - DNA, 22 bp, Artificial Sequence
  Synthesized
gcccaataaatccggataacgc

SEQ ID NO: 10 - DNA, 19 bp, Artificial Sequence
  Synthesized
gtctggtgttaaaggcagc

SEQ ID NO: 11 - DNA, 23 bp, Artificial Sequence
  Synthesized
tccaatgcatacaactgttaagc

SEQ ID NO: 12 - DNA, 21 bp, Artificial Sequence
  Synthesized
gcaagggttcgttatttgatg

SEQ ID NO: 13 - DNA, 18 bp, Artificial Sequence
  Synthesized
ctagctgatatggcgcac

SEQ ID NO: 14 - DNA, 22 bp, Artificial Sequence
  Synthesized
gctatgctgagaagtagaatag

SEQ ID NO: 15 - DNA, 18 bp, Artificial Sequence
  Synthesized
gtgtctcagtcccattgt

SEQ ID NO: 16 - DNA, 20 bp, Artificial Sequence
  Synthesized
aatgactttagcaggtaatg

SEQ ID NO: 17 - DNA, 18 bp, Artificial Sequence
  Synthesized
tggtacagtcaaactcta

SEQ ID NO: 18 - DNA, 23 bp, Artificial Sequence
  Synthesized
gtctaggcggattgagagattgg

SEQ ID NO: 19 - DNA, 21 bp, Artificial Sequence
  Synthesized
gtgtctcagtcccagtgttgg

SEQ ID NO: 20 - DNA, 26 bp, Artificial Sequence
  Synthesized
tagtaatgcatagataatttgtcctt

SEQ ID NO: 21 - DNA, 22 bp, Artificial Sequence
  Synthesized
gtttccaaccgttattcccaag

SEQ ID NO: 22 - DNA, 23 bp, Artificial Sequence
  Synthesized
atcttaggacctttcggttaagg

SEQ ID NO: 23 - DNA, 23 bp, Artificial Sequence
  Synthesized
actagctgatatcacatagactc

SEQ ID NO: 24 - DNA, 19 bp, Artificial Sequence
  Synthesized
ccaccaaacacatgcatac

SEQ ID NO: 25 - DNA, 21 bp, Artificial Sequence
  Synthesized
catgggcttgggtttactatg

SEQ ID NO: 26 - DNA, 20 bp, Artificial Sequence
  Synthesized
agcactatccatcaccattg

SEQ ID NO: 27 - DNA, 20 bp, Artificial Sequence
  Synthesized
agggtgaagtatacgcctag

SEQ ID NO: 28 - DNA, 19 bp, Artificial Sequence
  Synthesized
gacccaccaaacacatgca

SEQ ID NO: 29 - DNA, 21 bp, Artificial Sequence
  Synthesized
gggcttgggtttactatgtgg

SEQ ID NO: 30 - DNA, 21 bp, Artificial Sequence
  Synthesized
gcaagggttcgttatttgatg

SEQ ID NO: 31 - DNA, 18 bp, Artificial Sequence
  Synthesized
ctagctgatatggcgcac

SEQ ID NO: 32 - DNA, 18 bp, Artificial Sequence
  Synthesized
gacatccttggcaaagtt

SEQ ID NO: 33 - DNA, 22 bp, Artificial Sequence
  Synthesized
cggttaacctccattatgtttc

SEQ ID NO: 34 - DNA, 20 bp, Artificial Sequence
  Synthesized
cgcataagaactttggttcg

SEQ ID NO: 35 - DNA, 24 bp, Artificial Sequence
  Synthesized
gcaggtcctttcaactttgattca

SEQ ID NO: 36 - DNA, 23 bp, Artificial Sequence
  Synthesized
gtctaggcggattgagagattgg

SEQ ID NO: 37 - DNA, 21 bp, Artificial Sequence
  Synthesized
gtgtctcagtcccagtgttgg

SEQ ID NO: 38 - DNA, 19 bp, Artificial Sequence
  Synthesized
gtaaaggcctaccaaggct

SEQ ID NO: 39 - DNA, 19 bp, Artificial Sequence
  Synthesized
ctctcaatccgcctagacg

SEQ ID NO: 40 - DNA, 22 bp, Artificial Sequence
  Synthesized
cgttaaagaaggggatcttagg

SEQ ID NO: 41 - DNA, 22 bp, Artificial Sequence
  Synthesized
atagactctcccttaaccgaaa

SEQ ID NO: 42 - DNA, 23 bp, Artificial Sequence
  Synthesized
gtctaggcggattgagagattgg

SEQ ID NO: 43 - DNA, 21 bp, Artificial Sequence
  Synthesized
gtgtctcagtcccagtgttgg

SEQ ID NO: 44 - DNA, 20 bp, Artificial Sequence
  Synthesized
ctttcggttaagggagagtc

SEQ ID NO: 45 - DNA, 21 bp, Artificial Sequence
  Synthesized
ccaccaactagctgatatcac

SEQ ID NO: 46 - DNA, 26 bp, Artificial Sequence
  Synthesized
tagtaatgcatagataatttgtcctt

SEQ ID NO: 47 - DNA, 22 bp, Artificial Sequence
  Synthesized
gtttccaaccgttattcccaag

SEQ ID NO: 48 - DNA, 18 bp, Artificial Sequence
  Synthesized
gactgccggtgacaaacc

SEQ ID NO: 49 - DNA, 18 bp, Artificial Sequence
  Synthesized
cgtcatccccaccttcct

SEQ ID NO: 50 - DNA, 19 bp, Artificial Sequence
  Synthesized
gttgcgacagctctaggac

SEQ ID NO: 51 - DNA, 19 bp, Artificial Sequence
  Synthesized
ctgcaccaccgataattgc

SEQ ID NO: 52 - DNA, 21 bp, Artificial Sequence
  Synthesized
cgctagtaatcgcggatcagc

SEQ ID NO: 53 - DNA, 18 bp, Artificial Sequence
  Synthesized
cgggaacgtattcaccgc

SEQ ID NO: 54 - DNA, 25 bp, Artificial Sequence
  Synthesized
ggttcaaaagtgaaagacggtcttg

SEQ ID NO: 55 - DNA, 23 bp, Artificial Sequence
  Synthesized
gcgcggatccatctataagtgac

SEQ ID NO: 56 - DNA, 19 bp, Artificial Sequence
  Synthesized
cgcatggttcaaaagtgaa

SEQ ID NO: 57 - DNA, 17 bp, Artificial Sequence
  Synthesized
agtgacagcaagaccgt

SEQ ID NO: 58 - DNA, 51 bp, Artificial Sequence
  Synthesized
agatgttgaaggattcaaccaaatctccagagtttgttaaaaccgttccaa

SEQ ID NO: 59 - DNA, 23 bp, Artificial Sequence
  Synthesized
atgttgaaggattcaaccaaatc

SEQ ID NO: 60 - DNA, 22 bp, Artificial Sequence
  Synthesized
ggaacggttttaacaaactctg

SEQ ID NO: 61 - DNA, 66 bp, Artificial Sequence
  Synthesized
caaatgcttctaggcggactatgacttagttgcgttacaccctttcttgacaaaacctaacttgcg

SEQ ID NO: 62 - DNA, 20 bp, Artificial Sequence
  Synthesized
cctgtgttatcttggaggtc

SEQ ID NO: 63 - DNA, 18 bp, Artificial Sequence
  Synthesized
ccctgaagggctctctgg

SEQ ID NO: 64 - DNA, 20 bp, Artificial Sequence
  Synthesized
accaaaagagctagaaccac

SEQ ID NO: 65 - DNA, 19 bp, Artificial Sequence
  Synthesized
atgttgaaggattcaacca

SEQ ID NO: 66 - DNA, 19 bp, Artificial Sequence
  Synthesized
ggaacggttttaacaaact

SEQ ID NO: 67 - RNA, 20 nt, Artificial Sequence
  Synthesized
gcuuaucagacugauguuga

SEQ ID NO: 68 - DNA, 10 bp, Artificial Sequence
  Synthesized
gcttatcaga

SEQ ID NO: 69 - DNA, 10 bp, Artificial Sequence
  Synthesized
tcaacatcag